(19) United States
(12) Patent Application Publication
(10) Pub. No.: US 2020/0108066 A1
(43) Pub. Date: Apr. 9, 2020

(54) METHODS FOR MODULATING REGULATORY T CELLS AND IMMUNE RESPONSES USING CDK4/6 INHIBITORS

(71) Applicants: Shorn Goel, Brookline, MA (US); Jean ZHAO, Brookline, MA (US); Flye-Jung KIM, Brookline, MA (US); Sandra S. MCALLISTER, Needham, MA (US); Molly DECRISTO, Brookline, MA (US); Dana-Farber Cancer Institute, Inc., Boston, MA (US); The Brigham and Women's Hospital, Inc., Boston, MA (US)

(72) Inventors: Shom Goel, Brookline, MA (US); Jean Zhao, Brookline, MA (US); Flye-Jung Kim, Brookline, MA (US); Sandra S. McAllister, Needham, MA (US); Molly DeCristo, Brookline, MA (US)

(21) Appl. No.: 16/497,297

(22) PCT Filed: Mar. 28, 2018
(86) PCT No.: PCT/US18/24818

§ 371 Date: Sep. 24, 2019

Related U.S. Application Data
(60) Provisional application No. 62/478,909, filed on Mar. 30, 2017.

(51) Int. Cl. A61K 31/506; A61P 35/00; A61K 45/06

(52) U.S. Cl. CPC A61K 31/506; A61K 45/06; A61P 35/00

(57) ABSTRACT

The present invention is based, in part, on methods for modulating regulatory T cells and immune responses using CDK4/6 inhibitors.

### CROSS-REFERENCE TO RELATED APPLICATIONS

This application claims the benefit of U.S. Provisional Application No. 62/478,909, filed on 30 Mar. 2017; the entire contents of said application are incorporated herein in their entirety by this reference.

### STATEMENT OF RIGHTS

This invention was made with government support under Grants P50 CA168504, CA187918-02; CA210057-01; CA172461-04, and R01 CA166284 awarded by The National Institutes of Health. The government has certain rights in the invention.

### BACKGROUND OF THE INVENTION

The cyclin-dependent kinases CDK4 and CDK6 are fundamental regulators of cell cycle progressionl. Upon binding to D-type cyclins, CDKs 4 and 6 phosphorylate the retinoblastoma tumor suppressor (Rb). Consequently, E2F transcription factors are released from Rb-mediated inactivation, thus enabling expression of genes promoting progression through G1 to the S phase of the cell cycle (Sherr & Roberts (2004) Genes Dev 18:2699-2711; Narasimha et al. (2014) Elife 3:e02872). Preclinical studies demonstrate that CDKs 4 and 6 are required for initiating and maintaining growth of solid tumors including breast cancers (Yu et al. (2006) Cancer Cell 9:23-32; Choi et al. (2012) Cancer Cell 22:438-451; Sherr et al. (2016) Cancer Discov 6:353-367). Recently, selective pharmacologic inhibitors of CDK4/6 have been developed (Finn et al. (2015) Lancet Oncol 16:25-35; Patnaik et al. (2016) Cancer Discov 6:740-753; Hortobagyi et al. (2016) N Engl J Med 375:1738-1748). As expected, these compounds induce G1 cell cycle arrest in many Rb-expressing breast cancer cell lines (Finn et al. (2009) Breast Cancer Res 11:R77; Vora et al. (2014) Cancer Cell 26:136-149; Goel et al. (2016) Cancer Cell 29:255-269). One such inhibitor, palbociclib, has received US FDA approval as treatment for estrogen receptor-positive breast cancer and two others (abemaciclib and ribociclib) have shown promising results in this same patient population (Patnaik et al. (2016), supra; Hortobagyi et al. (2016), supra).

In clinical trials, CDK4/6 inhibitor monotherapy has resulted in objective tumor responses (reductions in tumor size of over 30 percent) in several patients with metastatic breast cancer. Response rates were highest with abemaciclib (dosed continuously), but tumor regression has also resulted from therapy with palbociclib (dosed intermittently) (Patnaik et al. (2016), supra; DeMichele et al. (2015) Clin Cancer Res 21:995-1001). The reason for tumor regression after CDK4/6 inhibition is unclear. Recent pre-clinical studies have demonstrated that the effects of CDK4/6 inhibitors may extend beyond G1 growth arrest. As an example, CDK4/6 inhibition resulted in a senescence-like state in tumor cells (Choi et al. (2012), supra; Goel et al. (2016), supra; Anders et al. (2011) Cancer Cell 20:620-634). However, solid tumor cell apoptosis has not been convincingly demonstrated with these agents (Choi et al. (2012), supra; Vora et al. (2014), supra; Goel et al. (2016), supra; Puyol et al. (2010) Cancer Cell 18:63-73; Witkiewicz et al. (2014) Genes Cancer 5:261-272) and cellular cytostasis alone would not explain tumor shrinkage. Moreover, very little is known about the effects of CDK4/6 inhibitors on cells within the tumor microenvironment. Concerns were raised that CDK4/6 inhibition might render immune checkpoint therapy less effective due to T cell cycle inhibition (Sherr (2016) N Engl J Med 375:1920-1923). Accordingly, a great need exists in the art to identify and better understand immunomodulatory agents and methods of use thereof.

### SUMMARY OF THE INVENTION

The present invention is based, at least in part, on the discovery that CDK4/6 inhibitors selectively reduce the number of circulating regulatory T cells (Tregs) in a subject. For example, the Tregs in the spleen and/or lymph nodes, but not in the thymus, in a subject are significantly reduced by CDK4/6 inhibitors. On the contrary, other types of T cells remain unchanged. Such reduction of circulating Tregs contributes towards T cell-mediated cytotoxicity. The reason behind the specific reduction in Treg numbers is, at least in part, related to suppression of DNA methyltransferase 1 levels in Tregs, which in turn further enhances their cell cycle arrest. In the context of cancer, CDK4/6 inhibitors also increase tumor cell antigen presentation by increasing type 3 interferon production and expression of interferon-sensitive genes (ISGs). Thus, the results provided herein demonstrate that CDK4/6 inhibitors can be used to treat cancers by promoting anti-tumor immunity (e.g., through enhanced tumor cell antigen presentation and anti-tumor T cell responses). CDK4/6 inhibitors can be used alone or in combination with immune checkpoint therapy to treat cancers. Accordingly, the present invention relates, in part, to methods of upregulating an immune response such as is beneficial in treating cancers (e.g., through selectively reducing the number of circulating Tregs) in a subject with at least one CDK4/6 inhibiting agent, alone or in combination with an immunotherapy, such as an immune checkpoint inhibitor therapy.

In one aspect, a method of selectively reducing the number of circulating regulatory T cells (Tregs) in a subject, comprising administering to the subject a therapeutically effective amount of at least one agent that selectively inhibits or blocks the expression or activity of CDK4 and/or CDK6 such that the number of Tregs in the subject is selectively reduced, is provided.

Numerous embodiments are further provided that can be applied to any aspect of the present invention and/or combined with any other embodiment described herein. For example, in one embodiment, the Tregs comprise CD4+CD25+, CD4+FOXP3+, and/or CD4+CD25+FOXP3+ Tregs. In another embodiment, the at least one agent significantly reduces the number of the Tregs in the spleen and/or lymph nodes of the subject. In still another embodiment, the at least one agent does not significantly reduce the number of the Tregs in the thymus of the subject. In yet another embodiment, the at least one agent does not significantly affect differentiation of naïve CD4+ T cells into Tregs in the subject. In another embodiment, the at least one agent does not significantly affect Treg apoptosis in the subject. In still another embodiment, the at least one agent does not significantly change the cell number of at least one cell type selected from the group consisting of B lymphocytes, natural killer cells, neutrophils, and monocytes. In yet another embodiment, the at least one agent reduces the ratio of Tregs to CD3+ T cells and/or the ratio of Tregs to CD8+ T cells in the subject. In another embodiment, the at least one agent does not significantly modulate the number of CD8+ T cells and/or CD4+CD25− T cells. In still another embodiment, the at least one agent reduces the expression of at least one marker selected from the group consisting of PD-1, TIM-3, CTLA-4, and LAG3 on the surface of CD4+ and/or CD8+ T cells. In yet another embodiment, the at least one agent increases antigen presentation in the subject. In another embodiment, the at least one agent increases MHC class I expression in the subject. In still another embodiment, the at least one agent increases T cell-mediated cytotoxicity in the subject. In yet another embodiment, the at least one agent increases interferon (e.g., type III interferon) production, signaling, and/or secretion in the subject. In another embodiment, the at least one agent increases expression of at least one gene selected from the group consisting of STAT1, STAT2, IRF2, IRF6, IRF7, IRF9, NLRC5, OAS1, OAS2, IFIT1, IFIT2, IFIT6, BST2, SP100, RSAD2, CXCL9, CXCL10, CXCL11, Icam1, Vcam1, IL-29, IL-28a, IL-28b, ERV3-1, ERVK13-1, RIG-1, LGP2, and MDA5 in the subject, or any ISG described herein, such as those listed in Table 2. In still another embodiment, the at least one agent inhibits at least one DNA methyltransferase (DNMT, such as DNMT1) in the subject. In yet another embodiment, the at least one agent does not significantly enhance senescence associated secretory phenotype (SASP) in the subject. In another embodiment, the at least one agent is selected from the group consisting of: a small molecule CDK4 antagonist, a blocking intrabody or antibody that binds CDK4, a non-activating form of CDK4, a soluble form of an CDK4 natural binding partner, a CDK4 fusion protein, a nucleic acid molecule that blocks CDK4 transcription or translation, a small molecule CDK6 antagonist, a blocking intrabody or antibody that recognizes CDK6, a non-activating form of CDK6, a soluble form of a CDK6 natural binding partner, a CDK6 fusion protein, and a nucleic acid molecule that blocks CDK6 transcription or translation. In still another embodiment, said at least one agent comprises a small molecule (e.g., abemaciclib, palbociclib, and ribociclib) that inhibits or blocks CDK4 and/or CDK6 expression or activity. In yet another embodiment, said at least one agent comprises an RNA interfering agent (e.g., a small interfering RNA (siRNA), small hairpin RNA (shRNA), microRNA (miRNA), or a piwiRNA (piRNA)) which inhibits or blocks CDK4 and/or CDK6 expression or activity. In another embodiment, said at least one agent comprises an antisense oligonucleotide complementary to CDK4 and/or CDK6. In still another embodiment, said at least one agent comprises a peptide or peptidomimetic that inhibits or blocks CDK4 and/or CDK6 expression or activity. In yet another embodiment, said at least one agent comprises an aptamer that inhibits or blocks CDK4 and/or CDK6 expression or activity. In another embodiment, said at least one agent is an intrabody or antibody, or an antigen binding fragment thereof, which specifically binds to CDK4 and/or CDK6. In still another embodiment, said intrabody or antibody, or antigen binding fragment thereof, is murine, chimeric, humanized, or human. In yet another embodiment, said intrabody or antibody, or antigen binding fragment thereof, is detectably labeled, comprises an effector domain, comprises an Fc domain, and/or is selected from the group consisting of Fv, F(ab′)2, Fab′, dsFv, scFv, sc(Fv)2, and diabody fragments. In another embodiment, said at least one agent is administered in a pharmaceutically acceptable formulation.

In still another embodiment, the subject has a condition that would benefit from upregulation of an immune response. In still another embodiment, the subject has a condition selected from the group consisting of a cancer, a viral infection, a bacterial infection, a protozoal infection, a helminth infection, asthma associated with impaired airway tolerance, and an immunosuppressive disease. In yet another embodiment, the condition is a cancer (e.g., breast cancer, colorectal cancer, etc.). In another embodiment, at least some of the subject's immune cells, Tregs, or cancer cells express Rb and/or has functional Rb signaling. In still another embodiment, at least some of the subject's immune cells, Tregs, or cancer cells have defective Rb expression and/or defective Rb signaling. In yet another embodiment, at least some of the subject's Tregs or cancer cells harbor genomic mutations causing defective Rb expression and/or defective Rb signaling. In another embodiment, the condition is resistant to immune checkpoint blockade. In still another embodiment, the at least one agent increases the susceptibility to immune checkpoint blockade of the subject's cells, immune cells, Tregs, or cancer cells in the subject. In yet another embodiment, the at least one agent: a) increases the number of cancer infiltrating CD3+ T cells in the subject; b) increases antigen presentation by cancer cells in the subject; c) increases MHC class I expression by cancer cells in the subject; d) increases interferon production, signaling, and/or secretion by cancer cells in the subject; e) increases type III interferon production, signaling, and/or secretion by cancer cells in the subject; f) increases expression of at least one gene selected from the group consisting of STAT1, STAT2, IRF2, IRF6, IRF7, IRF9, NLRC5, OAS1, OAS2, IFIT1, IFIT2, IFIT6, BST2, SP100, RSAD2, CXCL9, CXCL10, CXCL11, Icam1, Vcam1, IL-29, IL-28a, IL-28b, ERV3-1, ERVK13-1, RIG-1, LGP2, and MDA5 by cancer cells in the subject; g) inhibits expression of at least one DNA methyltransferase (DNMT) by cancer cells in the subject; and/or h) inhibits expression of DNMT1 expression by cancer cells in the subject.

In one embodiment, the method further comprises administering one or more additional agents or therapies that upregulates an immune response. In another embodiment,

the one or more additional agents or therapies is selected from the group consisting of immunotherapy, a vaccine, chemotherapy, radiation, epigenetic modifiers, and targeted therapy. Such immunotherapy may, e.g., be selected from the group consisting of immune checkpoint inhibitor therapy, a sensitized antigen presenting cell, an oncolytic virus, an expression vector comprising an anticancer gene, and an inhibitor of a cancer antigen or a disease antigen. Such immune checkpoint inhibitor therapy may comprise, e.g., reducing or inhibiting the expression and/or function of an immune checkpoint molecule selected from the group consisting of CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR in the subject. In one embodiment, the immune checkpoint inhibitor therapy targets an immune checkpoint selected from the group consisting of PD-1, CTLA-4, PD-L1, PD-L2, and combinations thereof. In another embodiment, the at least one agent described herein is administered prior to administering the one or more additional agents or therapies that upregulates the immune response, optionally wherein the at least one agent is preadministered before subsequent administration of a combination of the at least one agent and the one or more additional agents or therapies that upregulates the immune response. In one embodiment, the subject described herein is a mammal. For example, the mammal may be an animal model of the condition, or a human.

In another aspect, a method of upregulating an immune response in a subject in need thereof, comprising administering to the subject a combination of i) a therapeutically effective amount of at least one agent that selectively inhibits or blocks the expression or activity of both CDK4 and/or CDK6, and ii) an immunotherapy, such that an immune response is upregulated in the subject, is provided.

As described above, numerous embodiments are further provided that can be applied to any aspect of the present invention and/or combined with any other embodiment described herein. For example, in one embodiment, the subject has a condition selected from the group consisting of a cancer, a viral infection, a bacterial infection, a protozoal infection, a helminth infection, asthma associated with impaired airway tolerance, and an immunosuppressive disease. In another embodiment, the condition is a cancer (e.g., breast cancer, colorectal cancer, etc.). In still another embodiment, at least some of the subject's immune cells, Tregs, or cancer cells express Rb and/or has functional Rb signaling. In yet another embodiment, at least some of the subject's immune cells, Tregs, or cancer cells have defective Rb expression and/or defective Rb signaling. In one embodiment, at least some of the subject's Tregs or cancer cells harbor genomic mutations causing defective Rb expression and/or defective Rb signaling. The condition described herein may be, e.g., resistant to immune checkpoint blockade. In one embodiment, the at least one agent increases the susceptibility to immune checkpoint blockade of the subject's cells, immune cells, Tregs, or cancer cells in the subject. In another embodiment, the at least one agent: a) increases the number of cancer infiltrating CD3+ T cells in the subject; b) increases antigen presentation by cancer cells in the subject; c) increases MHC class I expression by cancer cells in the subject; d) increases interferon production, signaling, and/or secretion by cancer cells in the subject; e) increases type III interferon production, signaling, and/or secretion by cancer cells in the subject; f) increases expression of at least one gene selected from the group consisting of STAT1, STAT2, IRF2, IRF6, IRF7, IRF9, NLRC5, OAS1, OAS2, IFIT1, IFIT2, IFIT6, BST2, SP100, RSAD2, CXCL9, CXCL10, CXCL11, Icam1, Vcam1, IL-29, IL-28a, IL-28b, ERV3-1, ERVK13-1, RIG-1, LGP2, and MDA5 by cancer cells in the subject; g) inhibits expression of at least one DNA methyltransferase (DNMT) by cancer cells in the subject; and/or h) inhibits expression of DNMT1 expression by cancer cells in the subject. In still another embodiment, the at least one agent is administered prior to administering the immunotherapy, optionally wherein the at least one agent is preadministered before subsequent administration of a combination of the at least one agent and the immunotherapy.

In one embodiment, the method further comprises administering one or more additional agents or therapies that upregulates an immune response. Such one or more additional agents or therapies may be, e.g., selected from the group consisting of a vaccine, chemotherapy, radiation, epigenetic modifiers, and targeted therapy. Such immunotherapy may be, e.g., selected from the group consisting of immune checkpoint inhibitor therapy, a sensitized antigen presenting cell, an oncolytic virus, an expression vector comprising an anticancer gene, and an inhibitor of a cancer antigen or a disease antigen. Such immune checkpoint inhibitor therapy may comprise, e.g., reducing or inhibiting the expression and/or function of an immune checkpoint molecule selected from the group consisting of CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR in the subject. In one embodiment, the immune checkpoint inhibitor therapy targets an immune checkpoint selected from the group consisting of PD-1, CTLA-4, PD-L1, PD-L2, and combinations thereof. In another embodiment, the at least one agent significantly reduces the number of Tregs in the spleen and/or lymph nodes of the subject. In still another embodiment, the at least one agent does not significantly reduce the number of Tregs in the thymus of the subject. In yet another embodiment, the at least one agent does not significantly affect differentiation of naïve CD4+ T cells into Tregs in the subject. In one embodiment, the at least one agent does not significantly affect Treg apoptosis in the subject. In another embodiment, the at least one agent does not significantly change the cell number of at least one cell type selected from the group consisting of B lymphocytes, natural killer cells, neutrophils, and monocytes. In still another embodiment, the at least one agent reduces the ratio of Tregs to CD3+ T cells and/or the ratio of Tregs to CD8+ T cells in the subject. In yet another embodiment, the Tregs comprise CD4+CD25+, CD4+FOXP3+, and/or CD4+CD25+FOXP3+ Tregs. In one embodiment, the at least one agent does not significantly modulate the number of CD8+ T cells and/or CD4+CD25− T cells. In another embodiment, the at least one agent reduces the expression of at least one marker selected from the group consisting of PD-1, TIM-3, CTLA-4, and LAG3 on the surface of CD4+ and/or CD8+ T cells. In still another embodiment, the at least one agent increases antigen presentation in the subject. In yet another embodiment, the at least one agent increases MHC class I expression in the subject. In one embodiment, the at least one agent increases T cell-mediated cytotoxicity in the subject. In another embodiment, the at least one agent increases interferon production, signaling, and/or secretion in the subject. In still another embodiment, the at least one agent increases type III interferon production in the subject. In yet another embodiment, the at least one agent increases expression of at least one gene selected from the group consisting of STAT1, STAT2, IRF2, IRF6, IRF7, IRF9, NLRC5, OAS1, OAS2, IFIT1, IFIT2, IFIT6, BST2, SP100, RSAD2, CXCL9, CXCL10, CXCL11, Icam1, Vcam1, IL-29, IL-28a, IL-28b, ERV3-1, ERVK13-1, RIG-1, LGP2, and MDA5 in the subject, or any ISG described herein, such as those listed in Table 2.

In one embodiment, the at least one agent inhibits at least one DNA methyltransferase (DNMT), such as DNMT1, in the subject. In another embodiment, the at least one agent does not significantly enhance senescence associated secretory phenotype (SASP) in the subject. In still another embodiment, the at least one agent is selected from the group consisting of: a small molecule CDK4 antagonist, a blocking intrabody or antibody that binds CDK4, a non-activating form of CDK4, a soluble form of an CDK4 natural binding partner, a CDK4 fusion protein, a nucleic acid molecule that blocks CDK4 transcription or translation, a small molecule CDK6 antagonist, a blocking intrabody or antibody that recognizes CDK6, a non-activating form of CDK6, a soluble form of a CDK6 natural binding partner, a CDK6 fusion protein, and a nucleic acid molecule that blocks CDK6 transcription or translation. In still another embodiment, said at least one agent comprises a small molecule (e.g., abemaciclib, palbociclib, and ribociclib that inhibits or blocks CDK4 and/or CDK6 expression or activity. In yet another embodiment, said at least one agent comprises an RNA interfering agent (e.g., a small interfering RNA (siRNA), small hairpin RNA (shRNA), microRNA (miRNA), or a piwiRNA (piRNA)) which inhibits or blocks CDK4 and/or CDK6 expression or activity. In one embodiment, said at least one agent comprises an antisense oligonucleotide complementary to CDK4 and/or CDK6. In another embodiment, said at least one agent comprises a peptide or peptidomimetic that inhibits or blocks CDK4 and/or CDK6 expression or activity. In still another embodiment, said at least one agent comprises an aptamer that inhibits or blocks CDK4 and/or CDK6 expression or activity. In yet another embodiment, said at least one agent is an intrabody or antibody, or an antigen binding fragment thereof, which specifically binds to CDK4 and/or CDK6. In one embodiment, said intrabody or antibody, or antigen binding fragment thereof, is murine, chimeric, humanized, or human. In another embodiment, said intrabody or antibody, or antigen binding fragment thereof, is detectably labeled, comprises an effector domain, comprises an Fc domain, and/or is selected from the group consisting of Fv, F(ab′)2, Fab′, dsFv, scFv, sc(Fv)2, and diabody fragments. In still another embodiment, said at least one agent described herein is administered in a pharmaceutically acceptable formulation. In yet another embodiment, the subject described herein is a mammal. Such mammal may be, e.g., an animal model of the condition or a human.

### BRIEF DESCRIPTION OF THE DRAWINGS

FIG. 1 includes 9 panels, identified as panels A, B, C, D, E, F, G, H, and I, which show that CDK4/6 inhibition induces tumor regression and increases antigen presentation. Panel A shows the average growth of MMTV-rtTA/tetO-HER2 tumors after treatment with indicated agents (n=17-22 tumors). Panel B shows an experimental schema for tumor gene expression analysis (n=11-12 tumors). Panel C shows gene ontology terms with adjusted p-values<0.05. Panel D shows relative expression of MHC Class I genes which were increased after treatment of CDK4/6 inhibitor abemaciclib. Panel E shows the qPCR result for antigen processing genes (n=12 per group, except n=6-8 for Erap1) which were increased after treatment of abemaciclib. Panel F shows the expression of genes upregulated by abemaciclib in two cancer cell lines after 7-day treatment (n=3). Panel G shows B2M/MHC I flow cytometry in cell lines. Left peaks in grey in each cytometry result (representative of two experiments) represents the FMO control. Panel H shows the ex vivo CD8+ T cell lysis of pre-treated primary MMTV-rtTA/tetO-HER2 tumor cells. Panel I shows the expression of various genes in TCGA samples (CCND1 diploid n=503; CCND1 amplified n=153). *p<0.05, **p<0.01, ***p<0.001, ****p<0.0001 (one-way ANOVA (panel A) and unpaired two-tailed t-tests (panel H) adjusted for multiple comparisons (panels D-F and I).

FIG. 2 includes 4 panels, identified as panels A, B, C, and D, which shows the tumor cell proliferation and expression of cell cycle related genes after CDK4/6 inhibition. Panel A shows the immunohistochemistry results for Ki-67 in MMTV-rtTA/tetO-HER2 tumors treated with abemaciclib or vehicle for 12 days. Representative images (scale bar=100 μm), and quantification of % Ki-67+ cells (n=7 tumors/group). Panels B-D show the relative expression of E2F transcription factors (Panel B), S phase genes (Panel C), and G2/M genes (Panel D) in MMTV-rtTA/tetO-HER2 tumors treated with abemaciclib for 12 days, compared to vehicle treatment, by transcriptomic analysis (n=11-12 tumors/group). *p<0.05, **p<0.01 (unpaired two-tailed t-tests adjusted for multiple comparisons when appropriate).

FIG. 3 includes 3 panels, identified as panels A, B, and C, which show that CDK4/6 inhibition enhances antigen presentation. Panels A-B show the analysis of gene expression changes in tumors from MMTV-rtTA/tetO-HER2 mice treated with vehicle or abemaciclib for 12 days as in Panel B of FIG. 2 (n=11-12 tumors). Specifically, panel A shows a Volcano plot of changes in gene expression. Red dots (e.g., dots above about 1.4 log₁₀p value) represent genes significantly altered compared to vehicle. Panel B shows GSEA terms significantly downregulated by abemaciclib compared to vehicle. Panel C shows the relative gene expression by qPCR in cell lines treated with DMSO or palbociclib for 7 days (unpaired two-tailed t tests adjusted for multiple comparisons). *p<0.05, **p<0.01, ***p<0.001.

FIG. 4 includes 4 panels, identified as panels A, B, C, and D, which show the effects of CDK4/6 inhibition on breast cancer cell proliferation and apoptosis in vitro. Panel A shows the relative numbers of breast cancer cells cultured in 250 nM (MDA-MB-453) or 500 nM (MDA-MB-361, BT474) abemaciclib for 11 days, followed by drug withdrawal. Panel B shows the representative SA-β-galactosidase staining of MDA-MB-453 cells (left) and BT474 cells (right) treated with DMSO or abemaciclib (MDA-MB-453, 250 nM; BT474, 500 nM) for 0, 4, and 7 days. Panel C shows the results of Western blotting with the indicated antibodies of cell lysates of SKBR3, BT474, MDA-MB-453, and MDA-MB-361 cells treated with DMSO, lapatinib, or abemaciclib for 48 hours. Panel D shows the results of Western blotting with the indicated antibodies of cell lysates of MDA-MB-453 cells pre-treated with DMSO or abemaciclib (500 nM) for 0, 1, or 7 days prior to exposure to staurosporine (500 nM) for 4 hours.

FIG. 5 includes 6 panels, identified as panels A, B, C, D, E, and F, which show that CDK4/6 inhibition stimulates interferon signaling. Panels A-C show genome-wide transcriptomic analysis of cell lines treated with DMSO or abemaciclib (500 nM) for 7 days. Specifically, Panel A shows top ranked GO terms in the analysis. Panel B shows the expression of multiple interferon-responsive transcription factors in two cancer cell lines. Panel C shows the expression of various interferon-sensitive genes in two cell lines (n=3). Panel D shows the protein levels of phospho- and total STAT1 in two cell lines after 0, 1, or 7-day treatment with abemaciclib, as detected by Western blot (using the same number of cells per lane). Panel E shows the relative expression of interferon-responsive transcription factors in MMTV-rtTA/tetO-HER2 tumors from Panel B of FIG. 1 (n=11-12 tumors). Panel F shows an immunofluorescent staining of tumors from Panel A of FIG. 1 (scale bar=100 μM) and quantification of nuclear STAT1. *p<0.01, ***p<0.001 (unpaired two-tailed t-tests for Panel F; adjusted for multiple comparisons for Panels B, C, and E).

FIG. 6 includes 6 panels, identified as panels A, B, C, D, E, and F, which shows that CDK4/6 inhibition increases interferon signaling. Panel A shows a relative expression of NLRC5 in MDA-MB-453 cells treated with DMSO or abemaciclib (500 nM, 7 days) (unpaired two-tailed t test). Panel B shows confirmation of p16-FLAG overexpression in MDA-MB-453 and BT474 cells (left) and gene expression in these cell lines by qPCR (right); (unpaired two-tailed t tests). Panels C-E show an analysis of gene expression in MMTV-rtTA/tetO-HER2 tumors from mice treated with vehicle or abemaciclib for 12 days, as in Panel B of FIG. 1. Specifically, Panel C shows the GSEA terms upregulated by abemaciclib as compared to vehicle. Panel D shows the relative expression of interferon-responsive T cell chemoattractants. Panel E shows the relative expression of multiple interferon-sensitive genes (ISGs). Panel F shows the correlation of relative expression of Stat1 and Nlrc5 with genes involved in antigen processing and presentation in MMTV-rtTA/tetO-HER2 tumors. Blue dots, vehicle-treated tumors; red dots, abemaciclib-treated tumors. (r is Pearson product-moment correlation coefficient). *p<0.05, ***p<0.001.

FIG. 7 includes 6 panels, identified as panels A, B, C, D, E, and F, which show that CDK4/6 inhibitors induce viral mimicry and type III interferon expression. Panel A shows the effect of neutralization of IFN-γ or IFN-α on relative STAT1 mRNA expression in MDA-MB-453 cells by qPCR (unpaired two-tailed tests adjusted for multiple comparisons). Panel B shows the impact of neutralization of IFN-α on phospho-STAT1 and total STAT1 protein in indicated cell lines. Protein from the same number of cells was used per lane. Panel C shows the effect of neutralization of IFN-γ on phospho-STAT1 and STAT1 total protein in indicated cell lines. Protein from the same number of cells was used per lane. Panel D shows the relative expression of type III interferon genes in MDA-MB-453 cells treated with abemaciclib (500 nM) for 7 days compared to DMSO (unpaired two-tailed t-test). Panel E shows the relative DNMT3A expression by transcriptome analysis of MDA-MB-453 and MDA-MB-361 cells treated with abemaciclib (500 nM) for 0 h, 24 h, 48 h, or 7 days. Panel F shows the relative mRNA expression of ERVs in indicated cells treated with abemaciclib (500 nM, 7 days) (unpaired two-tailed t-test). *p<0.05, **p<0.01, ***p<0.001, ****p<0.0001.

FIG. 8 includes 6 panels, identified as panels A, B, C, D, E, and F, which show that CDK4/6 inhibitors suppress DNMT1 expression to induce viral mimicry. Panels A-B show the expression of Type III interferons in indicated cell line in conditioned media after treatment with DMSO or abemaciclib (500 nM, 7 days). Representative data of two independent experiments are shown. Panel C shows the phospho- and total STAT1 protein levels in MDA-MB-453 cells treated with abemaciclib+/−ruxolitinib for 7 days. Protein from the same number of cells was used per lane. Panel D shows the relative DNMT1 expression in indicated cell lines after treatment with abemaciclib (n=3). Panel E shows the relative expression of Dnmt1 in MMTV-rtTA/tetO-HER2 tumors from Panel B of FIG. 1, p=0.05 and n=11-12 tumors. Panel F shows the relative expression of cytosolic pattern recognition receptors in the indicated cell lines after 7-day treatment (n=3). *p0.05, **p<0.01, ***p<0.001, ****p<0.0001 (unpaired two-tailed t-tests (for panels A, B, and E), and adjusted for multiple comparisons (for panels D and F).

FIG. 9 includes 4 panels, identified as panels A, B, C, and D, which show that abemaciclib induces a “senescence-like” phenotype without evidence of senescence associated secretory phenotype (SASP). Panel A shows the representative SA-β-galactosidase staining (left) of MMTV-rtTA/tetO-HER2 tumors treated with vehicle or abemaciclib for 12 days (scale bar=500 μm). Quantification of relative SA-β-galactosidase positive area is also shown (right). Panel B shows the relative mRNA expression of SASP factors in MMTV-rtTA/tetO-HER2 tumors treated as in Panel A of FIG. 1. The relative IL6 expression was determined by qPCR and Il1a and Il1b by transcriptome. Panel C compares IL6 expression (detected by qPCR) in MDA-MB-453 and BT474 cells treated with DMSO or abemaciclib (500 nM) for 7 days. Panel D shows the relative mRNA expression of IL6 upon doxorubicin-induced senescence. MDA-MB-453 and BT474 cells were treated with doxorubicin (200 nM) for 24 h, and mRNA extracted 3 days later for qPCR (unpaired two-tailed t tests). **p<0.01.

FIG. 10 includes 9 panels, identified as panels A, B, C, D, E, F, G, H, and I, which show the impact of CDK4/6 inhibition on immune cell populations and Treg biology. Panel A shows the flow cytometric analysis of immune populations in MMTV-rtTA/tetO-HER2 tumors treated with vehicle or abemaciclib for 12 days (n=15-17 tumors/group). Panel B shows plasma autoantibodies in tumor-free and tumor-bearing mice treated with vehicle or abemaciclib for 12 days (n=6-8 mice/group). For panels C-G, tumor-free FVB mice were treated with vehicle or abemaciclib for 12 days. Panel C compares thymic mass with or without abemaciclib/palbociclib treatment. Thymic cell populations were quantified by flow cytometry. Panel D compares the percentage of CD4+CD8+ double positive (DP) thymocytes with or without abemaciclib/palbociclib treatment. Panel E compares the percentage of CD4+ single positive (SP) thymocytes with or without abemaciclib/palbociclib treatment. Panel F compares the percentage of CD8+ single positive (SP) thymocytes with or without abemaciclib/palbociclib treatment. Panel G compares the percentage of CD4+FoxP3+ regulatory T cells with or without abemaciclib/palbociclib treatment (one-way ANOVA, n=4-5 mice/group). Panel H shows the effect of DMSO or abemaciclib on ex vivo differentiation of CD4+CD25− T cells into Tregs in the presence of TGF-β for 72 hours. Panel I shows the effect of DMSO or abemaciclib treatment for 72 hours on Treg apoptosis measured by Annexin V staining. *p<0.05, **p<0.01, ***p<0.001.

FIG. 11 includes 18 panels, identified as panels A, B, C, D, E, F, G, H, I, J, K, L, M, N, O, P, Q, and R, which show that CDK4/6 inhibition increases CD3+ T cells while inhibiting Treg proliferation. Panels A-B show the percentage of T cells (Panel A) and Tregs (CD4+FoxP3+) (Panel B), quantified by flow cytometry of MMTV-rtTA/tetO-HER2 tumors treated as indicated for 12 days (Mann-Whitney test, n=15-17 tumors). Panel C shows the quantification of Treg:CD3 ratio in tumors from Panel A of FIG. 1 (Mann-Whitney test). Panel D shows the levels of Tregs in peripheral blood of MMTV-rtTA/tetO-HER2 mice treated with the indicated agents (unpaired two-tailed t-test, n=4 mice/group). Panels E-F show the levels of Tregs in spleens (Panel E) and lymph nodes (LN) (Panel F) of tumor-free FVB mice with or without inhibitor treatment (one-way ANOVA corrected for multiple comparisons, n=7-8 mice/group). Panel G compares the proliferation of CD4+CD25-, CD8+, and CD4+CD25+ T cells in vitro with abemaciclib treatment. Data representative of two independent experiments are shown (two-way ANOVA corrected for multiple comparisons). Panels H-I show the percentage of Ki-67+ T cell populations in the spleens (Panel H) and lymph nodes (Panel I) of tumor-free FVB mice treated for 12 days with indicated agents (two-way ANOVA corrected for multiple comparisons, n=7-8 mice/group). Panel J shows immunofluorescence images of tumors from Panel A of FIG. 1 (scale bar=100 μm) and quantification (unpaired two-tailed t-test; n=7 tumors/group). Panel K compares Treg numbers in the tumor (left) and blood (right) of mice bearing CT-26 colorectal carcinomas. Panel L compares CD8+ T cells in proliferation in MMTV-rtTA/tetO-HER2 tumors with or without inhibitor treatment. Double staining of the tumor showed no significant reduction in the number of Ki67+CD8+ T cells after 12-day treatment of abemaciclib, indicating that CD8+ T cell proliferation is not significantly suppressed by CDK4/6 inhibitors. Panel M compares the ratio of Treg numbers vs. CD8+ T cell numbers in MMTV-rtTA/tetO-HER2 tumors with or without inhibitor treatment. Panel N shows a comparison of the Treg:CD8+T ratios in the blood, lymph node (LN), and spleen (spl) of mice bearing CT-26 tumors with or without treatment. Panel O shows that the reduction in the Treg:CD8+ T cells in Panel N is tumor-independent. Specifically, such ratios in the spleen (spl, left) and the lymph node (LN) of tumor-free FVB mice after 12-day of treatment of CDK4/6 inhibitors were compared. Panel P shows that CDK4/6 inhibition selectively suppresses Dnmt1 expression in Tregs. Tumor-free mice were treated with abemaciclib or vehicle for 12 days. T cell subsets were sorted from spleens and lymph nodes and qPCR was performed for Dnmt1. Panel Q shows that DNMT1 suppression in Tregs is associated with increased expression of CDKN1A. Tumor-free mice were treated with abemaciclib or vehicle for 12 days. T cell subsets were sorted from spleens and lymph nodes and qPCR was performed for Cdkn1a analysis. Panel R shows an exemplary mechanism for suppression of Treg proliferation by CDK4/6 inhibitors. *p0.05, **p<0.01, ***p<0.001, ****p<0.0001.

FIG. 12 includes 2 panels, identified as panels A and B, which show that abemaciclib causes tumor growth delay, but not regression, in nude mice. Panel A shows the average growth of MMTV-rtTA/tetO-HER2 tumors implanted into athymic mice treated with vehicle or abemaciclib (one-way ANOVA, n=10 tumors/group). Panel B shows the immunohistochemistry of tumors in panel A for Ki-67. Representative images were shown (left). Quantification of percentage of Ki-67+ cells (right) was analyzed by unpaired two-tailed t-test (scale bar=100 μm) ***p<0.001, ****p<0.0001.

FIG. 13 includes 7 panels, identified as panels A, B, C, D, E, F, and G, which show that CDK4/6 inhibition mediates CD8+ T cell-dependent tumor regression and enhances response to checkpoint blockade. Panel A shows the maximum fold change in tumor volume after treatment with abemaciclib with or without a CD8 neutralizing antibody (unpaired two-tailed t test). Panels B-C show the inhibitory co-receptor expression on CD8+ T cells in MMTV-rtTA/tetO-HER2 tumors treated for 6 days (two-way ANOVA corrected for multiple comparisons, n=5-6 tumors/group). Panel D shows the relative Ifng expression by qPCR in tumors from Panel A of FIG. 1 (unpaired two-tailed t test, n=10 tumors/group). Panel E shows an experimental scheme to treat MMTV-rtTA/tetO-HER2 tumor-bearing mice with either vehicle or abemaciclib, as well as a control IgG or an anti-PD-L1 antibody. Panel F compares the average tumor growth after treatments in Panel E (one-way ANOVA, n=18-19 tumors/group). Panel G shows that CDK4/6 inhibition promotes tumor immunogenicity and an anti-tumor immune response through direct effects on tumor cells and the immune milieu. *p<0.05, **p<0.01, ***p<0.001, ****p<0.0001.

FIG. 14 includes 11 panels, identified as panels A, B, C, D, E, F, G, H, I, J, and K, which show the effects of abemaciclib on T cell exhaustion. Panel A shows the validation of CD8+ T cell depletion prior to start of abemaciclib. Forty-eight hours after commencing treatment with CD8 neutralizing antibodies, absolute number (left) and percentage (right) of CD8+ T cells in peripheral blood were determined by flow cytometry (unpaired two-tailed t-tests). Panels B-E compare the expression of inhibitory co-receptors on intratumoral CD8+ T cells in MMTV-rtTA/tetO-HER2 tumors after 6 days of treatment with abemaciclib or vehicle. Panel B shows PD-1 cell-surface expression by representative flow cytometry plots (left) and quantifications with analysis by two-way ANOVA corrected for multiple comparisons (right). Panels C-D compare representative flow cytometry plots for CTLA-4 (Panel C) and LAG3 (Panel D). Panel E shows the quantification of Panels C-D, as analyzed by two-way ANOVA corrected for multiple comparisons. Panels F-K compare the expression of inhibitory co-receptors on intratumoral CD4+ T cells in tumors described in Panels B-E. Representative flow cytometry plots are shown for PD-1 (Panel F), Tim-3 (Panel G), CTLA-4 (Panel H), and LAG3 (Panel I). Panel J shows the quantification of Panels F-I by two-way ANOVA corrected for multiple comparisons. Panel K shows the quantification of number of inhibitory receptors per cell. **p<0.01, ***p<0.001, ****p<0.0001.

FIG. 15 includes 4 panels, identified as panels A, B, C, and D, which summarize the data described in Examples 1-8.

Note that for every figure containing a histogram, the bars from left to right for each discreet measurement correspond to the figure boxes from top to bottom in the figure legend as indicated.

### DETAILED DESCRIPTION OF THE INVENTION

Tumors evade the immune system through several mechanisms, including impaired antigen presentation. Indeed, defects in the interferon signaling pathway and downstream transcription factors foster immune evasion and resistance to immune checkpoint blockade. It has been determined herein that CDK4/6 inhibitors, previously thought as inducing cancer cell cycle arrest, induces an anti-tumor immune response by a combination of two phenomena: enhanced antigen presentation by tumor cells and a re-programming of the immune suppressive microenvironment. Surprisingly, CDK4/6 inhibitors specifically reduce the number of circulating regulatory T cells (Tregs) in a subject. For example, the Tregs in the spleen and/or lymph nodes, but not in the thymus, can be significantly reduced by CDK4/6 inhibitors. In addition, other types of T cells remain unchanged. Such reduction of circulating Tregs is caused by suppression of DNA methyltransferase (DNMT) levels, such as DNMT1, in Tregs, which further enhances their cell cycle arrest. Thus, directly against previous concerns that CDK inhibitors might render immune checkpoint therapy less effective due to T cell cycle inhibition (Sherr (2016) N Engl J Med 375:1920-1923), the results provided herein demonstrate that CDK4/6 inhibitors promote anti-tumor immunity and can be used alone or in combination with immune checkpoint therapy to treat cancers.

Accordingly, the present invention relates, in part, to methods of selectively reducing the number of circulating regulatory T cells (Tregs) in a subject with at least one CDK4/6 inhibiting agent, alone or in combination with an immunotherapy, such as an immune checkpoint inhibitor therapy. In another aspect, the present invention provides methods of upregulating an immune response in a subject with a combination of at least one CDK4/6 inhibiting agent and an immunotherapy, such as an immune checkpoint inhibitor therapy.

### I. Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

The term “altered amount” or “altered level” refers to increased or decreased copy number (e.g., germline and/or somatic) of a biomarker nucleic acid, e.g., increased or decreased expression level in a cancer sample, as compared to the expression level or copy number of the biomarker nucleic acid in a control sample. The term “altered amount” of a biomarker also includes an increased or decreased protein level of a biomarker protein in a sample, e.g., a cancer sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal and/or amount of the biomarker, if the amount of the biomarker is greater or less, respectively, than the normal or control level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternatively, the amount of the biomarker in the subject can be considered “significantly” higher or lower than the normal and/or control amount if the amount is at least about two, and preferably at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, two times, three times, four times, five times, or more, or any range in between, such as 5%-100%, higher or lower, respectively, than the normal and/or control amount of the biomarker. Such significant modulation values can be applied to any metric described herein, such as altered level of expression, altered activity, changes in cancer cell hyperproliferative growth, changes in cancer cell death, changes in biomarker inhibition, changes in test agent binding, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least twice, and more preferably three, four, five or ten or more times the expression level or copy number of the biomarker in a control sample (e.g., sample from a healthy subject not having the associated disease) and preferably, the average expression level or copy number of the biomarker in several control samples.

The term “altered activity” of a biomarker refers to an activity of the biomarker which is increased or decreased in a disease state, e.g., in a cancer sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

Unless otherwise specified here within, the terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, biomarker peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “assigned score” refers to the numerical value designated for each of the biomarkers after being measured in a patient sample. The assigned score correlates to the absence, presence or inferred amount of the biomarker in the sample. The assigned score can be generated manually (e.g., by visual inspection) or with the aid of instrumentation for image acquisition and analysis. In certain embodiments, the assigned score is determined by a qualitative assessment, for example, detection of a fluorescent readout on a graded scale, or quantitative assessment. In one embodiment, an “aggregate score,” which refers to the combination of assigned scores from a plurality of measured biomarkers, is determined. In one embodiment the aggregate score is a summation of assigned scores. In another embodiment, combination of assigned scores involves performing mathematical operations on the assigned scores before combining them into an aggregate score. In certain, embodiments, the aggregate score is also referred to herein as the “predictive score.”

The term “biomarker” includes a measurable entity of the present invention that has been determined to be predictive, either alone or in combination, of response of a cancer to one or more inhibitors of CDK4 or CDK6, either alone or in combination with an immunotherapy. Biomarkers can include, without limitation, nucleic acids and proteins, including those shown in Table 1, the Examples, and the Figures. Biomarkers include markers listed herein which are useful in the diagnosis of cancer and/or sensitivity to anti-cancer treatments thereof, e.g., over- or under-activity, emergence, expression, growth, remission, recurrence or resistance of tumors before, during or after therapy. The predictive functions of the marker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression (e.g., by ISH, Northern Blot, or qPCR), increased or decreased protein level (e.g., by IHC), or increased or decreased activity (determined by, for example, modulation of a pathway in which the marker is involved), e.g., in more than about 5%, 6%, 7%, 8%, 9%, 10%, 11%, 12%, 13%, 14%, 15%, 20%, 25%, or more of human cancers types or cancer samples; (2) its presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its presence or absence in clinical subset of subjects with cancer (e.g., those responding to a particular therapy or those developing resistance). Biomarkers also include “surrogate markers,” e.g., markers which are indirect markers of cancer progression. The term “biomarker” also include markers listed herein which are useful in the analysis of the effects of anti-cancer treatments, such as the size of the tumor, the proliferation and/or metastasis rate of cancer cells, the number of cancer cells, the life span of the subject having the cancer, etc. Biomarkers also include markers listed herein in cell signaling pathways, such as the number of Treg and/or other T cells, the differentiation rate and/or the apoptosis/cytotoxicity rate of various T cells or other immune cells, the expression of various proteins expressed on the cell surface of T cells or other immune cells, the antigen presentation efficacy, the production of various signal proteins (e.g., interferons) and their responsive genes, DNA methylation and transcription efficacy, senescence/proliferation status, etc.

The term “CDK4” refers to Cyclin Dependent Kinase 4, as a member of the Ser/Thr protein kinase family. This protein is highly similar to the gene products of S. cerevisiae cdc28 and S. pombe cdc2. It is a catalytic subunit of the protein kinase complex that is important for cell cycle G1 phase progression. The activity of CDK4 is restricted to the G1-S phase, which is controlled by the regulatory subunits D-type cyclins and CDK inhibitor p16INK4a. CDK4 is also responsible for the phosphorylation of retinoblastoma gene product (Rb) (Hanks et al. (1987) Proc. Natl. Acad. Sci. USA 84:388-392; Mitchell et al. (1995) Chrom. Res. 3:261-262; Medema et al. (1995) Proc. Natl. Acad. Sci. USA 92:8871-8875; Hall et al. (1995) Oncogene 11:1581-1588). The cyclin D-CDK4 (DC) complexes phosphorylate and inhibit members of the retinoblastoma (Rb) protein family, including Rb1, and regulate the cell-cycle during G1/S transition. Phosphorylation of Rb1 allows dissociation of the transcription factor E2F from the RB/E2F complexes and the subsequent transcription of E2F target genes which are responsible for the progression through the G1phase. Cyclin D-CDK4 complexes are major integrators of various mitogenic and antimitogenic signals. CKD4 also phosphorylates SMAD3 in a cell-cycle-dependent manner and represses its transcriptional activity (Matsuura et al. (2004) Nature 430:226-231). CDK4 is also a component of a ternary complex, cyclin D/CDK4/CDKN1B, which is required for nuclear translocation and activity of the cyclin D-CDK4 complex.

The term “CDK4” is intended to include fragments, variants (e.g., allelic variants), and derivatives thereof. Representative human CDK4 cDNA and human CDK4 protein sequences are well-known in the art and are publicly available from the National Center for Biotechnology Information (NCBI). For example, a human CDK4 transcript sequence is available as NM_000075.3 and human CDK4 amino acid sequence is available as NP_000066.1. Human CDK4 protein has 303 amino acids and around ˜33,730 Da of molecular mass. Human CDK4 has a Serine/Threonine protein kinase catalytic domain (S/T Kc) (amino acid residue no. 6-295 of SEQ ID NO:2), which includes a region required for binding D-type cyclines (amino acid residue no. 50-56 of SEQ ID NO:2). Nucleic acid and polypeptide sequences of CDK4 orthologs in organisms other than humans are well-known and include, for example, chimpanzee CDK4 (XM_509173.5, XP_509173.2, XM_009425031.1, and XP_009423306.1), monkey CDK4 (XM_001116422.2 and XP_001116422.1), dog CDK4 (XM_844780.3 and XP_849873.1), cow CDK4 (NM_001037594.2 and NP_001032683.1), mouse CDK4 (NM_009870.3 and NP_034000.1), rat CDK4 (NM_053593.2 and NP_446045.1), frog CDK4 (NM_001016742.1 and NP_001016742.1), and zebrafish CDK4 (NM_001077777.1 and NP_001071245.1). It is to be noted that the term can further be used to refer to any combination of features described herein regarding CDK4 molecules. For example, any combination of sequence composition, percentage identify, sequence length, domain structure, functional activity, etc. can be used to describe an CDK4 molecule for use in the present invention.

As used herein, “CDK4/6 inhibitors,” “CDK4 inhibitors,” and “CDK6 inhibitors,” generally refer to a large group of compounds having the ability to selectively inhibit one or more of the recited cyclin-dependent kinases (CDK), such as CDK4 and CDK6. Such inhibitors selectively bind to the well-known CDK4, and CDK6 cyclin-dependent kinases, and prevent phosphorylation of the retinoblastoma (Rb) protein to thereby stop cell cycle progression by inducing a cell cycle hold at the G1 phase. Such inhibitors need not be specific for a given CDK member, so long as they selectively inhibit a desire CDK protein with relatively minimal off-target effects on non-CDK proteins. Other inhibitors may selectively bind to the well-known substrate(s) (e.g., Rb, SMAD3, etc.) and/or other binding partner(s) (e.g., cyclin D, CDKN1B, etc.) of CDK4 and/or CDK6 and thus inhibit CDK4/6 function. Numerous representative examples of such inhibitors having a variety of chemical structures are well-known in the art. For example, CDK4 and CDK6 inhibitors include abemaciclib (previously as LY2835219, by Eli Lilly, for breast cancers), ribociclib (previously as LEE 011, an inhibitor of cycline D1/CDK4 and CDK6, by Novartis and Astex Pharmaceuticals, approved by FDA for use in combination with an aromatase inhibitor to treat metastatic breast cancers), palbociclib (previously as PD-0332991, a highly-specific CDK4/6 inhibitor, by Pfizer for the treatment of ER-positive and HER2-negative breast cancers); P-276-00 (a selective inhibitor of CDK4-cyclin D1, under development by Nicholas Piramal for the treatment of cancer); GW-491619 (a CDK4 inhibitor, under development by GlaxoSmithKline for the treatment of cancer); NU-6027 (a cyclin dependent kinase (CDK) inhibitor under investigation by AstraZeneca for use in cancer); AG-12275 (a selective CDK4 inhibitor under investigation by Pfizer for the treatment of cancer); AG-12286 (a broad-spectrum CDK4 inhibitor under investigation by Pfizer for the treatment of cancer); PD-0166285 (a cyclin A-mediated inhibitor of CDK4 under investigation by Pfizer for the treatment of cancer); and Alvocidib (flavopiridol; HMR-1275, an inhibitor of Cdk4 under development by Sanofi-Aventis as an anticancer agent). Other CDK4/6 inhibitors are described in, for example, WO 03/062236, and representative examples include 8-Cyclopentyl-2-(pyridin-2-ylamino)-8H-pyrido[2,3-d]pyrimidin-7-one; 6-Bromo-8-cyclopentyl-2-(5-piperazin-1-yl-pyridin-2-ylamino)-8H-pyrido[2,3-d]pyrimidin-7-one hydrochloride; 8-Cyclopentyl-6-ethyl-2-(5-piperazin-1-yl-pyridin-2-ylamino)-8H-pyrido[2,3-d]pyrimidin-7-one hydrochloride; and the like. In addition, CDK4 inhibitors can be prepared based on the descriptions found in U.S. Pat. No. 6,689,864, PCT Patent Publication No. WO08/007123, PCT Patent Publication No. WO07/140222, PCT Patent Publication No. WO06/106046, PCT Patent Publication No. WO03/062236, PCT Patent Publication No. WO05/005426, PCT Patent Publication No. WO99/21845; PCT Patent Publication No. WO06/097449, PCT Patent Publication No. WO06/097460, PCT Patent Publication No. WO99/02162, and PCT Patent Publication No. WO99/50251. Commercially available CDK4 inhibitors also include Arcyriaflavin A (Cat #2457), NSC 625987 (Cat #2152), Ryuvidine (Cat #2609), and others from Tocris Bioscience (Bristol, UK). Standard assays for analyzing CDK4, and CDK6 inhibition and activity are well-known in the art (see, e.g., Fry et al. (2001) J. Biol. Chem. 276:16617-16623). Anti-CDK4 antibodies are also well-known in the art, including, at least, AM05224PU-N and other antibodies from OriGene (Rockville, Md.), Cat # AF5254 from R&D Systems (Minneapolis, Minn.), Cat # s 12790 and 42749 from Cell Signaling Technology (Danvers, Mass.), Cat # sc-23896 and others from Santa Cruz Biotechnology (Dallas, Tex.), etc. miRNA/siRNA/shRNA products for CDK4 are also well-known in the art, including, at least, Cat # sc-29261, sc-29262, and others from Santa Cruz Biotechnology. CRISPR knockout products for CDK4 are also well-known in the art, including, at least, Cat # KN303041 from OriGene.

The term “CDK6” refers to Cyclin Dependent Kinase 6, as a member of the cyclin-dependent protein kinase (CDK) family. This kinase is a catalytic subunit of the protein kinase complex that is important for cell cycle G1 phase progression and G1/S transition. The activity of this kinase first appears in mid-G1 phase, which is controlled by the regulatory subunits including D-type cyclins and members of INK4 family of CDK inhibitors. This kinase, as well as CDK4, has been shown to phosphorylate, and thus regulate the activity of, tumor suppressor protein Rb. Expression of this gene is up-regulated in some types of cancer. The CDK6 gene is conserved in eukaryotes, including the budding yeast and the nematode Caenorhabditis elegans. The CDK6 gene is located on chromosome 7 in humans. The gene spans 231,706 base pairs and encodes a 326 amino acid protein. The gene is overexpressed in cancers like lymphoma, leukemia, medulloblastoma and melanoma associated with chromosomal rearrangements. The CDK6 protein contains a catalytic core composed of a serine/threonine domain (Reinhardt and Yaffe (2013) Nature Reviews Molecular Cell Biology 14:563-580). This protein also contains an ATP-binding pocket, inhibitory and activating phosphorylation sites, a PSTAIRE-like cyclin-binding domain and an activating T-loop motif (Lim and Kaldis (2013) Development 140:3079-3093). After binding the Cyclin in the PSTAIRE helix, the protein changes its conformational structure to expose the phosphorylation motif. The protein can be found in the cytoplasm and the nucleus, however most of the active complexes are found in the nucleus of proliferating cells.

The term “CDK6” is intended to include fragments, variants (e.g., allelic variants), and derivatives thereof. Representative human CDK6 cDNA and human CDK6 protein sequences are well-known in the art and are publicly available from the National Center for Biotechnology Information (NCBI). For example, human CDK6 transcript sequences are available as NM_001259.6 (the longer transcript) and NM_001145306.1 (the shorter transcript differing in the 5′ UTR compared to the longer transcript, encoding the same protein) and human CDK6 amino acid sequence is available as NP_001250.1. Human CDK6 protein has 326 amino acids and around 36,938 Da of molecular mass. Human CDK6 has a Serine/Threonine protein kinase catalytic domain (S/T Kc) (amino acid residue no. 11-300 of SEQ ID NO:7), which includes multiple phosphorylation sites at amino acid residue nos. 13, 24, 49, 70, 177, and 325 of SEQ ID NO:7, an acetylation site at amino acid residue no. 264 of SEQ ID NO:7, and an activation loop (A-loop) region (amino acid residue no. 162-184 of SEQ ID NO:7). Nucleic acid and polypeptide sequences of CDK6 orthologs in organisms other than humans are well-known and include, for example, chimpanzee CDK6 (XM_003318579.3 and XP_003318627.1, XM_001167181.3 and XP_001167181.1, XM_009453611.2 and XP_009451886.1, and XM_016957767.1 and XP_016813256.1), rhesus monkey CDK6 (NM_001261307.1 and NP_001248236.1), dog CDK6 (XM_014118897.1 and XP_013974372.1), cattle CDK4 (NM_001192301.1 and NP_001179230.1), mouse CDK6 (NM_009873.3 and NP_034003.1), rat CDK6 (NM_001191861.1 and NP_001178790.1), chicken CDK6 (NM_001007892.2 and NP_001007893.1), frog CDK4 (XM_002934591.4 and XP_002934637.2, and XM_012965611.2 and XP_012821065.1), and zebrafish CDK6 (NM_001144053.1 and NP_001137525.1). It is to be noted that the term can further be used to refer to any combination of features described herein regarding CDK6 molecules. For example, any combination of sequence composition, percentage identify, sequence length, domain structure, functional activity, etc. can be used to describe an CDK6 molecule for use in the present invention.

As used herein, “CDK4/6 inhibitors,” “CDK4 inhibitors,” and “CDK6 inhibitors,” generally refer to a large group of compounds having the ability to selectively inhibit one or more of the recited cyclin-dependent kinases (CDK), such as CDK4 and CDK6. Such inhibitors selectively bind to the well-known CDK4, and CDK6 cyclin-dependent kinases, and prevent phosphorylation of the retinoblastoma (Rb) protein to thereby stop cell cycle progression by inducing a cell cycle hold at the G1 phase. Such inhibitors need not be specific for a given CDK member, so long as they selectively inhibit a desire CDK protein with relatively minimal off-target effects on non-CDK proteins. Other inhibitors may selectively bind to the well-known substrate(s) (e.g., Rb, SMAD3, etc.) and/or other binding partner(s) (e.g., cyclin D, CDKN1B, etc.) of CDK4 and/or CDK6 and thus inhibit CDK4/6 function. Numerous representative examples of such inhibitors having a variety of chemical structures are well-known in the art. For example, CDK6 inhibitors include abemaciclib (previously as LY2835219, by Eli Lilly, for breast cancers), ribociclib (previously as LEE 011, an inhibitor of cycline D1/CDK4 and CDK6, by Novartis and Astex Pharmaceuticals, approved by FDA for use in combination with an aromatase inhibitor to treat metastatic breast cancers), palbociclib (previously as PD-0332991, a highly-specific CDK4/6 inhibitor, by Pfizer for the treatment of ER-positive and HER2-negative breast cancers), Fisetin from Tocris Bioscience (Bristol, UK), and others disclosed above, such as CDK4/6 inhibitors are described in, for example, WO 03/062236, and representative examples include 8-Cyclopentyl-2-(pyridin-2-ylamino)-8H-pyrido[2,3-d]pyrimidin-7-one; 6-Bromo-8-cyclopentyl-2-(5-piperazin-1-yl-pyridin-2-ylamino)-8H-pyrido[2,3-d]pyrimidin-7-one hydrochloride; 8-Cyclopentyl-6-ethyl-2-(5-piperazin-1-yl-pyridin-2-ylamino)-8H-pyrido[2,3-d]pyrimidin-7-one hydrochloride; and the like. In addition, CDK4 inhibitors can be prepared based on the descriptions found in U.S. Pat. No. 6,689,864, PCT Patent Publication No. WO08/007123, PCT Patent Publication No. WO07/140222, PCT Patent Publication No. WO06/106046, PCT Patent Publication No. WO03/062236, PCT Patent Publication No. WO05/005426, PCT Patent Publication No. WO99/21845; PCT Patent Publication No. WO06/097449, PCT Patent Publication No. WO06/097460, PCT Patent Publication No. WO99/02162, and PCT Patent Publication No. WO99/50251. Standard assays for analyzing CDK4, and CDK6 inhibition and activity are well-known in the art (see, e.g., Fry et al. (2001) J Biol. Chem. 276:16617-16623). Anti-CDK4 antibodies are also well-known in the art, including, at least, AM05226PU-N and other antibodies from OriGene (Rockville, Md.), Cat # H00001021-M01 and others from Novus Biologicals (Littleton, Colo.), Cat # s 13331 and 3136 from Cell Signaling Technology (Danvers, Mass.), Cat # ab124821 and others from abcam (Cambridge, Mass.), Cat # sc-7961 and others from Santa Cruz Biotechnology (Dallas, Tex.), etc. miRNA/siRNA/shRNA products for CDK4 are also well-known in the art, including, at least, Cat # sc-29264, sc-35048, and others from Santa Cruz Biotechnology. CRISPR knockout products for CDK4 are also well-known in the art, including, at least, Cat # sc-400309 and sc-419605 from Santa Cruz Biotechnology and the CRISPR guide RNA products for CDK6 from GenScript (Piscataway, N.J.).

The term “T cell” includes, e.g., CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

As used herein, “Treg(s)” refers to regulatory T-cells, which are naturally occurring CD4+CD25+FOXP3+T lymphocytes that comprise ˜5-10% of the circulating CD4+ T cell population, act to dominantly suppress autoreactive lymphocytes, and control innate and adaptive immune responses (Piccirillo and Shevach (2004) Semin. Immunol. 16:81-88; Fehervari and Sakaguchi (2004) Curr. Opin. Immunol. 16:203-208; Azuma et al. (2003) Cancer Res. 63:4516-4520; Cederbom et al. (2000) Eur. J Immunol. 30:1538-1543; Maloy et al. (2003) J. Exp. Med. 197:111-119; Serra et al. (2003) Immunity 19:877-889; Thornton and Shevach (1998) J Exp. Med. 188:287-296; Janssens et al. (2003) J. Immunol. 171:4604-4612; Gasteiger et al. (2013) J. Exp. Med. 210:1167-1178; Sitrin et al. (2013) J. Exp. Med. 210:1153-1165). Tregs achieve this suppressing, at least in part, by inhibiting the proliferation, expansion, and effector activity of conventional T cells (Tcons). They also suppress effector T cells from destroying their (self-)target, either through cell-cell contact by inhibiting T cell help and activation, or through release of immunosuppressive cytokines such as IL-10 or TGF-β. Depletion of T_{reg} cells was shown to enhance IL-2 induced anti-tumor immunity (Imai et al. (2007) Cancer Sci. 98:416-23).

Conventional T cells, also known as Tconv or Teffs, have effector functions (e.g., cytokine secretion, cytotoxic activity, and the like) to increase immune responses by virtue of their expression of one or more T cell receptors. Tcons are defined as any T cell population that is not a Treg and include, for example, naïve T cells, activated T cells, memory T cells, resting Tcons, or Tcons that have differentiated toward, for example, the Th1 or Th2 lineages. Thus, increasing the number of Tregs, increasing Treg activity, and/or decreasing Treg cell death (e.g., apoptosis) is useful for suppressing unwanted immune reactions associated with a range of immune disorders (e.g., cGVHD). For example, in a murine model a 1:1 mix of CD4+CD25+ Tregs and CD25− effector T cells added to donor bone marrow stem cells suppressed alloimmune activation and GVHD without increasing malignant relapse post-transplant (Edinger et al. (2003) Nat. Med. 9:1144-1150). In humans, impaired Treg reconstitution in HSCT recipients occurs with active cGVHD (Zorn et al. (2005) Blood 106:2903-2911). In participants with active cGVHD, impaired Tregs reconstitution, low levels of telomerase, and shortened telomeres, are believed to contribute to decreased survival of Tregs (Zorn et al. (2005) Blood 106:2903-2911; Matsuoka et al. (2010) J. Clin. Invest. 120:1479-1493; Kawano et al. (2011) Blood 118:5021-5030). The role of IL-2 in Tregs homeostasis and function is believed to account for its limited efficacy as an anti-immune disorder therapy, and explain in part the finding that in vivo administration of IL-2 plus syngeneic T-cell-depleted donor marrow prevents GVHD after MHC-mismatched murine allo-SCT, without impacting GVL responses (Sykes et al. (1990) Proc. Natl. Acad. Sci. U.S.A. 87:5633-5647; Sykes et al. (1990) J. Exp. Med. 171:645-658). In murine allo-HSCT models, co-infusion of Treg expanded ex-vivo with IL-2 also resulted in suppression of GVHD, with improved immune reconstitution and preserved GVL responses (Taylor et al. (2002) Blood 99:3493-3499; Trenado et al. (2003) J. Clin. Invest. 112:1688-1696). Tregs are also important in suppressing inflammation as well. In the context of ongoing inflammation, it is critical that treatments preferentially enhance Tregs without activating conventional T cells (Tcons) or other effectors that may worsen GVHD. Effective augmentation of Tregs in vivo is also directly relevant to other disorders of impaired peripheral tolerance (e.g., autoimmune diseases like SLE, T1D, MS, psoriasis, RA, IBD, vasculitis), where Treg dysfunction is increasingly implicated (Grinberg-Bleyer et al. (2010) J. Exp. Med. 207:1871-1878; Buckner (2010) Nat. Rev. Immunol. 10:849-859; Humrich et al. (2010) Proc. Natl. Acad. Sci. U.S.A. 107:204-209; Carbone et al. (2014) Nat. Med. 20:69-74).

Thus, decreasing the number of Tregs, decreasing Treg activity, and/or increasing Treg cell death (e.g., apoptosis) is generally useful for increasing immune reactions associated with a range of immune disorders (e.g., cancer, infection, and the like). The inverse is also applicable for decreasing immune reactions by upregulating Tregs. For example, effective augmentation of Tregs in vivo is also directly relevant to other disorders of impaired peripheral tolerance (e.g., autoimmune diseases like SLE, T1D, MS, psoriasis, RA, IBD, vasculitis), where Treg dysfunction is increasingly implicated (Grinberg-Bleyer et al. (2010) J. Exp. Med. 207:1871-1878; Buckner (2010) Nat. Rev. Immunol. 10:849-859; Humrich et al. (2010) Proc. Natl. Acad. Sci. U.S.A. 107:204-209; Carbone et al. (2014) Nat. Med. 20:69-74).

Modulation of Treg activity, Teff activity, and Treg:Teff interactions can be determined according to well-known methods in the art and as exemplified in the Examples. For example, Tregs and/or Teffs proliferation, activity, apoptosis, cytokine production repertoire, Tregs activity, Tregs apoptosis, CD25 expression, phosphorylated STATS (pSTAT5) expression, FOXP3 expression, and the like can be analyzed. Moreover, phenotypic analyses of lymphocyte subsets, functional assays of immunomodulation leading to reduced immune responses, plasma cytokines, and the like can be analyzed as described further herein.

Such well-known immune cell characteristics can also be used to purify, enrich, and/or isolate Tregs, or alternatively, reduce or determine reduction of Tregs. For example, the term “enriched Tregs” refer to a composition comprising Tregs in addition to other T cells in a proportion where the composition has at least a 1:2, 1:1.9, 1:1.8, 1:1.7, 1:1.6, 1:1.5, 1:1.4, 1:1.3, 1:1.2, 1:1.1, 1:1, 1:0.9, 1:0.8, 1:0.7, 1:0.6, 1:0.5, 1:0.4, 1:0.3, 1:0.2, 1:0.1, or more, or any range in between or any value in between, ratio of Tregs to Tcons/Teffs, to CD3+ cells, or to other benchmark. Such ratios can be achieved by purifying a composition comprising T cells with various methodologies, such as CD8+ and CD19+ co-depletion in combination with positive selection for CD25+ cells. Such enriched Tregs can further be defined in terms of cell markers and/or viability. For example, an enriched Tregs cell composition can have greater than 40%, 45%, 50%, 55%, 60%, 65%, 70%, 75%, 80%, 85%, 90%, 95%, 99%, or more, or any range in between or any value in between, total cell viability. It can comprise greater than 40%, 45%, 50%, 55%, 60%, 65%, 70%, 75%, 80%, 85%, 90%, 95%, 99%, or more, or any range in between or any value in between, CD4+CD25+ cells. It can comprise greater than 40%, 45%, 50%, 55%, 60%, 65%, 70%, 75%, 80%, 85%, 90%, 95%, 99%, or more, or any range in between or any value in between, FoxP3+ cells. Similarly, the term “reduced Tregs” refers to a reduction in Tregs and can be quantified and qualified according to the inverse of the description provided above for enriched Tregs.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluid that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, and vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. In some embodiments, such cells exhibit such characteristics in part or in full due to the expression and activity of oncogenes or the defective expression and/or activity of tumor suppressor genes, such as retinoblastoma protein (Rb). Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

In certain embodiments, the cancer is breast cancer. Breast cancer is cancer that develops from breast tissue. Breast cancer may be induced by many factors. In less than 5% of cases, genetics plays a more significant role by causing a hereditary breast-ovarian cancer syndrome (Boris Pasche (2010) Cancer Genetics (Cancer Treatment and Research). Berlin: Springer. pp. 19-20). This includes those who carry the BRCA1 and BRCA2 gene mutation. These mutations account for up to 90% of the total genetic influence with a risk of breast cancer of 60-80% in those affected. Other significant mutations include p53 (Li-Fraumeni syndrome), PTEN (Cowden syndrome), and STK11 (Peutz-Jeghers syndrome), CHEK2, ATM, BRIP1, and PALB2 (Gage et al. (2012) Journal of surgical oncology 105:444-451).

In some embodiments, the cancer expresses retinoblastoma protein (Rb). The crucial role for the RB pathway in breast cancer therapeutic response or prognosis is already well-known. For reviews and reports, see, at least, Witkiewicz and Knudsen (2014) Breast Cancer Res. 16:207; Ertel et al. (2010) Cell Cycle 9:4153-4163; and Scambia et al. (2006) Oncogene 25:5302-5308. In other embodiments, the cancer has decreased expression of and/or defective Rb (e.g., due to genetic mutations, deletions, modifications, or other defects).

In some embodiments, the cancer is “estrogen positive breast cancer” or “(ER+) breast cancer,” which refers to breast cancers that are estrogen receptor (ER) positive. Breast cancer is the most common cancer affecting women and accounts for 26% of newly diagnosed cancers (Cecchini et al. (2015) Cureus 7(10):e364). Of these cancers, over 80% will express either the estrogen or progesterone receptor and be amenable to hormonal therapy (Howlader et al. (2014) J Natl Cancer Inst. 106). The use of aromatase inhibitors, anti-estrogens, tamoxifen, or fulvestrant is associated with a significant reduction in breast cancer recurrence and improved overall survival (Davies et al. (2011) Lancet 378:771-784). However, most patients with advanced disease eventually develop resistance to these therapies. Breast-conserving surgery has been shown to have equivalent outcomes to mastectomy when combined with radiation therapy and has become the main treatment method for breast cancer patients (Clarke et al. (2005) Lancet 366:2087-2106). Thereby, there are a substantial number of women who receive radiation and hormonal therapy.

Estradiol activates proliferation through transcriptional activation of c-Myc and cyclin D, which allow for downstream activation of the cyclin-dependent kinases required for progression from G1 into S phase of the cell cycle (Schmidberger et al. (2003) Endocr Relat Cancer 10:375-388). This activity of estrogen is required for the proliferation of the cancer cells; tamoxifen or aromatase inhibitors are utilized to block this pathway (Schmidberger et al. (2003) Endocr Relat Cancer 10:375-388). Treatment of cells with tamoxifen or aromatase inhibitors results in an accumulation of cells in the G1 phase of the cell cycle. Radiation sensitivity depends on the stage of the cell cycle, with cells in G2/M being the most sensitive to radiation changes (Sinclair et al. (1966) Radiat Res. 29:450-474). Therefore, it is possible that hormonal therapy may reduce the efficacy of radiation by arresting the cells in a stage of the cell cycle that is more resistant to DNA damage.

As used herein, “endocrine therapies” are first-line treatments for estrogen receptor-positive (ER+) breast cancer, such as selective ER modulation using tamoxifen or anti-estrogens, aromatase inhibitors, nonsteroidal drugs (e.g., letrozol, anastrozol, and vostrozol), steroidal drugs (e.g., exemestane), ovarian ablation surgery, ovarian ablation radiotherapy, LHRH analog therapy, anti-HER-2 antibodies, anti-ER antibodies, anti-PR antibodies, and the like. Representative endocrine therapies are further described below (see US2007/0192880). Although complementation and convergence of various signaling pathways are ultimately responsible for the physiology and pathophysiology of breast tissue, it is clear that estrogens are primary agents in the development of most breast cancers by stimulating and maintaining malignant cell proliferation. Consequently, measures that perturb the estrogen environment of the tumor cells by blocking the synthesis of estrogen or by preventing estrogen actions are current strategies for therapeutic intervention for the neoplasm. The management of early breast cancer is primarily based on surgical removal of the tumor by mastectomy or lumpectomy without or with radiotherapy, followed by an adjuvant systemic therapy dependent upon the ER status.

The term “coding region” refers to regions of a nucleotide sequence comprising codons which are translated into amino acid residues, whereas the term “non-coding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid, or protein is the activity/level of expression or copy number in a biological sample, e.g., a sample containing tissue, whole blood, serum, plasma, buccal scrape, saliva, cerebrospinal fluid, urine, stool, and bone marrow, from a subject, e.g., a human, not afflicted with cancer, or from a corresponding non-cancerous tissue in the same subject who has cancer.

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present invention. One example is determining whether to provide targeted therapy against a cancer to provide anti-cancer therapy (e.g., CDK4/6 inhibitor therapy). Another example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “expression signature” or “signature” refers to a group of two or more coordinately expressed biomarkers. For example, the genes, proteins, and the like making up this signature may be expressed in a specific cell lineage, stage of differentiation, or during a particular biological response. The biomarkers can reflect biological aspects of the tumors in which they are expressed, such as the cell of origin of the cancer, the nature of the non-malignant cells in the biopsy, and the oncogenic mechanisms responsible for the cancer. Expression data and gene expression levels can be stored on computer readable media, e.g., the computer readable medium used in conjunction with a microarray or chip reading device. Such expression data can be manipulated to generate expression signatures.

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

The term “homologous” refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer. As described above, immunotherapy against immune checkpoint targets, such as PD-1, PD-L1, PD-L2, CTLA-4, and the like are useful.

The term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

“Anti-immune checkpoint” or “immune checkpoint inhibitor or “immune checkpoint blockade” therapy refers to the use of agents that inhibit immune checkpoint nucleic acids and/or proteins. Immune checkpoints share the common function of providing inhibitory signals that suppress immune response and inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can bind to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy). Numerous immune checkpoint inhibitors are known and publicly available including, for example, Keytruda® (pembrolizumab; anti-PD-1 antibody), Opdivo® (nivolumab; anti-PD-1 antibody), Tecentriq® (atezolizumab; anti-PD-L1 antibody), durvalumab (anti-PD-L1 antibody), and the like.

The term “immune disorders” refers to conditions characterized by an unwanted immune response. In some embodiments, the immune disorder is such that a desired anti-immune disorder response suppresses immune responses. Such conditions in which downregulation of an immune response is desired are well-known in the art and include, without limitation, situations of tissue, skin and organ transplantation, in graft-versus-host disease (GVHD), inflammation, or in autoimmune diseases, such as systemic lupus erythematosus, multiple sclerosis, allergy, hypersensitivity response, and a disorder requiring increased regulatory T cell production or function, as described further herein. In other embodiments, the immune disorder is such that a desired response is an increased immune response. Such conditions in which upregulation of an immune response is desired are well-known in the art and include, without limitation, disorders requiring increased CD4+ effector T cell production or function such as combating cancer, infections (e.g., parasitic, bacterial, helminthic, or viral infections), a disorder requiring improved vaccination efficiency, and the like).

The term “immune response” includes T cell mediated and/or B cell mediated immune responses. Exemplary immune responses include T cell responses, e.g., cytokine production and cellular cytotoxicity. In addition, the term immune response includes immune responses that are indirectly affected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” or “deficient” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented. Similarly, a biological function, such as the function of a protein, is inhibited if it is decreased as compared to a reference state, such as a control like a wild-type state. For example, CDK activity of a CDK4 or CDK6 protein that is contacted with a CDK4 or CDK6 inhibitor is inhibited or deficient if the stability of CDK4 or CDK6 kinase is decreased due to contact with the CDK4 or CDK6 inhibitor, in comparison to the CDK4 or CDK6 protein not contacted with the CDK4 or CDK6 inhibitor. Similarly, kinase activity of a mutant CDK4 or CDK6 kinase is inhibited or deficient if the kinase activity is decreased due to the mutation and/or contact with the inhibitor, in comparison to the wild-type CDK4 or CDK6 kinase and/or the mutant CDK4 or CDK6 kinase not contacted with the inhibitor. Such inhibition or deficiency can be induced, such as by application of agent at a particular time and/or place, or can be constitutive, such as by a heritable mutation. Such inhibition or deficiency can also be partial or complete (e.g., essentially no measurable activity in comparison to a reference state, such as a control like a wild-type state). Essentially complete inhibition or deficiency is referred to as blocked.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

A “kit” is any manufacture (e.g. a package or container) comprising at least one reagent, e.g. a probe or small molecule, for specifically detecting and/or affecting the expression of a marker of the invention. The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

The term “neoadjuvant therapy” refers to a treatment given before the primary treatment. Examples of neoadjuvant therapy can include chemotherapy, radiation therapy, and hormone therapy.

The “normal” level of expression of a biomarker is the level of expression of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

Such “significance” levels can also be applied to any other measured parameter described herein, such as for expression, inhibition, cytotoxicity, cell growth, and the like.

The term “pre-determined” biomarker amount and/or activity measurement(s) may be a biomarker amount and/or activity measurement(s) used to, by way of example only, evaluate a subject that may be selected for a particular treatment, evaluate a response to a treatment such as one or more CDK4 or CDK6 inhibitors alone or in combination with one or more CDK4 or CDK6 inhibitors, and/or evaluate the disease state. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cancer to anti-cancer therapy, such as CDK4 or CDK6 inhibitor therapy (e.g., CDK4 or CDK6 inhibitors either alone or in combination with an immunotherapy, such as an immune checkpoint inhibition therapy). Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC) and/or biomarker target, or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to a particular anti-cancer therapy (e.g., CDK4 and/or CDK6 inhibitors either alone or in combination with an immunotherapy) or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as lung cancer, melanoma, and renal cell carcinoma), development of one or more clinical factors, development of intestinal cancer, or recovery from the disease.

The term “response to therapy (e.g., CDK4 or CDK6 inhibitors either alone or in combination with an immunotherapy, such as an immune checkpoint inhibition therapy)” relates to any response to therapy (e.g., CDK4 or CDK6 inhibitors either alone or in combination with an immunotherapy, such as an immune checkpoint inhibition therapy), and, for cancer, preferably to a change in cancer cell numbers, tumor mass, and/or volume after initiation of neoadjuvant or adjuvant chemotherapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for whom biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 5% or more, for example, 10%, 15%, 20%, 25%, 30%, 35%, 40%, 45%, 50%, 55%, 60%, 65%, 70%, 75%, 80%, 85%, 90%, 95%, 100%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal who is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to response to therapey. For example, an anti-cancer response includes reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs, shRNAs, or other RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

In addition to RNAi, genome editing can be used to modulate the copy number or genetic sequence of a biomarker of interest, such as constitutive or induced knockout or mutation of a CDK4 and/or CDK6 biomarker of interest. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

The term “selective inhibition” or “selectively inhibit” as applied to a biologically active agent refers to the agent's ability to selectively reduce the target signaling activity as compared to off-target signaling activity, via direct or interact interaction with the target. For example, an agent that selectively inhibits CDK4 and/or CDK6 over another CDK kinase may have an activity against CDK4 and/or CDK6 that is at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 2× (times) more than the compound's activity against at least one of other CDKs (e.g., at least about 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, 105×, 110×, 120×, 125×, 150×, 200×, 250×, 300×, 350×, 400×, 450×, 500×, 600×, 700×, 800×, 900×, 1000×, 1500×, 2000×, 2500×, 3000×, 3500×, 4000×, 4500×, 5000×, 5500×, 6000×, 6500×, 7000×, 7500×, 8000×, 8500×, 9000×, 9500×, 10000×, or greater, or any range in between, inclusive). For comparison, the other CDKs described herein may be at least one of CDK1, CDK2, CDK3, CDK5, CDK7, CDK8, CDK9, CDK10, CDK11, CDK12, CDK13, or other non-CDK4/6 CDKs. Such metrics are typically expressed in terms of relative amounts of agent required to reduce activity by half.

More generally, the term “selective” refers to a preferential action or function. The term “selective” can be quantified in terms of the preferential effect in a particular target of interest relative to other targets. For example, a measured variable (e.g., reduction of Tregs versus other cells, such as other immune cells) can be 10%, 20%, 25%, 30%, 35%, 40%, 45%, 50%, 55%, 60%, 65%, 70%, 75%, 80%, 85%, 90%, 95%, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 70-fold, 80-fold, 90-fold, 100-fold, or greater or any range in between inclusive (e.g., 50% to 16-fold), different in a target of interest versus unintended or undesired targets. The same fold analysis can be used to confirm the magnitude of an effect in a given tissue, cell population, measured variable, measured effect, and the like, such as the Tregs:Teffs ratio, hyperproliferative cell growth rate or volume, Tregs proliferation rate, and the like.

By contrast, the term “specific” refers to an exclusionary action or function. For example, specific modulation of CDK4 and/or CDK6 refers to the exclusive modulation of CDK4 and/or CDK6 and not in other CDK family members. In another example, specific binding of an antibody to a predetermined antigen refers to the ability of the antibody to bind to the antigen of interest without binding to other antigens. Typically, the antibody binds with an affinity (K_{D}) of approximately less than 1×10⁻⁷ M, such as approximately less than 10⁻⁸M, 10⁻⁹M, 10⁻¹⁰ M, 10⁻¹¹M, or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. In addition, K_{D} is the inverse of K_{A}. The phrases “an antibody recognizing an antigen” and “an antibody specific for an antigen” are used interchangeably herein with the term “an antibody which binds specifically to an antigen.”

The term “sensitize” means to alter cells, such as cancer cells or tumor cells, in a way that allows for more effective treatment with a therapy (e.g., CDK4 and/or CDK6 inhibitors either alone or in combination with an immunotherapy, such as an immune checkpoint inhibition therapy). In some embodiments, normal cells are not affected to an extent that causes the normal cells to be unduly injured by the therapy (e.g., CDK4 and/or CDK6 inhibitors either alone or in combination with an immunotherapy, such as an immune checkpoint inhibition therapy). An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 months for human and 4-6 weeks for mouse. A composition or a method sensitizes response to a therapeutic treatment if the increase in treatment sensitivity or the reduction in resistance is 5% or more, for example, 10%, 15%, 20%, 25%, 30%, 35%, 40%, 45%, 50%, 55%, 60%, 65%, 70%, 75%, 80%, 85%, 90%, 95%, 100%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “synergistic effect” refers to the combined effect of two or more therapeutic agents, such as two or more CDK4 and/or CDK6 inhibitors, a CDK4 and/or CDK6 inhibitor and an immunotherapy, CDK4 and/or CDK6 inhibitors either alone or in combination with an immunotherapy, such as an immune checkpoint inhibition therapy, and the like, can be greater than the sum of the separate effects of the anticancer agents alone.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a patient having or at risk for having cancer, to inhibit expression of a biomarker gene which is overexpressed in cancer and thereby treat, prevent, or inhibit cancer in the subject.

The term “subject” refers to any healthy animal, mammal or human, or any animal, mammal or human afflicted with a cancer, e.g., lung, ovarian, pancreatic, liver, breast, prostate, and colon carcinomas, as well as melanoma and multiple myeloma. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. Cancer cell death can be promoted by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in cancer cell numbers and/or a solid malignancy can be achieved.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

### Genetic Code

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention and related biomarkers (e.g., biomarkers listed in Table 1) are well-known in the art and readily available on publicly available databases, such as the National Center for Biotechnology Information (NCBI). For example, exemplary nucleic acid and amino acid sequences derived from publicly available sequence databases are provided below.

Representative sequences of the biomarkers described above are presented below in Table 1. It is to be noted that the terms described above can further be used to refer to any combination of features described herein regarding the biomarkers. For example, any combination of sequence composition, percentage identify, sequence length, domain structure, functional activity, etc. can be used to describe a biomarker of the present invention.

TABLE 1
SEQ ID NO: 1 Human CDK4 cDNA sequence (NM_000075.3) (CDS from positions
293 to 1204)

| | |
|---|---|
| 1 | cacctcctgt ccgcccctca gcgcatgggt ggcggtcacg tgcccagaac gtccggcgtt |
| 61 | cgccccgccc tcccagtttc cgcgcgcctc tttggcagct ggtcacatgg tgagggtggg |
| 121 | ggtgaggggg cctctctagc ttgcggcctg tgtctatggt cgggccctct gcgtccagct |
| 181 | gctccggacc gagctcgggt gtatggggcc gtaggaaccg gctccggggc cccgataacg |
| 241 | ggccgccccc acagcacccc gggctggcgt gagggtctcc cttgatctga gaatggctac |
| 301 | ctctcgatat gagccagtgg ctgaaattgg tgtcggtgcc tatgggacag tgtacaaggc |
| 361 | ccgtgatccc cacagtggcc actttgtggc cctcaagagt gtgagagtcc ccaatggagg |
| 421 | aggaggtgga ggaggccttc ccatcagcac agttcgtgag gtggctttac tgaggcgact |
| 481 | ggaggctttt gagcatccca atgttgtccg gctgatggac gtctgtgcca catcccgaac |
| 541 | tgaccgggag atcaaggtaa ccctggtgtt tgagcatgta gaccaggacc taaggacata |
| 601 | tctggacaag gcacccccac caggcttgcc agccgaaacg atcaaggatc tgatgcgcca |
| 661 | gtttctaaga ggcctagatt tccttcatgc caattgcatc gttcaccgag atctgaagcc |
| 721 | agagaacatt ctggtgacaa gtggtggaac agtcaagctg gctgactttg gcctggccag |
| 781 | aatctacagc taccagatgg cacttacacc cgtggttgtt acactctggt accgagctcc |
| 841 | cgaagttctt ctgcagtcca catatgcaac acctgtggac atgtggagtg ttggctgtat |
| 901 | ctttgcagag atgtttcgtc gaaagcctct cttctgtgga aactctgaag ccgaccagtt |
| 961 | gggcaaaatc tttgacctga ttgggctgcc tccagaggat gactggcctc gagatgtatc |
| 1021 | cctgccccgt ggagcctttc cccccagagg gccccgccca gtgcagtcgg tggtacctga |
| 1081 | gatggaggag tcgggagcac agctgctgct ggaaatgctg acttttaacc cacacaagcg |
| 1141 | aatctctgcc tttcgagctc tgcagcactc ttatctacat aaggatgaag gtaatccgga |
| 1201 | gtgagcaatg gagtggctgc catggaagga agaaaagctg ccatttccct tctggacact |
| 1261 | gagagggcaa tctttgcctt tatctctgag gctatggagg gtcctcctcc atctttctac |
| 1321 | agagattact ttgctgcctt aatgacattc ccctcccacc tctccttttg aggcttctcc |
| 1381 | ttctccttcc catttctcta cactaagggg tatgttccct cttgtccctt tccctacctt |
| 1441 | tatatttggg gtcctttttt atacaggaaa aacaaaacaa agaaataatg gtcttttttt |
| 1501 | tttttttaat gtttcttcct ctgtttggct ttgccattgt gcgatttgga aaaaccactt |
| 1561 | ggaagaaggg actttcctgc aaaaccttaa agactggtta aattacaggg cctaggaagt |
| 1621 | cagtggagcc ccttgactga caaagcttag aaaggaactg aaattgcttc tttgaatatg |
| 1681 | gattttaggc ggggcgtggt ggctcacgcc tataatccca gcacgttggg aggccaacgc |
| 1741 | gggtggatca cctgaggtca ggagttcgag accagcctga ctaacatggt gaaaccctgt |
| 1801 | ctctactaaa aatacaaaat tagtcaggcg tggtggtgca cacctgtaat cccagctact |
| 1861 | tgggagactg aggcaggagg atcgcttgaa cccgggaggc agaggttgcg gtgagccgag |
| 1921 | atcatgccat tgcactccag cctgggcaac agagcaagac tctgtgtcaa aaaaaaaaaa |
| 1981 | agaatataga tttttaaatg gcaaaaaaaa aaaaaaaaaa |

SEQ ID NO: 2 Human CDK4 amino acid sequence (NP_000066.1)

| | |
|---|---|
| 1 | matsryepva eigvgaygtv ykardphsgh fvalksvrvp nggggggglp istvrevall |
| 61 | rrleafehpn vvrlmdvcat srtdreikvt lvfehvdqdl rtyldkappp glpaetikdl |
| 121 | mrqflrgldf lhancivhrd lkpenilvts ggtvkladfg lariysyqma ltpvvvtlwy |
| 181 | rapevllqst yatpvdmwsv gcifaemfrr kplfcgnsea dqlgkifdli glppeddwpr |
| 241 | dvslprgafp prgprpvqsv vpemeesgaq lllemltfnp hkrisafral qhsylhkdeg |
| 301 | npe |

SEQ ID NO: 3 Mouse CKD4 cDNA sequence (NM_009870.3) (CDS from positions
174-1085)

| | |
|---|---|
| 1 | gtgggggtga gggggcctct ctagctcgcg gcctgtgtct atggtctggc ccgaagcgtc |
| 61 | cagctgcccg ggaccgatcc ccggtgtatg gcgccgcagg aaccggctcc cgggcccaga |
| 121 | taaagggcca cctccagagc tcttagccga gcgtaagatc ccctgcttcg agaatggctg |
| 181 | ccactcgata tgaacccgtg gctgaaattg gtgtcggtgc ctatgggacg gtgtacaaag |
| 241 | cccgagatcc ccacagtggc cactttgtgg ccctcaagag tgtgagagtt cctaatggag |
| 301 | gagcagctgg agggggcctt cccgtcagca cagttcgtga ggtggccttg ttaaggaggc |
| 361 | tggaggcctt tgaacatccc aatgttgtac ggctgatgga tgtctgtgct acttcccgaa |
| 421 | ctgatcggga catcaaggtc accctagtgt ttgagcatat agaccaggac ctgaggacat |
| 481 | acctggacaa agcacctcca ccgggcctgc cggttgagac cattaaggat ctaatgcgtc |
| 541 | agtttctaag cggcctggat tttcttcatg caaactgcat tgttcaccgg gacctgaagc |
| 601 | cagagaacat tctagtgaca agtaatggga ccgtcaagct ggctgacttt ggcctagcta |
| 661 | gaatctacag ctaccagatg gccctcacgc ctgtggtggt tacgctctgg taccgagctc |
| 721 | ctgaagttct tctgcagtct acatacgcaa cacccgtgga catgtggagc gttggctgta |
| 781 | tctttgcaga gatgttccgt cggaagcctc tcttctgtgg aaactctgaa gccgaccagt |
| 841 | tggggaaaat ctttgatctc attggattgc ctccagaaga cgactggcct cgagaggtat |
| 901 | ctctacctcg aggagccttt gcccccagag ggcctcggcc agtgcagtca gtggtgccag |
| 961 | agatggagga gtctggagcg cagctgctac tggaaatgct gacctttaac ccacataagc |
| 1021 | gaatctctgc cttccgagcc ctgcagcact cctacctgca caaggaggaa agcgacgcag |
| 1081 | agtgagaaga ggggctgcct ttcccagtct tggtggagaa accctcgctg aagcggcagc |
| 1141 | ctctgtttcc ccccaaggct gtggagaatc ctccagtttt ttacagagaa tattttaagc |
| 1201 | cttaaataac aagtccccac ctctccttac gaggttcacc cccattaccc tcccctagct |
| 1261 | ctacactaaa gggcaggtgt atctgtcttc ttccctccct gatttatact gggatctttt |
| 1321 | ttatacagga aaacaagaca agacaaaaaa aaaaaaaaaa aaaaa |

SEQ ID NO: 4 Mouse CDK4 amino acid sequence (NP_034000.1)

| | |
|---|---|
| 1 | maatryepva eigvgaygtv ykardphsgh fvalksvrvp nggaaggglp vstvrevall |
| 61 | rrleafehpn vvrlmdvcat srtdrdikvt lvfehidqd1 rtyldkappp glpvetikdl |
| 121 | mrqflsgldf lhancivhrd lkpenilvts ngtvkladfg lariysyqma ltpvvvtlwy |
| 181 | rapevllqst yatpvdmwsv gcifaemfrr kplfcgnsea dqlgkifdli glppeddwpr |
| 241 | evslprgafa prgprpvqsv vpemeesgaq lllemltfnp hkrisafral qhsylhkees |
| 301 | dae |

SEQ ID NO: 5 Human CDK6 cDNA sequence (transcript variant 1) (NM_001259.6)
(CDS from positions 413-1393)

| | |
|---|---|
| 1 | aacctctccg cgcgaagacg gcttcagccc tgcagggaaa gaaaagtgca atgattctgg |
| 61 | actgagacgc gcttgggcag aggctatgta atcgtgtctg tgttgaggac ttcgcttcga |
| 121 | ggagggaaga ggagggatcg gctcgctcct ccggcggcgg cggcggcggc gactctgcag |
| 181 | gcggagtttc gcggcggcgg caccagggtt acgccagccc cgcggggagg tctctccatc |
| 241 | cagcttctgc agcggcgaaa gccccagcgc ccgagcgcct gagccggcgg ggagcaagta |
| 301 | aagctagacc gatctccggg gagccccgga gtaggcgagc ggcggccgcc agctagttga |
| 361 | gcgcaccccc cgcccgcccc agcggcgccg cggcgggcgg cgtccaggcg gcatggagaa |
| 421 | ggacggcctg tgccgcgctg accagcagta cgaatgcgtg gcggagatcg gggagggcgc |
| 481 | ctatgggaag gtgttcaagg cccgcgactt gaagaacgga ggccgtttcg tggcgttgaa |
| 541 | gcgcgtgcgg gtgcagaccg gcgaggaggg catgccgctc tccaccatcc gcgaggtggc |
| 601 | ggtgctgagg cacctggaga ccttcgagca ccccaacgtg gtcaggttgt ttgatgtgtg |
| 661 | cacagtgtca cgaacagaca gagaaaccaa actaacttta gtgtttgaac atgtcgatca |
| 721 | agacttgacc acttacttgg ataaagttcc agagcctgga gtgcccactg aaaccataaa |
| 781 | ggatatgatg tttcagcttc tccgaggtct ggactttctt cattcacacc gagtagtgca |
| 841 | tcgcgatcta aaaccacaga acattctggt gaccagcagc ggacaaataa aactcgctga |
| 901 | cttcggcctt gcccgcatct atagtttcca gatggctcta acctcagtgg tcgtcacgct |
| 961 | gtggtacaga gcacccgaag tcttgctcca gtccagctac gccacccccg tggatctctg |
| 1021 | gagtgttggc tgcatatttg cagaaatgtt tcgtagaaag cctctttttc gtggaagttc |
| 1081 | agatgttgat caactaggaa aaatcttgga cgtgattgga ctcccaggag aagaagactg |
| 1141 | gcctagagat gttgcccttc ccaggcaggc ttttcattca aaatctgccc aaccaattga |
| 1201 | gaagtttgta acagatatcg atgaactagg caaagaccta cttctgaagt gtttgacatt |
| 1261 | taacccagcc aaaagaatat ctgcctacag tgccctgtct cacccatact tccaggacct |
| 1321 | ggaaaggtgc aaagaaaacc tggattccca cctgccgccc agccagaaca cctcggagct |
| 1381 | gaatacagcc tgaggcctca gcagccgcct taagctgatc ctgcggagaa cacccttggt |
| 1441 | ggcttatggg tccccctcag caagccctac agagctgtgg aggattgcta tctggaggcc |
| 1501 | ttccagctgc tgtcttctgg acaggctctg cttctccaag gaaaccgcct agtttactgt |
| 1561 | tttgaaatca atgcaagagt gattgcagct ttatgttcat ttgtttgttt gtttgtctgt |
| 1621 | ttgtttcaag aacctggaaa aattccagaa gaagagaagc tgctgaccaa ttgtgctgcc |
| 1681 | atttgatttt tctaaccttg aatgctgcca gtgtggagtg ggtaatccag gcacagctga |
| 1741 | gttatgatgt aatctctctg cagctgccgg gcctgatttg gtacttttga gtgtgtgtgt |
| 1801 | gcatgtgtgt gtgtgtgtgt gtgtgtgtgt gtgtgtatgt gagagattct gtgatctttt |
| 1861 | aaagtgttac tttttgtaaa cgacaagaat aattcaattt taaagactca aggtggtcag |
| 1921 | taaataacag gcatttgttc actgaaggtg attcaccaaa atagtcttct caaattagaa |
| 1981 | agttaacccc atgtcctcag catttctttt ctggccaaaa gcagtaaatt tgctagcagt |
| 2041 | aaaagatgaa gttttataca cacagcaaaa aggagaaaaa attctagtat attttaagag |
| 2101 | atgtgcatgc attctattta gtcttcagaa tgctgaattt acttgttgta agtctatttt |
| 2161 | aaccttctgt atgacatcat gctttatcat ttcttttgga aaatagcctg taagcttttt |
| 2221 | attacttgct ataggtttag ggagtgtacc tcagatagat tttaaaaaaa agaatagaaa |
| 2281 | gcctttattt cctggtttga aattcctttc ttcccttttt ttgttgttgt tattgttgtt |
| 2341 | tgttgttgtt attttgtttt tgtttttagg aatttgtcag aaactctttc ctgttttggt |
| 2401 | ttggagagta gttctctcta actagagaca ggagtggcct tgaaattttc ctcatctatt |
| 2461 | acactgtact ttctgccaca cactgccttg ttggcaaagt atccatcttg tctatctccc |
| 2521 | ggcacttctg aaatatattg ctaccattgt ataactaata acagattgct taagctgttc |
| 2581 | ccatgcacca cctgtttgct tgctttcaat gaacctttca taaattcgca gtctcagctt |
| 2641 | atggtttatg gcctcgattc tgcaaaccta acagggtcac atatgttctc taatgcagtc |
| 2701 | cttctacctg gtgtttactt ttgttaccta aataatgagt aggatcttgt tttgttttat |
| 2761 | caccagcaca cagattgcta taaactgtta ctttgtgaat tacattttta tagaagatat |
| 2821 | tttcagtgtc tttacctgag ggtatgtctt tagctatgtt ttagggccat acatttactc |
| 2881 | tatcaaatga tcttttctcc atcccccagg ctgtgcttat ttctagtgcc ttgtgctcac |
| 2941 | tcctgctctc tacagagcca gcctggcctg ggcattgtaa acagcttttc ctttttctct |
| 3001 | tactgttttc tctacagtcc tttatatttc ataccatctc tgccttataa gtggtttagt |
| 3061 | gctcagttgg ctctagtaac cagaggacac agaaagtatc ttttggaaag tttagccacc |
| 3121 | tgtgctttct gactcagagt gcatgcaaca gttagatcat gcaacagtta gattatgttt |
| 3181 | agggttagga ttttcaaaga atggaggttg ctgcactcag aaaataattc agatcatgtt |
| 3241 | tatgcattat taagttgtac tgaattcttt gcagcttaat gtgatatatg actatcttga |
| 3301 | acaagagaaa aaactaggag atgtttctcc tgaagagctt ttggggttgg gaactattct |
| 3361 | tttttaattg ctgtactact taacattgtt ctaattcagt agcttgagga acaggaacat |
| 3421 | tgttttctag agcaagataa taaaggagat gggccataca aatgttttct actttcgttg |
| 3481 | tgacaacatt gattaggtgt tgtcagtact ataaatgctt gagatataat gaatccacag |
| 3541 | cattcaaggt caggtctact caaagtctca catggaaaag tgagttctgc ctttcctttg |
| 3601 | atcgagggtc aaaatacaaa gacatttttg ctagggccta caaattgaat ttaaaaactc |
| 3661 | actgcactga ttcatctgag ctttttggtt agtattcatg gctagagtga acatagcttt |
| 3721 | agtttttgct gttgtaaaag tgttttcata agttcactca agaaaaatgc agctgttctg |
| 3781 | aactggaatt tttcagcatt ctttagaatt ttaaatgagt agagagctca acttttattc |
| 3841 | ctagcatctg cttttgactc atttctaggc agtgcttatg aagaaaaatt aaagcacaaa |
| 3901 | cattctggca ttcaatcgtt ggcagattat cttctgatga cacagaatga aagggcatct |
| 3961 | cagcctctct gaactttgta aaaatctgtc cccagttctt ccatcggtgt agttgttgca |
| 4021 | tttgagtgaa tactctcttg atttatgtat tttatgtcca gattcgccat ttctgaaatc |
| 4081 | cagatccaac acaagcagtc ttgccgttag ggcattttga agcagatagt agagtaagaa |
| 4141 | cttagtgact acagcttatt cttctgtaac atatggtttc aaacatcttt gccaaaagct |
| 4201 | aagcagtggt gaactgaaaa gggcatattg ccccaaggtt acactgaagc agctcatagc |
| 4261 | aagttaaaat attgtgacag atttgaaatc atgtttgaat ttcatagtag gaccagtaca |
| 4321 | agaatgtccc tgctagtttc tgtttgatgt ttggttctgg cggctcaggc attttgggaa |
| 4381 | ctgttgcaca gggtggagtc aaaacaacct acatataaaa agagaaaaag agaaacttgt |
| 4441 | ccatttagct ttcataagaa atcccatggc aaagggtaat aaaaaggacc taatcttaaa |
| 4501 | aatacaattt ctaagcactt gtaagaaccc agtgggttgg agcctcccac tttgtccctc |
| 4561 | ctttgaagtg gatgggaact caaggtgcaa agaacctgtt ttggaagaaa gcttggggcc |
| 4621 | atttcagccc cctgtattct catgattttc tctcaggaag cacacactgt gaatggcaga |
| 4681 | cttttcattt agccccaggt gacttactaa aaatagttga aaattattca cctaagaata |
| 4741 | gaatctcagc attgtgttaa ataaaaatga aagctttaga aggcatgaga tgttcctatc |
| 4801 | ttaaataaag catgtttctt ttctatagag aaatgtatag tttgactctc cagaatgtac |
| 4861 | tatccatctt gatgagaaaa ctcttaaata gtaccaaaca ttttgaactt taaattatgt |
| 4921 | atttaaagtg agtgtttaag aaactgtagc tgcttctttt acaagtggtg cctattaaag |
| 4981 | tcagtaatgg ccattattgt tccattgtgg aaattaaatt atgtaagctt cctaatatca |
| 5041 | taaacatatt aaaattcttc taaaatattg cttttctttt aagtgacaat ttgactattc |
| 5101 | ttatgataag cacatgagag tgtcttacat tttccaaaag caggctttaa ttgcatagtt |
| 5161 | gagtctagga aaaaataatg ttaaaagtga atatgccacc ataattactt aattatgtta |
| 5221 | gtatagaaac tacagaatat ttaccctgga aagaaaatat tggaatgtta ttataaactc |
| 5281 | ttagatattt atataattca aaagaatgca tgtttcacat tgtgacagat aaagatgtat |
| 5341 | gatttctaag gctttaaaaa ttattcataa aacagtgggc aatagataaa ggaaattctg |
| 5401 | gagaaaatga aggtatttaa agggtagttt caaagctata tatattttga aggatatatt |
| 5461 | ctttatgaac aaatatattg taaaaattta tactaaggtc atctggtaac tgtgggatta |
| 5521 | atatggtcga aaacaaatgt tatggagaag ctgtcccaag caaactaaat tacctgtact |
| 5581 | tttttcccat ttcaagggaa gaggcaacca catgaagcaa tacttcttac acatgcctaa |
| 5641 | gaacgttcat tgaaaaaata aatttttaaa aggcatgtgt ttcctatgcc accaatactt |
| 5701 | ttgaaaaatt gtgaacctta cccaaaacca tttatcatgt ccattaagta tatttgggta |
| 5761 | tataattagg aagatattta catgttccat ctccacagtg gaaaaactta ttgaggctac |
| 5821 | caaagtgtgc caagaaatgt aagtccttag agtaattaga aatgctgttt tcctcaaaag |
| 5881 | catgagaaac tagcattttc atttcttatt tactcccttt ctatatcaat gcaattcaca |
| 5941 | acccaatttt aatacatccc tatatctcaa gcatttctat cttgtacttt ttcagaaaat |
| 6001 | aaaccaaaaa taatcctttg gtctctctat cttctgacct ttgtaagcaa cagaaatgta |
| 6061 | aaaacagaag gggtccaatt tttacacgtt tttttctcaa gtagcctttc tggggatttt |
| 6121 | tattttctta atgaagtgcc aatcagcttt tcaaaatgtt ttctatttct cagcatttcc |
| 6181 | aggaagtgat aacgtttagc taaatgagta gaagtggact tccttcaaca tattgttacc |
| 6241 | ttgtctagcc ttaggaagaa aacaagagcc acctgaaaat aaatacaggc tcttttcgag |
| 6301 | catctgctga aatactgtta cagcaatttg aagttgatgt ggtaggaaag gaaggtgact |
| 6361 | tttcttgcaa aagtctttct aaacattcac actgtcctaa gagatgagct ttcttgtttt |
| 6421 | attccggtat attccacaag gtggcacttt tagagaaaaa caaatctgat gaagactaaa |
| 6481 | gaggtacttc taaaagagat ttcattctaa ctttattttt ctgcgcatat ttaactcttt |
| 6541 | cctagcactt gttttttggg atgattaata gtctctataa tgttctgtaa cttcaatatt |
| 6601 | ttacttgtta cctaggttct gaacaattgt ctgcaaataa attgttctta aggatggata |
| 6661 | atacacccat tttgatcatt taagtaaaga aagcctagtc attcattcag tcaagaaaaa |
| 6721 | atttttgaag tacccagtta ccttactttt ctagattaaa acaggcttag ttactaaaaa |
| 6781 | ggcagtcctc atctgtgaac aggatagttt cgttagaagt ataaaactcc tttagtggcc |
| 6841 | ccagttaaaa cacacatacc ctctctgctg ctttcaaatt ccctagcatg gtggcctttc |
| 6901 | aacattgatt aaattttaaa atcctaattt aaagatcagg tgagcaaaat gagtagcaca |
| 6961 | tcagtaattc agtagacaaa acttttgtct gaaaaattgc tgtattgaaa cagagcccta |
| 7021 | aaataccaaa agaccaggta attttaacat ttgtggaatc acaaatgtaa attcataaga |
| 7081 | agctctaatt aaaaaaaaaa agtctgaagt atatgagcat aacaacttag gagtgtgtct |
| 7141 | acatacttaa cttttgaagt tttttggcaa ctttatatac tttttttaaa tttacaagtc |
| 7201 | tacttaaaga cttcttatac cccaaatgat taagttaatt ttagaggtca cctttctcac |
| 7261 | agcagtgtca cttgaaattt agtagggaag gatattgcag tatttttcag tttccttagc |
| 7321 | acagcaccac agaaagcagc ttattccttt tgagtggcag acactcgacg gtgcctgccc |
| 7381 | aactttcctc ctgagtggca agcagatgag tctcagtaat tcatactgaa ccaaaatgcc |
| 7441 | acatacacta ggggcagtca gaaactggct gagaaatccc ccgcctcatt cgcccctctg |
| 7501 | ctcccaggaa ctagagtcca gttaaagccc ctatgcgaaa ggccgaattc caccccaggg |
| 7561 | tttgttataa cagtggccag tctgaacccc atttgctcgt gctcaaaact tgattcccac |
| 7621 | ttgaaagcct tccgggcgcg ctgcctcgtt gccccgcccc tttggcagga gagaggcagt |
| 7681 | gggcgaggcc gggctggggc cccgcctccc actcacctgc cggtgcctga aattatgtgc |
| 7741 | ggccccgcgg gctgctttcc gaggtcagag tgccctgctg ctgtctcaga ggcatctgtt |
| 7801 | ctgcaaatct taggaagaaa aatgtcccta gtagcaaacg ggtgtcttct gtgcataaat |
| 7861 | aagtacaaca caattctccg aaagttcggg taaaaagaga tgcggtagca gctgccctgt |
| 7921 | gtgaagctgt ctaccccgca tctctcaggc gctaagctca gtttttgttt ttgtttttgt |
| 7981 | ttttttaaag aaaagatgta taattgcagg aatttttttt tattttttta ttttccatca |
| 8041 | ttctatatat gtgatggtga aagatatgcc tggaaaagtt ttgttttgaa aagtttattt |
| 8101 | tctgcttcgt cttcagttgg caaaagctct caattcttta gcttccagtt tcttttctct |
| 8161 | ctttttcttt gttaggtaat taaaggtatg taaacaaatt atctcatgta gcaggggatt |
| 8221 | ttcatgttga gaggaatctt ccgtgtgagt tgtttggtca cacaaataac cctttctcaa |
| 8281 | ttttaggagt ttggattgtc aaatgtaggt ttttctcaaa gggggcatat aactacatat |
| 8341 | tgactgccaa gaactatgac tgtagcacta atcagcacac atagagccac acaattattt |
| 8401 | aatttctaac tctctgtggt ccctagaaaa attccgttga tgtgcttagg ttaaagttct |
| 8461 | gaagataccc gttgtaccct tacttgaaag tttctaatct taagttttat gaaatgcaat |
| 8521 | aatatgtatc agctagcaat atttctgtga tcaccaacaa ctctcagttt gatcttaaag |
| 8581 | tctgaataat aaaacaaatc ccagcagtaa tacatttctt aaacctcaca gtgcatgata |
| 8641 | tatcttttca ttctgatcct gtgtttgcaa aaatatacac atgtatatca tagttcctca |
| 8701 | ctttttattc atttgttttc ctattacctg tagtaaatat attagttagt acatggaatt |
| 8761 | tatagcatca gctaccccca ggaacagcac ctgacaggcg ggggattttt tttcaagttg |
| 8821 | ttctacattt gcataaatta tttctattat tattcatgta tgttatttat ttctgaatca |
| 8881 | cactagtcct gtgaaagtac aactgaaggc agaaagtgtt aggattttgc atctaatgtt |
| 8941 | cattatcatg gtattgatgg acctaagaaa ataaaaatta gactaagccc ccaaataagc |
| 9001 | tgcatgcatt tgtaacatga ttagtagatt tgaatatata gatgtagtat tttgggtatc |
| 9061 | taggtgtttt atcattatgt aaaggaatta aagtaaagga ctttgtagtt gtttttatta |
| 9121 | aatatgcata tagtagagtg caaaaatata gcaaaaataa aaactaaagg tagaaaagca |
| 9181 | ttttagatat gccttaattt agaaactgtg ccaggtggcc ctcggaatag atgccaggca |
| 9241 | gagaccagtg cctgggtggt gcctcctctt gtctgccctc atgaagaagc ttccctcacg |
| 9301 | tgatgtagtg ccctcgtagg tgtcatgtgg agtagtggga acaggcagta ctgttgagag |
| 9361 | gagagcagtg tgagagtttt tctgtagaag cagaactgtc agcttgtgcc ttgaggcttc |
| 9421 | cagaacgtgt cagatggaga agtccaagtt tccatgcttc aggcaactta gctgtgtaca |
| 9481 | gaagcaatcc agtgtggtaa taaaaagcaa ggattgcctg tataatttat tataaaataa |
| 9541 | aagggatttt aacaaccaac aattcccaac acctcaaaag cttgttgcat tttttggtat |
| 9601 | ttgaggtttt tatctgaagg ttaaagggca agtgtttggt atagaagagc agtatgtgtt |
| 9661 | aagaaaagaa aaatattggt tcacgtagag tgcaaattag aactagaaag ttttatacga |
| 9721 | ttatcatttt gagatgtgtt aaagtaggtt ttcactgtaa aatgtattag tgtttctgca |
| 9781 | ttgccatagg gcctggttaa aactttctct taggtttcag gaagactgtc acatacagta |
| 9841 | agcttttttc cttctgactt ataatagaaa atgttttgaa agtaaaaaaa aaaaatctaa |
| 9901 | tttggaaatt tgacttgtta gtttctgtgt ttgaaatcat ggttctagaa atgtagaaat |
| 9961 | tgtgtatatc agatactcat ctaggctgtg tgaaccagcc caagatgacc aacatcccca |
| 10021 | cacctctaca tctctgtccc ctgtatctct tcctttctac cactaaagtg ttccctgcta |
| 10081 | ccatcctggc ttgtccacat ggtgctctcc atcttcctcc acatcatgga ccacaggtgt |
| 10141 | gcctgtctag gcctggccac cactcccaac ttgacctagc cacattcatc tagagatggt |
| 10201 | tcctgatgct gggcacagac tgtgctcatg gcacccatta gaaatgcctc tagcatcttt |
| 10261 | gtatgcatct tgatttttaa accaagtcat tgtacagagc attcagtttt ggctgtggta |
| 10321 | ccaagagaaa aactaatcaa gaatataaac cacattccag gctgctgttt tctctccatc |
| 10381 | tacaggccac acttttactg tatttcttca tacttgaaat tcattctgct attttcatat |
| 10441 | cagggtacag acttataagg gtgcatgttc cttaaaggtg cataattatt cttattccgt |
| 10501 | ttgcttatat tgctacagaa tgctctgttt tggtgctttg agttctgcag acccaagaag |
| 10561 | cagtgtggaa attcactgcc tgggacacag tcttataaga atgttggcag gtgactttgt |
| 10621 | atcagatgtt gcttctcttt tctctgtaca cagattgaga gttaccacag tggcctgtcg |
| 10681 | ggtccaccct gtgggtgcag cacagctctc tgaaagcaag aaccttccta cctattctaa |
| 10741 | cgtttttgcc ctctaagaaa aatggcctca ggtatggtat agacatagca agaggggaag |
| 10801 | ggctgtctca ctctagcaac catccctcca ttacacacag aaagccctct tgaagcaaaa |
| 10861 | gaagaagaaa gaaagaaagc ttatctctaa ggctactgtc ttcagaatgc tctgagctga |
| 10921 | atgctcttgc tcctttccca agaggcagat gaaaatatag ccagtttatc tatacccttc |
| 10981 | ctatctgagg aggagaatag aaaagtaggg taaatatgta acgtaaaata tgtcattcaa |
| 11041 | ggaccaccaa aactttaagt accctatcat taaaaatctg gttttaaaag tagctcaagt |
| 11101 | aagggatgct ttgtgaccca gggtttctga agtcagatag ccattcttac ctgcccctta |
| 11161 | ctctgactta ttgggaaagg gagaactgca gtggtgtttc tgttgcagtg gcaaaggtaa |
| 11221 | catgtcagaa aattcagagg gttgcatacc aataatcctt tggaaactgg atgtcttact |
| 11281 | gggtgctaga atgaaaatgt aggtatttat tgtcagatga tgaagttcat tgtttttttc |
| 11341 | aaaattggtg ttgaaatatc actgtccaat gtgttcactt atgtgaaagc taaattgaat |
| 11401 | gaggcaaaaa gagcaaatag tttgtatatt tgtaatacct tttgtatttc ttacaataaa |
| 11461 | aatattggta gcaaataaaa ataataaaaa caataacttt aaactgcttt ctggagatga |
| 11521 | attactctcc tggctatttt cttttttact ttaatgtaaa atgagtataa ctgtagtgag |
| 11581 | taaaattcat taaattccaa gttttagcag aaaaaaaaaa aaaaaaaa |

SEQ ID NO: 6 Human CDK6 cDNA sequence (transcript variant 2)
(NM_001145306.1) (CDS from positions 518-1498)

| | |
|---|---|
| 1 | cgccgctgtg cccaccccct cgccggagag agtgctggta actccttccc cagagtctga |
| 61 | ttacctgctc cgcgaggccg cggacacgtg cggagagccg actgacactc gcagccccct |
| 121 | cgggaggccc gacgcgactg ggcccctcag gtgcaatgat tctggactga gacgcgcttg |
| 181 | ggcagaggct atgtaatcgt gtctgtgttg aggacttcgc ttcgaggagg gaagaggagg |
| 241 | gatcggctcg ctcctccggc ggcggcggcg gcggcgactc tgcaggcgga gtttcgcggc |
| 301 | ggcggcacca gggttacgcc agccccgcgg ggaggtctct ccatccagct tctgcagcgg |
| 361 | cgaaagcccc agcgcccgag cgcctgagcc ggcggggagc aagtaaagct agaccgatct |
| 421 | ccggggagcc ccggagtagg cgagcggcgg ccgccagcta gttgagcgca ccccccgccc |
| 481 | gccccagcgg cgccgcggcg ggcggcgtcc aggcggcatg gagaaggacg gcctgtgccg |
| 541 | cgctgaccag cagtacgaat gcgtggcgga gatcggggag ggcgcctatg ggaaggtgtt |
| 601 | caaggcccgc gacttgaaga acggaggccg tttcgtggcg ttgaagcgcg tgcgggtgca |
| 661 | gaccggcgag gagggcatgc cgctctccac catccgcgag gtggcggtgc tgaggcacct |
| 721 | ggagaccttc gagcacccca acgtggtcag gttgtttgat gtgtgcacag tgtcacgaac |
| 781 | agacagagaa accaaactaa ctttagtgtt tgaacatgtc gatcaagact tgaccactta |
| 841 | cttggataaa gttccagagc ctggagtgcc cactgaaacc ataaaggata tgatgtttca |
| 901 | gcttctccga ggtctggact ttcttcattc acaccgagta gtgcatcgcg atctaaaacc |
| 961 | acagaacatt ctggtgacca gcagcggaca aataaaactc gctgacttcg gccttgcccg |
| 1021 | catctatagt ttccagatgg ctctaacctc agtggtcgtc acgctgtggt acagagcacc |
| 1081 | cgaagtcttg ctccagtcca gctacgccac ccccgtggat ctctggagtg ttggctgcat |
| 1141 | atttgcagaa atgtttcgta gaaagcctct ttttcgtgga agttcagatg ttgatcaact |
| 1201 | aggaaaaatc ttggacgtga ttggactccc aggagaagaa gactggccta gagatgttgc |
| 1261 | ccttcccagg caggcttttc attcaaaatc tgcccaacca attgagaagt ttgtaacaga |
| 1321 | tatcgatgaa ctaggcaaag acctacttct gaagtgtttg acatttaacc cagccaaaag |
| 1381 | aatatctgcc tacagtgccc tgtctcaccc atacttccag gacctggaaa ggtgcaaaga |
| 1441 | aaacctggat tcccacctgc cgcccagcca gaacacctcg gagctgaata cagcctgagg |
| 1501 | cctcagcagc cgccttaagc tgatcctgcg gagaacaccc ttggtggctt atgggtcccc |
| 1561 | ctcagcaagc cctacagagc tgtggaggat tgctatctgg aggccttcca gctgctgtct |
| 1621 | tctggacagg ctctgcttct ccaaggaaac cgcctagttt actgttttga aatcaatgca |
| 1681 | agagtgattg cagctttatg ttcatttgtt tgtttgtttg tctgtttgtt tcaagaacct |
| 1741 | ggaaaaattc cagaagaaga gaagctgctg accaattgtg ctgccatttg atttttctaa |
| 1801 | ccttgaatgc tgccagtgtg gagtgggtaa tccaggcaca gctgagttat gatgtaatct |
| 1861 | ctctgcagct gccgggcctg atttggtact tttgagtgtg tgtgtgcatg tgtgtgtgtg |
| 1921 | tgtgtgtgtg tgtgtgtgtg tatgtgagag attctgtgat cttttaaagt gttacttttt |
| 1981 | gtaaacgaca agaataattc aattttaaag actcaaggtg gtcagtaaat aacaggcatt |
| 2041 | tgttcactga aggtgattca ccaaaatagt cttctcaaat tagaaagtta accccatgtc |
| 2101 | ctcagcattt cttttctggc caaaagcagt aaatttgcta gcagtaaaag atgaagtttt |
| 2161 | atacacacag caaaaaggag aaaaaattct agtatatttt aagagatgtg catgcattct |
| 2221 | atttagtctt cagaatgctg aatttacttg ttgtaagtct attttaacct tctgtatgac |
| 2281 | atcatgcttt atcatttctt ttggaaaata gcctgtaagc tttttattac ttgctatagg |
| 2341 | tttagggagt gtacctcaga tagattttaa aaaaaagaat agaaagcctt tatttcctgg |
| 2401 | tttgaaattc ctttcttccc tttttttgtt gttgttattg ttgtttgttg ttgttatttt |
| 2461 | gtttttgttt ttaggaattt gtcagaaact ctttcctgtt ttggtttgga gagtagttct |
| 2521 | ctctaactag agacaggagt ggccttgaaa ttttcctcat ctattacact gtactttctg |
| 2581 | ccacacactg ccttgttggc aaagtatcca tcttgtctat ctcccggcac ttctgaaata |
| 2641 | tattgctacc attgtataac taataacaga ttgcttaagc tgttcccatg caccacctgt |
| 2701 | ttgcttgctt tcaatgaacc tttcataaat tcgcagtctc agcttatggt ttatggcctc |
| 2761 | gattctgcaa acctaacagg gtcacatatg ttctctaatg cagtccttct acctggtgtt |
| 2821 | tacttttgtt acctaaataa tgagtaggat cttgttttgt tttatcacca gcacacagat |
| 2881 | tgctataaac tgttactttg tgaattacat ttttatagaa gatattttca gtgtctttac |
| 2941 | ctgagggtat gtctttagct atgttttagg gccatacatt tactctatca aatgatcttt |
| 3001 | tctccatccc ccaggctgtg cttatttcta gtgccttgtg ctcactcctg ctctctacag |
| 3061 | agccagcctg gcctgggcat tgtaaacagc ttttcctttt tctcttactg ttttctctac |
| 3121 | agtcctttat atttcatacc atctctgcct tataagtggt ttagtgctca gttggctcta |
| 3181 | gtaaccagag gacacagaaa gtatcttttg gaaagtttag ccacctgtgc tttctgactc |
| 3241 | agagtgcatg caacagttag atcatgcaac agttagatta tgtttagggt taggattttc |
| 3301 | aaagaatgga ggttgctgca ctcagaaaat aattcagatc atgtttatgc attattaagt |
| 3361 | tgtactgaat tctttgcagc ttaatgtgat atatgactat cttgaacaag agaaaaaact |
| 3421 | aggagatgtt tctcctgaag agcttttggg gttgggaact attctttttt aattgctgta |
| 3481 | ctacttaaca ttgttctaat tcagtagctt gaggaacagg aacattgttt tctagagcaa |
| 3541 | gataataaag gagatgggcc atacaaatgt tttctacttt cgttgtgaca acattgatta |
| 3601 | ggtgttgtca gtactataaa tgcttgagat ataatgaatc cacagcattc aaggtcaggt |
| 3661 | ctactcaaag tctcacatgg aaaagtgagt tctgcctttc ctttgatcga gggtcaaaat |
| 3721 | acaaagacat ttttgctagg gcctacaaat tgaatttaaa aactcactgc actgattcat |
| 3781 | ctgagctttt tggttagtat tcatggctag agtgaacata gctttagttt ttgctgttgt |
| 3841 | aaaagtgttt tcataagttc actcaagaaa aatgcagctg ttctgaactg gaatttttca |
| 3901 | gcattcttta gaattttaaa tgagtagaga gctcaacttt tattcctagc atctgctttt |
| 3961 | gactcatttc taggcagtgc ttatgaagaa aaattaaagc acaaacattc tggcattcaa |
| 4021 | tcgttggcag attatcttct gatgacacag aatgaaaggg catctcagcc tctctgaact |
| 4081 | ttgtaaaaat ctgtccccag ttcttccatc ggtgtagttg ttgcatttga gtgaatactc |
| 4141 | tcttgattta tgtattttat gtccagattc gccatttctg aaatccagat ccaacacaag |
| 4201 | cagtcttgcc gttagggcat tttgaagcag atagtagagt aagaacttag tgactacagc |
| 4261 | ttattcttct gtaacatatg gtttcaaaca tctttgccaa aagctaagca gtggtgaact |
| 4321 | gaaaagggca tattgcccca aggttacact gaagcagctc atagcaagtt aaaatattgt |
| 4381 | gacagatttg aaatcatgtt tgaatttcat agtaggacca gtacaagaat gtccctgcta |
| 4441 | gtttctgttt gatgtttggt tctggcggct caggcatttt gggaactgtt gcacagggtg |
| 4501 | gagtcaaaac aacctacata taaaaagaga aaaagagaaa cttgtccatt tagctttcat |
| 4561 | aagaaatccc atggcaaagg gtaataaaaa ggacctaatc ttaaaaatac aatttctaag |
| 4621 | cacttgtaag aacccagtgg gttggagcct cccactttgt ccctcctttg aagtggatgg |
| 4681 | gaactcaagg tgcaaagaac ctgttttgga agaaagcttg gggccatttc agccccctgt |
| 4741 | attctcatga ttttctctca ggaagcacac actgtgaatg gcagactttt catttagccc |
| 4801 | caggtgactt actaaaaata gttgaaaatt attcacctaa gaatagaatc tcagcattgt |
| 4861 | gttaaataaa aatgaaagct ttagaaggca tgagatgttc ctatcttaaa taaagcatgt |
| 4921 | ttcttttcta tagagaaatg tatagtttga ctctccagaa tgtactatcc atcttgatga |
| 4981 | gaaaactctt aaatagtacc aaacattttg aactttaaat tatgtattta aagtgagtgt |
| 5041 | ttaagaaact gtagctgctt cttttacaag tggtgcctat taaagtcagt aatggccatt |
| 5101 | attgttccat tgtggaaatt aaattatgta agcttcctaa tatcataaac atattaaaat |
| 5161 | tcttctaaaa tattgctttt cttttaagtg acaatttgac tattcttatg ataagcacat |
| 5221 | gagagtgtct tacattttcc aaaagcaggc tttaattgca tagttgagtc taggaaaaaa |
| 5281 | taatgttaaa agtgaatatg ccaccataat tacttaatta tgttagtata gaaactacag |
| 5341 | aatatttacc ctggaaagaa aatattggaa tgttattata aactcttaga tatttatata |
| 5401 | attcaaaaga atgcatgttt cacattgtga cagataaaga tgtatgattt ctaaggcttt |
| 5461 | aaaaattatt cataaaacag tgggcaatag ataaaggaaa ttctggagaa aatgaaggta |
| 5521 | tttaaagggt agtttcaaag ctatatatat tttgaaggat atattcttta tgaacaaata |
| 5581 | tattgtaaaa atttatacta aggtcatctg gtaactgtgg gattaatatg gtcgaaaaca |
| 5641 | aatgttatgg agaagctgtc ccaagcaaac taaattacct gtactttttt cccatttcaa |
| 5701 | gggaagaggc aaccacatga agcaatactt cttacacatg cctaagaacg ttcattgaaa |
| 5761 | aaataaattt ttaaaaggca tgtgtttcct atgccaccaa tacttttgaa aaattgtgaa |
| 5821 | ccttacccaa aaccatttat catgtccatt aagtatattt gggtatataa ttaggaagat |
| 5881 | atttacatgt tccatctcca cagtggaaaa acttattgag gctaccaaag tgtgccaaga |
| 5941 | aatgtaagtc cttagagtaa ttagaaatgc tgttttcctc aaaagcatga gaaactagca |
| 6001 | ttttcatttc ttatttactc cctttctata tcaatgcaat tcacaaccca attttaatac |
| 6061 | atccctatat ctcaagcatt tctatcttgt actttttcag aaaataaacc aaaaataatc |
| 6121 | ctttggtctc tctatcttct gacctttgta agcaacagaa atgtaaaaac agaaggggtc |
| 6181 | caatttttac acgttttttt ctcaagtagc ctttctgggg atttttattt tcttaatgaa |
| 6241 | gtgccaatca gcttttcaaa atgttttcta tttctcagca tttccaggaa gtgataacgt |
| 6301 | ttagctaaat gagtagaagt ggacttcctt caacatattg ttaccttgtc tagccttagg |
| 6361 | aagaaaacaa gagccacctg aaaataaata caggctcttt tcgagcatct gctgaaatac |
| 6421 | tgttacagca atttgaagtt gatgtggtag gaaaggaagg tgacttttct tgcaaaagtc |
| 6481 | tttctaaaca ttcacactgt cctaagagat gagctttctt gttttattcc ggtatattcc |
| 6541 | acaaggtggc acttttagag aaaaacaaat ctgatgaaga ctaaagaggt acttctaaaa |
| 6601 | gagatttcat tctaacttta tttttctgcg catatttaac tctttcctag cacttgtttt |
| 6661 | ttgggatgat taatagtctc tataatgttc tgtaacttca atattttact tgttacctag |
| 6721 | gttctgaaca attgtctgca aataaattgt tcttaaggat ggataataca cccattttga |
| 6781 | tcatttaagt aaagaaagcc tagtcattca ttcagtcaag aaaaaatttt tgaagtaccc |
| 6841 | agttacctta cttttctaga ttaaaacagg cttagttact aaaaaggcag tcctcatctg |
| 6901 | tgaacaggat agtttcgtta gaagtataaa actcctttag tggccccagt taaaacacac |
| 6961 | ataccctctc tgctgctttc aaattcccta gcatggtggc ctttcaacat tgattaaatt |
| 7021 | ttaaaatcct aatttaaaga tcaggtgagc aaaatgagta gcacatcagt aattcagtag |
| 7081 | acaaaacttt tgtctgaaaa attgctgtat tgaaacagag ccctaaaata ccaaaagacc |
| 7141 | aggtaatttt aacatttgtg gaatcacaaa tgtaaattca taagaagctc taattaaaaa |
| 7201 | aaaaaagtct gaagtatatg agcataacaa cttaggagtg tgtctacata cttaactttt |
| 7261 | gaagtttttt ggcaacttta tatacttttt ttaaatttac aagtctactt aaagacttct |
| 7321 | tataccccaa atgattaagt taattttaga ggtcaccttt ctcacagcag tgtcacttga |
| 7381 | aatttagtag ggaaggatat tgcagtattt ttcagtttcc ttagcacagc accacagaaa |
| 7441 | gcagcttatt ccttttgagt ggcagacact cgacggtgcc tgcccaactt tcctcctgag |
| 7501 | tggcaagcag atgagtctca gtaattcata ctgaaccaaa atgccacata cactaggggc |
| 7561 | agtcagaaac tggctgagaa atcccccgcc tcattcgccc ctctgctccc aggaactaga |
| 7621 | gtccagttaa agcccctatg cgaaaggccg aattccaccc cagggtttgt tataacagtg |
| 7681 | gccagtctga accccatttg ctcgtgctca aaacttgatt cccacttgaa agccttccgg |
| 7741 | gcgcgctgcc tcgttgcccc gcccctttgg caggagagag gcagtgggcg aggccgggct |
| 7801 | ggggccccgc ctcccactca cctgccggtg cctgaaatta tgtgcggccc cgcgggctgc |
| 7861 | tttccgaggt cagagtgccc tgctgctgtc tcagaggcat ctgttctgca aatcttagga |
| 7921 | agaaaaatgt ccctagtagc aaacgggtgt cttctgtgca taaataagta caacacaatt |
| 7981 | ctccgaaagt tcgggtaaaa agagatgcgg tagcagctgc cctgtgtgaa gctgtctacc |
| 8041 | ccgcatctct caggcgctaa gctcagtttt tgtttttgtt tttgtttttt taaagaaaag |
| 8101 | atgtataatt gcaggaattt ttttttattt ttttattttc catcattcta tatatgtgat |
| 8161 | ggtgaaagat atgcctggaa aagttttgtt ttgaaaagtt tattttctgc ttcgtcttca |
| 8221 | gttggcaaaa gctctcaatt ctttagcttc cagtttcttt tctctctttt tctttgttag |
| 8281 | gtaattaaag gtatgtaaac aaattatctc atgtagcagg ggattttcat gttgagagga |
| 8341 | atcttccgtg tgagttgttt ggtcacacaa ataacccttt ctcaatttta ggagtttgga |
| 8401 | ttgtcaaatg taggtttttc tcaaaggggg catataacta catattgact gccaagaact |
| 8461 | atgactgtag cactaatcag cacacataga gccacacaat tatttaattt ctaactctct |
| 8521 | gtggtcccta gaaaaattcc gttgatgtgc ttaggttaaa gttctgaaga tacccgttgt |
| 8581 | acccttactt gaaagtttct aatcttaagt tttatgaaat gcaataatat gtatcagcta |
| 8641 | gcaatatttc tgtgatcacc aacaactctc agtttgatct taaagtctga ataataaaac |
| 8701 | aaatcccagc agtaatacat ttcttaaacc tcacagtgca tgatatatct tttcattctg |
| 8761 | atcctgtgtt tgcaaaaata tacacatgta tatcatagtt cctcactttt tattcatttg |
| 8821 | ttttcctatt acctgtagta aatatattag ttagtacatg gaatttatag catcagctac |
| 8881 | ccccaggaac agcacctgac aggcggggga ttttttttca agttgttcta catttgcata |
| 8941 | aattatttct attattattc atgtatgtta tttatttctg aatcacacta gtcctgtgaa |
| 9001 | agtacaactg aaggcagaaa gtgttaggat tttgcatcta atgttcatta tcatggtatt |
| 9061 | gatggaccta agaaaataaa aattagacta agcccccaaa taagctgcat gcatttgtaa |
| 9121 | catgattagt agatttgaat atatagatgt agtattttgg gtatctaggt gttttatcat |
| 9181 | tatgtaaagg aattaaagta aaggactttg tagttgtttt tattaaatat gcatatagta |
| 9241 | gagtgcaaaa atatagcaaa aataaaaact aaaggtagaa aagcatttta gatatgcctt |
| 9301 | aatttagaaa ctgtgccagg tggccctcgg aatagatgcc aggcagagac cagtgcctgg |
| 9361 | gtggtgcctc ctcttgtctg ccctcatgaa gaagcttccc tcacgtgatg tagtgccctc |
| 9421 | gtaggtgtca tgtggagtag tgggaacagg cagtactgtt gagaggagag cagtgtgaga |
| 9481 | gtttttctgt agaagcagaa ctgtcagctt gtgccttgag gcttccagaa cgtgtcagat |
| 9541 | ggagaagtcc aagtttccat gcttcaggca acttagctgt gtacagaagc aatccagtgt |
| 9601 | ggtaataaaa agcaaggatt gcctgtataa tttattataa aataaaaggg attttaacaa |
| 9661 | ccaacaattc ccaacacctc aaaagcttgt tgcatttttt ggtatttgag gtttttatct |
| 9721 | gaaggttaaa gggcaagtgt ttggtataga agagcagtat gtgttaagaa aagaaaaata |
| 9781 | ttggttcacg tagagtgcaa attagaacta gaaagtttta tacgattatc attttgagat |
| 9841 | gtgttaaagt aggttttcac tgtaaaatgt attagtgttt ctgcattgcc atagggcctg |
| 9901 | gttaaaactt tctcttaggt ttcaggaaga ctgtcacata cagtaagctt ttttccttct |
| 9961 | gacttataat agaaaatgtt ttgaaagtaa aaaaaaaaaa tctaatttgg aaatttgact |
| 10021 | tgttagtttc tgtgtttgaa atcatggttc tagaaatgta gaaattgtgt atatcagata |
| 10081 | ctcatctagg ctgtgtgaac cagcccaaga tgaccaacat ccccacacct ctacatctct |
| 10141 | gtcccctgta tctcttcctt tctaccacta aagtgttccc tgctaccatc ctggcttgtc |
| 10201 | cacatggtgc tctccatctt cctccacatc atggaccaca ggtgtgcctg tctaggcctg |
| 10261 | gccaccactc ccaacttgac ctagccacat tcatctagag atggttcctg atgctgggca |
| 10321 | cagactgtgc tcatggcacc cattagaaat gcctctagca tctttgtatg catcttgatt |
| 10381 | tttaaaccaa gtcattgtac agagcattca gttttggctg tggtaccaag agaaaaacta |
| 10441 | atcaagaata taaaccacat tccaggctgc tgttttctct ccatctacag gccacacttt |
| 10501 | tactgtattt cttcatactt gaaattcatt ctgctatttt catatcaggg tacagactta |
| 10561 | taagggtgca tgttccttaa aggtgcataa ttattcttat tccgtttgct tatattgcta |
| 10621 | cagaatgctc tgttttggtg ctttgagttc tgcagaccca agaagcagtg tggaaattca |
| 10681 | ctgcctggga cacagtctta taagaatgtt ggcaggtgac tttgtatcag atgttgcttc |
| 10741 | tcttttctct gtacacagat tgagagttac cacagtggcc tgtcgggtcc accctgtggg |
| 10801 | tgcagcacag ctctctgaaa gcaagaacct tcctacctat tctaacgttt ttgccctcta |
| 10861 | agaaaaatgg cctcaggtat ggtatagaca tagcaagagg ggaagggctg tctcactcta |
| 10921 | gcaaccatcc ctccattaca cacagaaagc cctcttgaag caaaagaaga agaaagaaag |
| 10981 | aaagcttatc tctaaggcta ctgtcttcag aatgctctga gctgaatgct cttgctcctt |
| 11041 | tcccaagagg cagatgaaaa tatagccagt ttatctatac ccttcctatc tgaggaggag |
| 11101 | aatagaaaag tagggtaaat atgtaacgta aaatatgtca ttcaaggacc accaaaactt |
| 11161 | taagtaccct atcattaaaa atctggtttt aaaagtagct caagtaaggg atgctttgtg |
| 11221 | acccagggtt tctgaagtca gatagccatt cttacctgcc ccttactctg acttattggg |
| 11281 | aaagggagaa ctgcagtggt gtttctgttg cagtggcaaa ggtaacatgt cagaaaattc |
| 11341 | agagggttgc ataccaataa tcctttggaa actggatgtc ttactgggtg ctagaatgaa |
| 11401 | aatgtaggta tttattgtca gatgatgaag ttcattgttt ttttcaaaat tggtgttgaa |
| 11461 | atatcactgt ccaatgtgtt cacttatgtg aaagctaaat tgaatgaggc aaaaagagca |
| 11521 | aatagtttgt atatttgtaa taccttttgt atttcttaca ataaaaatat tggtagcaaa |
| 11581 | taaaaataat aaaaacaata actttaaact gctttctgga gatgaattac tctcctggct |
| 11641 | attttctttt ttactttaat gtaaaatgag tataactgta gtgagtaaaa ttcattaaat |
| 11701 | tccaagtttt agcagaaaaa aaaaaaaaaa aaa |

SEQ ID NO: 7 Human CDK6 amino acid sequence (NP_001250.1)

| | |
|---|---|
| 1 | mekdglcrad qqyecvaeig egaygkvfka rdlknggrfv alkrvrvqtg eegmplstir |
| 61 | evavlrhlet fehpnvvrlf dvctvsrtdr etkltlvfeh vdqdlttyld kvpepgvpte |
| 121 | tikdmmfqll rgldflhshr vvhrdlkpqn ilvtssgqik ladfglariy sfqmaltsvv |
| 181 | vtlwyrapev llqssyatpv dlwsvgcifa emfrrkplfr gssdvdqlgk ildviglpge |
| 241 | edwprdvalp rqafhsksaq piekfvtdid elgkdlllkc ltfnpakris aysalshpyf |
| 301 | qdlerckenl dshlppsqnt selnta |

SEQ ID NO: 8 Mouse CDK6 cDNA sequence (NM_009873.3) (CDS from positions
328-1308)

| | |
|---|---|
| 1 | cccgcgctgc gctcatcccc gaggggcccc agcaacctct ccttcgtgaa gactgcacga |
| 61 | gccctgctgt ggaagaaaag tgcagagatt gtgggcagac tatgtaatcg ctgcggaggg |
| 121 | ggaagaggag ggatcggcgc tctcctgcgg cggcggcgcg gcgactcggc taggcggagt |
| 181 | ttcgcagcgg ctgcgcccgg cttgcacccg cgggcgagaa ggtcggtccg tctagcccgg |
| 241 | cggccgcgag tccgactccc cgaggcgtgt aaggcagcga gtgagcaccc cggttccact |
| 301 | gtgccgcacc cgcagcctga agccagcatg gagaaggaca gcctgagtcg cgccgatcag |
| 361 | cagtatgagt gcgtggcgga gatcggcgaa ggcgcctatg ggaaggtgtt caaggcccgc |
| 421 | gacctgaaga acggcggccg cttcgtggct ctgaagcgcg tgcgagtgca gaccagtgag |
| 481 | gagggcatgc cgctctccac catccgcgag gtggcggtgc tgaggcacct ggagaccttc |
| 541 | gagcacccca acgtggtcag gttgtttgat gtgtgcacag tgtcacggac ggacagagaa |
| 601 | accaagctta cactagtgtt tgagcatgtt gatcaagact tgaccactta cttggataaa |
| 661 | gttccagagc ccggcgtacc cacagaaacc ataaaggata tgatgtttca gcttctccga |
| 721 | ggtctggact ttcttcattc tcacagagta gtgcatcgtg atctgaaacc gcagaacatt |
| 781 | ctggtgacca gcagtggaca gataaagctg gctgactttg gccttgcccg catctatagt |
| 841 | tttcagatgg cccttacctc ggtggtcgtc acgctgtggt accgagcccc agaagtcctg |
| 901 | ctccagtcca gctatgccac ccctgtggac ctctggagtg tcggttgcat ctttgcagaa |
| 961 | atgtttcgca gaaagcctct ttttcgtgga agttcagacg tggatcaact aggaaaaatc |
| 1021 | ttggacatca ttggactccc aggagaggaa gactggccta gggacgtggc ccttccccgg |
| 1081 | caggcttttc attccaaatc tgctcaaccc atcgagaagt ttgtgacaga tattgacgaa |
| 1141 | ctaggcaaag acctacttct gaaatgcctg acgtttaatc cagctaaaag gatatccgcc |
| 1201 | tacggcgccc tgaatcaccc gtacttccaa gatctggaga gatacaagga caacctgaac |
| 1261 | tctcacctgc catccaacca gagcacctcg gagctgaaca cagcctgagg ttccacgggg |
| 1321 | atgcccatga gctcgtcatc tgaacacatt ggcggctgcg agtcccctaa gcaagcctct |
| 1381 | cagagcagtt gaagattgct ggctgccaac cttctggctg ccagcttctg ggtgggctct |
| 1441 | gccttaccaa ggaaaccacc tagtttactg ttcagagatc aatgcaaggg tgattgcagc |
| 1501 | tttatgttcg tttgtacact tgtttgtttt gtctgtttgt ttcaagaacc tggaaaactt |
| 1561 | ccagaagaag agaagctgct gaccaattgt gctgccattt cgttttctaa ccttgaatgc |
| 1621 | tgccagtgta gggtgggaat ccaggcccag ctgagttatg atgtaatccg cctgcagctg |
| 1681 | ctgggcctgc tttggtactt gtgagtgtgt gtgcatgcgt atgtgtgtgt aagagagaag |
| 1741 | aggaggggag agaaagaccc ctgatctcgt caagtgttac tttttttttg tagaaaacaa |
| 1801 | gaataattga gttttaaaga gtagaggtga ctgatagtaa gaagggcttg ttcagtgaaa |
| 1861 | ggtgattcac aatggagtct tgttaggaag gttggaccta agtcctcaga gttgccttcc |
| 1921 | tgtccaaaag cttttgctag cagtaaacaa taaaggttta gatgccacaa aaaatggggg |
| 1981 | gaaccacaat attttttaag agacttttta aggcatacat cttctattta ctctttggaa |
| 2041 | agctgaactt aatgtgtccc aggccctata tatagtacag tatgtactta attgtttctt |
| 2101 | tggggaaaga tgctataagt atcttattac ttgcaataca tttaaggagt gagtgtacct |
| 2161 | cagataggtt ttaaagatag agagcacctg ttttctggtg tgagatgtta tcattttctt |
| 2221 | cacgtctctt gataccttga taccttgtca ccttagggaa tcacttcctg ctctgactag |
| 2281 | aggcgggaat accatctagc tgtctccacc acccaccatg gcgcatctgc cttgtgctgc |
| 2341 | cttgtgtagt gcgaagctct caaccaccag cacttctaat tcattttcct gccactgcct |
| 2401 | ggctaacgac agatggccca gctgccccaa tcccacaccc gcttgcacgc ttaccgtctt |
| 2461 | tcaccgaatg ctttgggcgt aggctcccat tccgaaaccc taacagtatc cccttgtgcc |
| 2521 | tttgtaatac agtcttcccc ctgccgcagc tgaggtcacc taggcagtga agagtgcttg |
| 2581 | ttctgtgtgt gtatagacta ctaccgactg tcacttggtg tttcctatct ttaagtgtat |
| 2641 | gttgtcagtg taatgtctga ggaaatgtct tttcctctct tctagagata actacttact |
| 2701 | ctctaaagtg atctctctgt ctgtccgcag gatgtgtttc tgggtttttg ttcttttttt |
| 2761 | tttttttttt ttttttttgt ctgaggcctc atgtcagccc tgctttctgc agagccagcc |
| 2821 | taccctgggc attataaaca actgtacctt actgttttct ttcagtcctt tacattctgt |
| 2881 | gccacctttg ccttattata tccgtggctt agtgctaagc tggcactact ccatagaagg |
| 2941 | tgtagaaggc agctttcggg aagcgtagct gagttttgtt tgaatcgtgt ttgagcacat |
| 3001 | ttaagtaaac gctagatgat gcttcactgt agagctttga aagactgcct gatgtttcac |
| 3061 | acggcagtgt aggctccaga tggtgtttgt atgttatgga ccttactgtg tttctcgtag |
| 3121 | ctgaacaatg catgtggtta tttgtttgtt tgttttttgg tttctctgtg tagccctggc |
| 3181 | tgtcctggaa ctcactctgt agaccaggct ggcctcgaat tcagaaatgc gcctgcctct |
| 3241 | gcctcccaag tgctgggatc aaaggcgtgc gccaccacgc ccggctttgc atatggttat |
| 3301 | ctgtatggag aagaattaga tgtctttcct gagagctttg gaggttggga atcatcttct |
| 3361 | tctgtttatt tttattttta acattacatt acattcttct gattcagtat cgcaagaaag |
| 3421 | gattttttct tttttcaaaa acaaaacttt aagaagccag aggctaggct gagaatgttc |
| 3481 | tgcgctcgtc ctcacagctc tgctgccgac actgccaacg ctcgagacag tcagcctgga |
| 3541 | acctctggga caaggtctac tcgaggtctc acttggacag cgagttctgc cttgtcattg |
| 3601 | aacaaggtcc aaaatacaga catttttgct agggcctaga aatcgaccat aaaactcact |
| 3661 | gcattgaggc ctaatccatt ctatattaac atccatgtga taaagtgaaa ataattttga |
| 3721 | gggttttgct ttcgccaaaa aaaaaaaaga aagaaagaaa aaaaaagaaa ggaagaaaaa |
| 3781 | gaaaaaagaa aggattgttt tccttagctt tgctcaagaa aaatacagct atcctaaact |
| 3841 | ggaatcttcc cgtattcttt agagtcttaa gtatagagtt taacttcact tttgtcagct |
| 3901 | gtcttgagct tatttctaag tagttttgtt ttggcttttg gtgagtgagg tagtgggagg |
| 3961 | ttaattttgg ggtctttttt ttttttggct ttgtttgtgt gtgtgtgtgt gtgtgtgtgt |
| 4021 | ttatgtgttt ggcttttggg tttggttggt tagttgttta gtttgttttt ttgttgttgt |
| 4081 | ttaatttgct ttttctaggc agggtttctc tatgtggccc tggctgtcct agaacttgct |
| 4141 | ctgtagacca ggctagcctc aaactcagag atccacttgc ctctgtctcg agtgtgcttg |
| 4201 | aactaaaggc atgagctacc ctcctcagcc cactccaaac tttgagtaat atttttaaat |
| 4261 | catagacatg ttggtattag aaacatggcg tattgtggtg gatggtagag aaagggcgcc |
| 4321 | gcaacctctc aggacatcat atgtctggcc agctcttcat tcatataggt gctctctgga |
| 4381 | tatgtcttta catcagactc acacctctga aacccggttt tcacacaagg agacttgcca |
| 4441 | ttggggtctt tagtagccca gggaaacata gagtaaactg aagagcttac tgtgtgccgc |
| 4501 | ttatccttag atggcctcat ccagtcacag gtctttgtca gaatctaacc attgattcac |
| 4561 | tgaggagacc ctactgccct gaggttccac tgaagcagct tttagtgaga ctagggcaag |
| 4621 | aatataccga atagcttctg ttcaaggttt ggttccagag actctaccat ttacaggact |
| 4681 | ggctggagtt acaacaacct cttaaagagg gtagggcggg ttctccagtt agacccccta |
| 4741 | acctcacagg tgagtagtcc taacctgaca gccttagaaa cacagttgcc gagtacctga |
| 4801 | gagcacccag tggcccgccg cctcctattc agcccctctt taaagacgac agacggttaa |
| 4861 | gccatggaga agctgttcag gtagcacggg gccatttctg cccccagctt gtcatgtttt |
| 4921 | ttctctcagg acccactctc tgaccagtag acttgaattt agccccaagt gatttactaa |
| 4981 | actcagctca gaagctcact taaaaatagg acctccacat ggtcgtgcta agtaaaaatg |
| 5041 | aagaccccaa gttgcacagg gtgctgctat ctcaaagaca atactttcct ttgaatagaa |
| 5101 | agctgtgccg tccaccctgc aatgggtgct agccaacttg cttgagaaag tcaggagcaa |
| 5161 | gcattttgag ttttaaagaa tgtcctgaac ttgtctttta agtaaacata gctgctttat |
| 5221 | taaaagctgt gactactaaa gccagtcaca gccatcgtca ctgtgggagt taaatcatgt |
| 5281 | caactttcaa acatcacagt tgtttcaatc ttctgtccaa agaactcctc ttctggagaa |
| 5341 | aacctgattt tggttttggc tttttgtctt gtcttgtttt gtttttcctg agatggggtc |
| 5401 | tctctgtgaa gccctggcta tcctggaact cagtgtgtag accaggctgg ccttgaactc |
| 5461 | atgtagatct gcctgctaaa actaaagaca tgcaccacca tgacagctga ctatttttta |
| 5521 | tgatgagcac agaagtggct cacattttct taaagcacac ttttgtcaca tagtagagtc |
| 5581 | taggaaagaa taacattgaa aatccccatc ccagtgtgac acttgtgtaa gcacatatgg |
| 5641 | atagccattg gaagaataca tgcttgcttg taacatgaaa ttaaagacat tgtataattt |
| 5701 | cttaatcttt aaaaattacc aaaaaagtgg caacagtatg tttaaggtta ttttaatttt |
| 5761 | tacataaatt gttaaccata tttgtttgcc tttaagtata ttctaagaat tataattaag |
| 5821 | tataattata agtcatcccg cacattcagc acttatggac taggtatgtc aaaatacaca |
| 5881 | aattctgtat ttcttgtttc ataggaagaa agcagccatc tgaagctcta ctcatatata |
| 5941 | tgcctaagaa tatctataca aaattaaagt ttaaaaggcc tttgcctctc gtgcagctga |
| 6001 | gaatagacaa cttataagcc ttatccaaaa accatgataa agggatcatg tgcatcctta |
| 6061 | caaaaacccc ttatcctgtg catacttaca tccaagtaca tcctaaggaa ggtgtccgtg |
| 6121 | cattcatctg ccacagtggg gaagctgcct ggtgcaaggt gtgagtcctg gaatgattag |
| 6181 | tgatagtggt ctcaaatgag gacaccttcg tctcccacca gtttccttcc tatggacagg |
| 6241 | ctcagtccag ttatacaggc caggaggggc tggtgcccac agcctcccgc ctctgcaagg |
| 6301 | taggattgca tatctcaaac atctctaact cagtttcggc tttgttggtc ttgtttaagg |
| 6361 | aaataagcca gtaagccaaa actatttttg ttgttgttct cttgtcttct gaaagagagg |
| 6421 | ggatatcaat tttaaatgtt tctctcaatt tgatctttca aatttttcta ataaagtgcc |
| 6481 | aaattgctgc tgcttcttct tcttctttct tcttcttctt cttcttcttc ttcttcttct |
| 6541 | tcttcttctt cttcttcttc ttcttcttct tcttcttcat gttttctgct agggttttct |
| 6601 | tgttttgttt tggtttcagt ttgttttatt ttcagcaagt taggtaactt gagaggactt |
| 6661 | ccactaggat gttcacacct ctgtgctctg gtcctctgac ccctccaaaa aatagccacc |
| 6721 | tgggaataaa tgcaagctcc ccccctccag catctaccaa aatactgttg cagcaattcc |
| 6781 | aagttcaaat ggtggggatg gaaggtagtt tggagttggg ttgggttggg ttgggttagg |
| 6841 | ttggacagaa gtcagtttta aaattcacag cgccctaagg tcagtgtggg gtttattcct |
| 6901 | ctatcccaat gtgtttcaca aggtggcgct ttcagagaaa actctaaacc tgaccaagac |
| 6961 | tgagaggtat tctagagaca gtacttgtaa ctattttcct gcacatctgt aactatcacc |
| 7021 | tagcatattc cttcagacaa ttaacagcct tcataatatt ctacagctta tatgttttgc |
| 7081 | tttttagcta ggttctgagt gactaagtta ggcttgagag tgatgtttaa gcaataaaga |
| 7141 | aggcctttac ttaagagaca tttttgatgt acccaaataa cctgaaattc taggacatac |
| 7201 | aactaaaaac ttccctgaag ccggccctcg tctgtgagca gcacggtttc attagaaatg |
| 7261 | gcaaaactcc ttgcggggtt gggagtgggg gacagtccta actctcacag cccttgacat |
| 7321 | gtctgctgcg gttcacccct cactatccat gaccttttga cactgtcatt tctaaactca |
| 7381 | agtgaacaaa ggaagcactg catctgtaac tgagtatgca gaccattttc taaagaacca |
| 7441 | cagtgctcga caaatactgg aagaactggt atgttttaca cgcacataaa ctacaagtgc |
| 7501 | aaactcttac attccaattc ctaaattaca gttatatagt acatatgaac attacagttt |
| 7561 | agacaggaac ctgcataagg ctttggtttt tctactctcg aggtcacttt tatactacat |
| 7621 | tttaaattca cacttttctc agagacccag ccccctgagc caatagtgac agtacactgg |
| 7681 | gatgaacaga cacagtacca caccatggca gaaagcagtt tattccttat gagtcacaca |
| 7741 | cacacacaca cacaccctgc ctgacttcac acacacacac acacactgcc tgacttcaca |
| 7801 | cacacacacc ctgcctgact tcactccaga aaggctggca gggaggtttc agtcatttac |
| 7861 | acagaccagg tgctcaagca ttgagaaatc gattctttca accctctgct ctcactgaag |
| 7921 | cagatctcag gttaagcccc tcagtgaaag gccaagggtc ccctgggtct gccaccattc |
| 7981 | tggagttcct gagcctgacc ccacccctac tcctgctgaa tgacaggggg cggggcagcg |
| 8041 | gagccaggcc aagcaggata ttgtgggagg accagtgacc tgctgcacct cagccatctg |
| 8101 | ttgtgccatc ttaggaagca gggtgctcga gaaacacaga gctgtcctct gagctacaat |
| 8161 | aactacagtt tgctcactag gctagctagc tctggtgtag agatgcaagc cctatatgaa |
| 8221 | atgtctccca ctcaaagttt tgctgggggg tgggggttga gatgtataat tgcaggagaa |
| 8281 | attctgactt tactgttttc tatctgatac atccatggtg gggaaaagat atgaaactgt |
| 8341 | ctcagtttga aaagctcttc tacttcaatt ggcaaacgca tggagttcgt tactttctac |
| 8401 | tttcttcttt tttctccttt agtgagcaaa ttattccttg aacagaaaga ggattttccc |
| 8461 | atttagagga agcttctgtg tgaattgtca ggtcacacaa ataatgccat tatattgttg |
| 8521 | agtgtagggc tttggttttt caggggacac atagccaagc agtgatggtc aggaattatg |
| 8581 | tctcactcta tcatttctta ctcaagcagg gttccagcca gattctattg tgagctgagg |
| 8641 | ttagtcgggg gaccctgtgc tctacttgaa gtttcataat ctttagttct atgaaatgca |
| 8701 | ataacatata ttagccagca atattcctgt ggtcgccagc atctctgagt ttgaccttca |
| 8761 | gtctctggat ggttaaaata gatcccagca gtaatccatt tcttgacatc agtgcttaag |
| 8821 | tcaagtatct ttccactctg gtcctatttg caacaatgta ttcatgtatg tttcatttcc |
| 8881 | tttattcatt attttcctat gaacagtgat aaatgtatta gcacattgaa ttaatagcat |
| 8941 | aatagaaaga gcacttagca gatggggatt ttttatcaag tcccacattt acataagtta |
| 9001 | tttctattat tattcatgta tattatttat tgctaagcct cacaccattc ctgtgaaagt |
| 9061 | gcaactaaag gcagatatta gggcttggat ttaatgttcc ttatcatggt cttgaacaaa |
| 9121 | aaattcagtg atgcccccaa ataagctgcc tgcgttttta acacgactgc tagatttcag |
| 9181 | taggttaagt gtaacacttt tggtgtctag gtgtcttttt acattaaaag aggcaaagca |
| 9241 | gcagaactct gtagctattg cttctggcag agatttgttt agcatagtgt ggtattaatt |
| 9301 | atagcaaatg ttaaggtagc atgtagaggt tgccttcaat aagggactgt gccaggtggc |
| 9361 | cagcaaagtg gattctaggc agagcaggcg tctaggtgct gcctcctgtc tggttgtgtg |
| 9421 | aagagagctg ctccgagcaa tgtagtgccc ccgtgccccc gtaggtgtcc tgggcagcgg |
| 9481 | tgggaacacg tggtactgta gagaagggat cagcgtgagg ggtgttctgt aggaacagaa |
| 9541 | ctggtagcgt atgccttgag gcttcctgcc cgtgttttca gattccaggc atacattctg |
| 9601 | tatacagaag cagtccatcc tagcaataaa acctaggaat ccccaacaac tcaaaaggca |
| 9661 | gttgccctgc tgtggtttct gtcctgagct gaaggttaac gggcaagtgt taggcacaga |
| 9721 | agagcatcgt gtgctggggt gccgtgccac ggcccagggt gcgtgttagc agtagagact |
| 9781 | gttacagggt tgtcccttct aggtgcgtag aagtatatgg cttcactgta aagtgtgtat |
| 9841 | tgtttctgta ttcccacagg gacttgttaa agcttctaga ggcttcagca aagctgtaga |
| 9901 | ccacagtaag ctgctttttg tttgacttat tcttagacta ggaaatgttt aaaggttaaa |
| 9961 | aaaaaaaaaa aaagaatctt catttagaca ctgaactcgc tattttctgg actgaaatca |
| 10021 | tggtcctaga actgtagaaa tcacacccat tagatgctaa tctaggttgt gtaagccagc |
| 10081 | cccaagagtc tagcatcccc acactgtatc tgcatcttcc tgcatcttac cttccttccc |
| 10141 | gaaggtgctc cctaccacta ccctgacctg tccctttggt gcttcccatc tccttggctg |
| 10201 | aaagttaggc atcaagcatg tgatgagaaa tactttcagc accaagtatg cctgcctagg |
| 10261 | cctagccacc gcccccactt tgcctttggc tagccatagt caacaagaga taatacctgg |
| 10321 | tgctgggcac agaatgaacc catgatgtca gtcagaaaag ccctcaagca tatttctcct |
| 10381 | gtacgtcctg atctttaaac ccagtcatta tgcagagcgt tcgctgttgg ctatagtccc |
| 10441 | aggttaaaaa aaaaaaaaac attccaggct gctgtaggct gtccatatgc agtctctgct |
| 10501 | tttaccttgc tttttcatac ttgaaattcg ttctgccatt ttgatttcac ggtgcagaca |
| 10561 | tcagggtaca cgttccctct gggtgcataa ttgctctcat tccgtttgct cacactgcta |
| 10621 | caggatgctc tgttttggtg ctttgggttc agcgggtcaa aaagcactgt gggaaatcac |
| 10681 | tgcctagaac acagtcttaa aagcatttgg gcaggagtct tggtgttgat gtcgcttctg |
| 10741 | ctcttctctg tgccctccag atggagttac caggttggcc tgccaggtcc cccactccca |
| 10801 | cccccaagac ctttctctga atggcctgtg aaccctgtgc tgtatccaag ggtgaatgtg |
| 10861 | acgtatctcc tctggcgacc ctacctttcc tggttactca ccatcagaaa caacctaggt |
| 10921 | tacatatagc aaactctaca cagccttcct gagggaaagc ataaaaggat atctctaagg |
| 10981 | ttgctcttca aagccctgag ctgaaggcct ttgctcccgt ccttggagct gatgaaaata |
| 11041 | ctggtcaatt tatccaagcc cttcccactt acaagaatgg gaaagtagag gtttgtgtcg |
| 11101 | ataccatcta aagaccacaa cttctagcca tagggtattt catatatgtc cattttcaaa |
| 11161 | agcagctcag gtgagtggtg actccaggct ccatgcaggc atcttaacat gggacttccc |
| 11221 | tagagagagc tacagtgttc attctatgtc acagggcagg agaggagacc ggcaggaaat |
| 11281 | ttggagggta acagatcaat actcctttgg aaactggatg tcttactggg tgctagaatg |
| 11341 | caaatttatg tatttattgt ctggtcattg aattcattgc ctttcaaaac cattgttcaa |
| 11401 | atgtcactat cattggcctc acttgtatgc cagccgaatt ggttgaaagc aaaccagaga |
| 11461 | atggtttgtg catttgcctt accatttgta cttcttacaa taaaaatgtc agtagcaaat |
| 11521 | aaaaaaaaaa aaaa |

SEQ ID NO: 9 Mouse CDK6 amino acid sequence (NP_034003.1)

| | |
|---|---|
| 1 | mekdslsrad qqyecvaeig egaygkvfka rdlknggrfv alkrvrvqts eegmplstir |
| 61 | evavlrhlet fehpnvvrlf dvctvsrtdr etkltlvfeh vdqdlttyld kvpepgvpte |
| 121 | tikdmmfqll rgldflhshr vvhrdlkpqn ilvtssgqik ladfglariy sfqmaltsvv |
| 181 | vtlwyrapev llqssyatpv dlwsvgcifa emfrrkplfr gssdvdqlgk ildiiglpge |
| 241 | edwprdvalp rqafhsksaq piekfvtdid elgkdlllkc ltfnpakris aygalnhpyf |
| 301 | qdlerykdnl nshlpsnqst selnta |

*Included in Table 1 are RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof Such nucleic acid molecules can have a function of the full-length nucleic acid as described further herein.
*Included in Table 1 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1, or a portion thereof. Such polypeptides can have a function of the full-length polypeptide as described further herein.

### II. Subjects

In one embodiment, the subject has a condition that would benefit from upregulation of an immune response. The subject can be treated at least one CDK4 and/or CDK6 inhibitor, either alone or in combination with an immunotherapy, such as an immune checkpoint inhibition therapy. The subject can be a mammal (e.g., mouse, rat, primate, non-human mammal, domestic animal such as dog, cat, cow, horse), and is preferably a human. The term “subject” refers to any healthy animal, mammal or human, or any animal, mammal or human afflicted with an immune disorder. The term “subject” is interchangeable with “patient.”

In another embodiment of the methods of the invention, the subject has not undergone treatment, such as chemotherapy, radiation therapy, targeted therapy, and/or anti-cancer therapy (e.g., at least one CDK4 and/or CDK6 inhibitor, either alone or in combination with an immunotherapy therapy, such as an immune checkpoint inhibition therapy). In still another embodiment, the subject has undergone treatment, such as chemotherapy, radiation therapy, targeted therapy, and/or anti-cancer therapy (e.g., at least one CDK4 and/or CDK6 inhibitor, either alone or in combination with an immunotherapy, such as an immune checkpoint inhibition therapy). In yet another embodiment, the subject is immunocompetent or immune-incompetent. “Immunocompetent” subjects are those subjects comprising immune cells and immune function required to establish a normal or desired immune response following exposure to an antigen. “Immuno-incompetent” subjects are those subjects lacking one or more immune cell types or lacking an immune function thereof to establish a normal or desired level of at least one immune response following exposure to an antigen. Immuno-incompetent subjects are more susceptible to opportunistic infections, for example viral, fungal, protozoal, or bacterial infections, prion diseases, and certain neoplasms. “Immunodeficient” subjects are subjects in which no native host immune response may be mounted, such as is the case with severe combined immunodeficiency (SCID) mice. “Immunocompromised” subjects have at least one substantially reduced immunological function relative to immunocompetent subjects. In either case, reduction in or absence of immunological function and/or cell types can arise from many different and well-known manners. For example, hematopoietic stem cells (HSCs) that give rise to all immune cells are any project thereof can be negatively affected in development, function, differentiation, survival, and the like.

In some embodiments, the subject is in need of an upregulated immune response, such as by reducing Tregs to remove inhibition of immune responses. Agents that upregulate immune responses can be in the form of enhancing an existing immune response or eliciting an initial immune response. Thus, enhancing an immune response using the subject compositions and methods is useful for treating cancer, but can also be useful for treating an infectious disease (e.g., bacteria, viruses, protozoa, helminth, or other parasites), asthma associated with impaired airway tolerance, and an immunosuppressive disease. Exemplary infectious disorders include viral skin diseases, such as Herpes or shingles, in which case such an agent can be delivered topically to the skin. In addition, systemic viral diseases, such as encephalitis might be alleviated by systemic administration of such agents. As described below, respiratory infections, such as influenza and the common cold, can be treated by respiration-based administration, such as intranasal, pulmonary inhalation, lung deposition, and related routes well-known in the art. In certain embodiments, the subject has had surgery to remove cancerous or precancerous tissue, such as by blood compartment purification. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

The methods of the invention can be used to determine the responsiveness to anti-cancer therapy (e.g., at least one CDK4 and/or CDK6 inhibitor, either alone or in combination with an immunotherapy, such as an immune checkpoint inhibition therapy) of many different cancers in subjects such as those described above.

### III. Sample Collection, Preparation and Separation

In some embodiments, biomarker presence, absence, amount, and/or activity measurement(s) in a sample from a subject, such as baseline Treg numbers, Treg ratios, biomarker expression level, interferon or interferon signaling pathway gene expression, CDK4, CDK6, interferons, ISGs, immune checkpoints, DNMT1, STAT1, STAT2, IRF2, IRF6, IRF7, IRF9, NLRC5, OAS1, OAS2, IFIT1, IFIT2, IFIT6, BST2, SP100, RSAD2, CXCL9, CXCL10, CXCL11, Icam1, Vcam1, IL-29, IL-28a, IL-28b, ERV3-1, ERVK13-1, RIG-1, LGP2, MDA5, and the like, is compared to a predetermined control (standard) sample. The sample from the subject is typically from a diseased tissue, such as cancer cells or tissues, but can be any tissue of interest, such as serum or other bodily sample described herein. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples, such as the normal copy number, amount, or activity of a biomarker in the cell or tissue type of a member of the same species as from which the test sample was obtained or a non-diseased cell or tissue from the subject from which the test samples was obtained. As described herein, a “pre-determined” biomarker amount and/or activity measurement(s) may be a biomarker amount and/or activity measurement(s) used to, by way of example only, evaluate a subject that may be selected for treatment, evaluate a response to an anti-cancer therapy (e.g., at least one CDK4 and/or CDK6 inhibitor, either alone or in combination with an immunotherapy, such as an immune checkpoint inhibition therapy), and/or evaluate a response to a combination anti-cancer therapy (e.g., at least one CDK4 and/or CDK6 inhibitor, either alone or in combination with an immunotherapy, such as an immune checkpoint inhibition therapy). A pre-determined biomarker amount and/or activity measurement(s) may be determined in populations of patients with or without a condition of interest, such as cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., biomarker expression normalized to the expression of a housekeeping gene, or gene expression at various time points).

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present invention the change of biomarker amount and/or activity measurement(s) from the pre-determined level is about 0.5 fold, about 1.0 fold, about 1.5 fold, about 2.0 fold, about 2.5 fold, about 3.0 fold, about 3.5 fold, about 4.0 fold, about 4.5 fold, or about 5.0 fold or greater. In some embodiments, the fold change is less than about 1, less than about 5, less than about 10, less than about 20, less than about 30, less than about 40, or less than about 50. In other embodiments, the fold change in biomarker amount and/or activity measurement(s) compared to a predetermined level is more than about 1, more than about 5, more than about 10, more than about 20, more than about 30, more than about 40, or more than about 50.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes.

Separation and purification in the present invention may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE.

Separation and purification techniques used in the present invention include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### IV. Biomarker Nucleic Acids and Polypeptides

One aspect of the present invention pertains to the use of isolated nucleic acid molecules that correspond to biomarker nucleic acids that encode a biomarker polypeptide or a portion of such a polypeptide, such as CDK4, CDK6, interferons, ISGs, immune checkpoints, DNMT1, STAT1, STAT2, IRF2, IRF6, IRF7, IRF9, NLRC5, OAS1, OAS2, IFIT1, IFIT2, IFIT6, BST2, SP100, RSAD2, CXCL9, CXCL10, CXCL11, Icam1, Vcam1, IL-29, IL-28a, IL-28b, ERV3-1, ERVK13-1, RIG-1, LGP2, and MDA5. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or substantially free of chemical precursors or other chemicals when chemically synthesized.

A biomarker nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

Moreover, a nucleic acid molecule of the invention can comprise only a portion of a nucleic acid sequence, wherein the full length nucleic acid sequence comprises a marker of the invention or which encodes a polypeptide corresponding to a marker of the invention. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a biomarker nucleic acid sequence. Probes based on the sequence of a biomarker nucleic acid molecule can be used to detect transcripts or genomic sequences corresponding to one or more markers of the invention. The probe comprises a label group attached thereto, e.g., a radioisotope, a fluorescent compound, an enzyme, or an enzyme co-factor.

A biomarker nucleic acid molecules that differ, due to degeneracy of the genetic code, from the nucleotide sequence of nucleic acid molecules encoding a protein which corresponds to the biomarker, and thus encode the same protein, are also contemplated.

In addition, it will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

The term “allele,” which is used interchangeably herein with “allelic variant,” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. For example, biomarker alleles can differ from each other in a single nucleotide, or several nucleotides, and can include substitutions, deletions, and insertions of nucleotides. An allele of a gene can also be a form of a gene containing one or more mutations.

The term “allelic variant of a polymorphic region of gene” or “allelic variant”, used interchangeably herein, refers to an alternative form of a gene having one of several possible nucleotide sequences found in that region of the gene in the population. As used herein, allelic variant is meant to encompass functional allelic variants, non-functional allelic variants, SNPs, mutations and polymorphisms.

The term “single nucleotide polymorphism” (SNP) refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of a population). A SNP usually arises due to substitution of one nucleotide for another at the polymorphic site. SNPs can also arise from a deletion of a nucleotide or an insertion of a nucleotide relative to a reference allele. Typically, the polymorphic site is occupied by a base other than the reference base. For example, where the reference allele contains the base “T” (thymidine) at the polymorphic site, the altered allele can contain a “C” (cytidine), “G” (guanine), or “A” (adenine) at the polymorphic site. SNP's may occur in protein-coding nucleic acid sequences, in which case they may give rise to a defective or otherwise variant protein, or genetic disease. Such a SNP may alter the coding sequence of the gene and therefore specify another amino acid (a “missense” SNP) or a SNP may introduce a stop codon (a “nonsense” SNP). When a SNP does not alter the amino acid sequence of a protein, the SNP is called “silent.” SNP's may also occur in noncoding regions of the nucleotide sequence. This may result in defective protein expression, e.g., as a result of alternative spicing, or it may have no effect on the function of the protein.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, a biomarker nucleic acid molecule is at least 7, 15, 20, 25, 30, 40, 60, 80, 100, 150, 200, 250, 300, 350, 400, 450, 550, 650, 700, 800, 900, 1000, 1100, 1200, 1300, 1400, 1500, 1600, 1700, 1800, 1900, 2000, 2200, 2400, 2600, 2800, 3000, 3500, 4000, 4500, or more nucleotides in length and hybridizes under stringent conditions to a nucleic acid molecule corresponding to a marker of the invention or to a nucleic acid molecule encoding a protein corresponding to a marker of the invention. As used herein, the term “hybridizes under stringent conditions” is intended to describe conditions for hybridization and washing under which nucleotide sequences at least 60% (65%, 70%, 75%, 80%, preferably 85%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a polypeptide of the invention that contain changes in amino acid residues that are not essential for activity. Such polypeptides differ in amino acid sequence from the naturally-occurring proteins which correspond to the markers of the invention, yet retain biological activity. In one embodiment, a biomarker protein has an amino acid sequence that is at least about 40% identical, 50%, 60%, 70%, 75%, 80%, 83%, 85%, 87.5%, 90%, 91%, 92%, 93%, 94%, 95%, 96%, 97%, 98%, 99% or identical to the amino acid sequence of a biomarker protein described herein.

An isolated nucleic acid molecule encoding a variant protein can be created by introducing one or more nucleotide substitutions, additions or deletions into the nucleotide sequence of nucleic acids of the invention, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In some embodiments, the present invention further contemplates the use of anti-biomarker antisense nucleic acid molecules, i.e., molecules which are complementary to a sense nucleic acid of the invention, e.g., complementary to the coding strand of a double-stranded cDNA molecule corresponding to a marker of the invention or complementary to an mRNA sequence corresponding to a marker of the invention. Accordingly, an antisense nucleic acid molecule of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a polypeptide corresponding to a selected marker of the invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The present invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the invention can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The present invention also encompasses nucleic acid molecules which form triple helical structures. For example, expression of a biomarker protein can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

Another aspect of the present invention pertains to the use of biomarker proteins and biologically active portions thereof. In one embodiment, the native polypeptide corresponding to a marker can be isolated from cells or tissue sources by an appropriate purification scheme using standard protein purification techniques. In another embodiment, polypeptides corresponding to a marker of the invention are produced by recombinant DNA techniques. Alternative to recombinant expression, a polypeptide corresponding to a marker of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly, such preparations of the protein have less than about 30%, 20%, 10%, 5% (by dry weight) of chemical precursors or compounds other than the polypeptide of interest.

Biologically active portions of a biomarker polypeptide include polypeptides comprising amino acid sequences sufficiently identical to or derived from a biomarker protein amino acid sequence described herein, but which includes fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have an amino acid sequence of a biomarker protein encoded by a nucleic acid molecule described herein. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See the National Center for Biotechnology Information (NCBI) website at ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins corresponding to a biomarker protein. As used herein, a “chimeric protein” or “fusion protein” comprises all or part (preferably a biologically active part) of a polypeptide corresponding to a marker of the invention operably linked to a heterologous polypeptide (i.e., a polypeptide other than the polypeptide corresponding to the marker). Within the fusion protein, the term “operably linked” is intended to indicate that the polypeptide of the invention and the heterologous polypeptide are fused in-frame to each other. The heterologous polypeptide can be fused to the amino-terminus or the carboxyl-terminus of the polypeptide of the invention.

One useful fusion protein is a GST fusion protein in which a polypeptide corresponding to a marker of the invention is fused to the carboxyl terminus of GST sequences. Such fusion proteins can facilitate the purification of a recombinant polypeptide of the invention.

In another embodiment, the fusion protein contains a heterologous signal sequence, immunoglobulin fusion protein, toxin, or other useful protein sequence. Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the biomarker polypeptides described herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a biomarker protein which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

The production and use of biomarker nucleic acid and/or biomarker polypeptide molecules described herein can be facilitated by using standard recombinant techniques. In some embodiments, such techniques use vectors, preferably expression vectors, containing a nucleic acid encoding a biomarker polypeptide or a portion of such a polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein.

The recombinant expression vectors for use in the invention can be designed for expression of a polypeptide corresponding to a marker of the invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol. 185, Academic Press, San Diego, Calif., 1991). Target biomarker nucleic acid expression from the pTrc vector relies on host RNA polymerase transcription from a hybrid trp-lac fusion promoter. Target biomarker nucleic acid expression from the pET 11d vector relies on transcription from a T7 gn10-lac fusion promoter mediated by a co-expressed viral RNA polymerase (T7 gni). This viral polymerase is supplied by host strains BL21 (DE3) or HMS174(DE3) from a resident prophage harboring a T7 gni gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the present invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The present invention further provides a recombinant expression vector comprising a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al., 1986, Trends in Genetics, Vol. 1(1)).

Another aspect of the present invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

### V. Analyzing Biomarker Nucleic Acids, Polypeptides, and Cells

Biomarker nucleic acids and/or biomarker polypeptides can be analyzed according to the methods described herein and techniques known to the skilled artisan to identify such genetic or expression alterations useful for the present invention including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

a. Methods for Detection of Copy Number and/or Genomic Nucleic Acid Mutations

Methods of evaluating the copy number and/or genomic nucleic acid status (e.g., mutations) of a biomarker nucleic acid are well-known to those of skill in the art. The presence or absence of chromosomal gain or loss can be evaluated simply by a determination of copy number of the regions or markers identified herein.

In one embodiment, a biological sample is tested for the presence of copy number changes in genomic loci containing the genomic marker.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region.

Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207-211, or of Kallioniemi (1992) Proc. Natl Acad Sci USA 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR)). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) and major copy proportion (MCP) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008)MBC Bioinform. 9, 204-219) may also be used to identify regions of amplification or deletion.

b. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Biomarker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) PNAS 86, 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos. 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression

The activity or level of a biomarker protein can be detected and/or quantified by detecting or quantifying the expressed polypeptide. The polypeptide can be detected and quantified by any of a number of means well-known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to an anti-cancer therapy (e.g., CDK4 and/or CDK6 inhibitor therapy). Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabelled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker proteinantibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Anti-biomarker protein antibodies, such as intrabodies, may also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MM. For X-radiographic techniques, suitable markers include any radioisotope that emits detectable radiation but that is not overtly harmful to the subject, such as barium or cesium, for example. Suitable markers for NMR and MM generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody, whether natural or synthetic, full length or a fragment thereof, monoclonal or polyclonal, that binds sufficiently strongly and specifically to the biomarker protein to be detected. An antibody may have a K_{d} of at most about 10⁻⁶ M, 10⁻⁷ M, 10⁻⁸ M, 10⁻⁹M, 10⁻¹⁰ M, 10⁻¹¹ M, or 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration in a biomarker nucleic acid and/or biomarker polypeptide molecule in order to, for example, identify sequences or agents that affect translation of iron-sulfur cluster biosynthesis-related genes.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

e, Methods for Detection of Cell Biomarkers

Cells can be analyzed according to well-known methods in the art. For example, in one embodiment, fluorescence activated cell sorting (FACS), also referred to as flow cytometry, is used to sort and analyze the different cell populations. Cells having a cellular marker or other specific marker of interest are tagged with an antibody, or typically a mixture of antibodies, that bind the cellular markers. Each antibody directed to a different marker is conjugated to a detectable molecule, particularly a fluorescent dye that may be distinguished from other fluorescent dyes coupled to other antibodies. A stream of tagged or “stained” cells is passed through a light source that excites the fluorochrome and the emission spectrum from the cells detected to determine the presence of a particular labeled antibody. By concurrent detection of different fluorochromes, also referred to in the art as multicolor fluorescence cell sorting, cells displaying different sets of cell markers may be identified and isolated from other cells in the population. Other FACS parameters, including, by way of example and not limitation, side scatter (SSC), forward scatter (FSC), and vital dye staining (e.g., with propidium iodide) allow selection of cells based on size and viability. FACS sorting and analysis of HSC and related lineage cells is well-known in the art and described in, for example, U.S. Pat. Nos. 5,137,809; 5,750,397; 5,840,580; 6,465,249; Manz et al. (202) Proc. Natl. Acad. Sci. U.S.A. 99:11872-11877; and Akashi et al. (200) Nature 404:193-197. General guidance on fluorescence activated cell sorting is described in, for example, Shapiro (2003) Practical Flow Cytometry, 4th Ed., Wiley-Liss (2003) and Ormerod (2000) Flow Cytometry: A Practical Approach, 3rd Ed., Oxford University Press.

Another method of isolating useful cell populations involves a solid or insoluble substrate to which is bound antibodies or ligands that interact with specific cell surface markers. In immunoadsorption techniques, cells are contacted with the substrate (e.g., column of beads, flasks, magnetic particles, etc.) containing the antibodies and any unbound cells removed. Immunoadsorption techniques may be scaled up to deal directly with the large numbers of cells in a clinical harvest. Suitable substrates include, by way of example and not limitation, plastic, cellulose, dextran, polyacrylamide, agarose, and others known in the art (e.g., Pharmacia Sepharose 6 MB macrobeads). When a solid substrate comprising magnetic or paramagnetic beads is used, cells bound to the beads may be readily isolated by a magnetic separator (see, e.g., Kato and Radbruch (1993) Cytometry 14:384-92). Affinity chromatographic cell separations typically involve passing a suspension of cells over a support bearing a selective ligand immobilized to its surface. The ligand interacts with its specific target molecule on the cell and is captured on the matrix. The bound cell is released by the addition of an elution agent to the running buffer of the column and the free cell is washed through the column and harvested as a homogeneous population. As apparent to the skilled artisan, adsorption techniques are not limited to those employing specific antibodies, and may use nonspecific adsorption. For example, adsorption to silica is a simple procedure for removing phagocytes from cell preparations.

FACS and most batch wise immunoadsorption techniques may be adapted to both positive and negative selection procedures (see, e.g., U.S. Pat. No. 5,877,299). In positive selection, the desired cells are labeled with antibodies and removed away from the remaining unlabeled/unwanted cells. In negative selection, the unwanted cells are labeled and removed. Another type of negative selection that may be employed is use of antibody/complement treatment or immunotoxins to remove unwanted cells.

It is to be understood that the purification or isolation of cells also includes combinations of the methods described above. A typical combination may comprise an initial procedure that is effective in removing the bulk of unwanted cells and cellular material, for example leukopharesis. A second step may include isolation of cells expressing a marker common to one or more of the progenitor cell populations by immunoadsorption on antibodies bound to a substrate. An additional step providing higher resolution of different cell types, such as FACS sorting with antibodies to a set of specific cellular markers, may be used to obtain substantially pure populations of the desired cells.

### 3. Immunomodulatory Therapies

Immunomodulatory therapies, (e.g., at least one CDK4 and/or CDK6 inhibitor, either alone or in combination with an immunotherapy, such as an immune checkpoint inhibition therapy) for use in vitro, ex vivo, and/or in vivo in a subject are provided herein. In one embodiment, such therapy (e.g., at least one CDK4 and/or CDK6 inhibitor, either alone or in combination with an immunotherapy, such as an immune checkpoint inhibition therapy) or combinations of therapies (e.g., further comprising a vaccine, chemotherapy, radiation, epigenetic modifiers, targeted therapy, and the like) can be administered to a desired subject or once a subject is indicated as being a likely responder to therapy. In another embodiment, such therapy or therapies can be avoided once a subject is indicated as not being a likely responder to the therapy or therapies and an alternative treatment regimen can be administered.

As described further below, immune responses can be upregulated in vitro, ex vivo, and/or in vivo. An exemplary ex vivo approach, for instance, involves removing immune cells from the patient, contacting immune cells in vitro with an agent described herein, and reintroducing the in vitro modulated immune cells into the patient.

In some embodiments, particular combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with or a therapy described herein (e.g., at least one CDK4 and/or CDK6 inhibitor, either alone or in combination with an immunotherapy, such as an immune checkpoint inhibition therapy). For example, it may be desirable to further administer other agents that upregulate immune responses, for example, forms of other B7 family members that transduce signals via costimulatory receptors, in order to further augment the immune response. Such additional agents and therapies are described further below. In addition, it is to be understood that a combination having more than one agent can be administered as a combined single composition or administered separately (simultaneously and/or sequentially). For example, at least one agent can be preadministered to achieve a certain effect (e.g., increasing MHC expression, reducing Tregs, etc.) before subsequent administration of a combination of the at least one agent and one or more additional agents or therapies that upregulates an immune response.

Agents that upregulate an immune response can be used prophylactically in vaccines against various polypeptides (e.g., polypeptides derived from pathogens). Immunity against a pathogen (e.g., a virus) can be induced by vaccinating with a viral protein along with an agent that upregulates an immune response, in an appropriate adjuvant.

In another embodiment, upregulation or enhancement of an immune response function, as described herein, is useful in the induction of tumor immunity.

In another embodiment, the immune response can be stimulated by the methods described herein, such that preexisting tolerance, clonal deletion, and/or exhaustion (e.g., T cell exhaustion) is overcome. For example, immune responses against antigens to which a subject cannot mount a significant immune response, e.g., to an autologous antigen, such as a tumor specific antigens can be induced by administering appropriate agents described herein that upregulate the immune response. In one embodiment, an autologous antigen, such as a tumor-specific antigen, can be coadministered. In another embodiment, the subject agents can be used as adjuvants to boost responses to foreign antigens in the process of active immunization.

In one embodiment, immune cells are obtained from a subject and cultured ex vivo in the presence of an agent as described herein, to expand the population of immune cells and/or to enhance immune cell activation. In a further embodiment the immune cells are then administered to a subject. Immune cells can be stimulated in vitro by, for example, providing to the immune cells a primary activation signal and a costimulatory signal, as is known in the art. Various agents can also be used to costimulate proliferation of immune cells. In one embodiment immune cells are cultured ex vivo according to the method described in PCT Application No. WO 94/29436. The costimulatory polypeptide can be soluble, attached to a cell membrane, or attached to a solid surface, such as a bead.

In still another embodiment, agents described herein useful for upregulating immune responses can further be linked, or operatively attached, to toxins using techniques that are known in the art, e.g., crosslinking or via recombinant DNA techniques. Such agents can result in cellular destruction of desired cells. In one embodiment, a toxin can be conjugated to an antibody, such as a bispecific antibody. Such antibodies are useful for targeting a specific cell population, e.g., using a marker found only on a certain type of cell. The preparation of immunotoxins is, in general, well known in the art (see, e.g., U.S. Pat. No. 4,340,535, and EP 44167). Numerous types of disulfide-bond containing linkers are known which can successfully be employed to conjugate the toxin moiety with a polypeptide. In one embodiment, linkers that contain a disulfide bond that is sterically “hindered” are preferred, due to their greater stability in vivo, thus preventing release of the toxin moiety prior to binding at the site of action. A wide variety of toxins are known that may be conjugated to polypeptides or antibodies of the invention. Examples include: numerous useful plant-, fungus- or even bacteria-derived toxins, which, by way of example, include various A chain toxins, particularly ricin A chain, ribosome inactivating proteins such as saporin or gelonin, α-sarcin, aspergillin, restrictocin, ribonucleases, such as placental ribonuclease, angiogenic, diphtheria toxin, and Pseudomonas exotoxin, etc. A preferred toxin moiety for use in connection with the invention is toxin A chain which has been treated to modify or remove carbohydrate residues, deglycosylated A chain. (U.S. Pat. No. 5,776,427). Infusion of one or a combination of such cytotoxic agents, (e.g., ricin fusions) into a patient may result in the death of immune cells.

In particular, CDK4 and/or CDK6 inhibitors and exemplary agents useful for inhibiting the CDK4 and/or CDK6, or other biomarkers described herein, have been described above.

Other immunomodulatory therapies useful according to the methods of the present invention are also well-known in the art.

The term “targeted therapy” refers to administration of agents that selectively interact with a chosen biomolecule to thereby treat cancer, such as an immunotherapy. For example, bevacizumab (Avastin®) is a humanized monoclonal antibody that targets vascular endothelial growth factor (see, for example, U.S. Pat. Publ. 2013/0121999, WO 2013/083499, and Presta et al. (1997) Cancer Res. 57:4593-4599) to inhibit angiogenesis accompanying tumor growth. In some cases, targeted therapy can be a form of immunotherapy depending on whether the target regulates immunomodulatory function. In another example, targeted therapy regarding the inhibition of immune checkpoint inhibitor is useful in combination with the methods of the present invention. The term “immune checkpoint inhibitor” means a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR (see, for example, WO 2012/177624). Inhibition of one or more immune checkpoint inhibitors can block or otherwise neutralize inhibitory signaling to thereby upregulate an immune response in order to more efficaciously treat cancer.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Moreover, certain immunotherapies can be used to promote immune responses. Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In one embodiment, immunotherapy comprises adoptive cell-based immunotherapies. Well known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (MET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, patient-specific neoantigen vaccines, and the like.

In another embodiment, immunotherapy comprises non-cell-based immunotherapies. In one embodiment, compositions comprising antigens with or without vaccine-enhancing adjuvants are used. Such compositions exist in many well known forms, such as peptide compositions, oncolytic viruses, recombinant antigen comprising fusion proteins, and the like. In still another embodiment, immunomodulatory interleukins, such as IL-2, IL-6, IL-7, IL-12, IL-17, IL-23, and the like, as well as modulators thereof (e.g., blocking antibodies or more potent or longer lasting forms) are used. In yet another embodiment, immunomodulatory cytokines, such as interferons, G-CSF, imiquimod, TNFalpha, and the like, as well as modulators thereof (e.g., blocking antibodies or more potent or longer lasting forms) are used. In another embodiment, immunomodulatory chemokines, such as CCL3, CCL26, and CXCL7, and the like, as well as modulators thereof (e.g., blocking antibodies or more potent or longer lasting forms) are used. In another embodiment, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

The term “untargeted therapy” referes to administration of agents that do not selectively interact with a chosen biomolecule yet treat cancer. Representative examples of untargeted therapies include, without limitation, chemotherapy, gene therapy, and radiation therapy.

For example, nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, agents and therapies other than immunotherapy or in combination thereof can be used to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), and the like are well-known in the art.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of beta-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In still another embodiment, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C (indole-3-carbinol)/DIM (di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet another embodiment, immunomodulatory antibodies or protein are used. For example, antibodies that bind to CD40, Toll-like receptor (TLR), OX-40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early nonsmall cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The duration and/or dose of treatment with anti-cancer therapy (e.g., at least one CDK4 and/or CDK6 inhibitor, either alone or in combination with an immunotherapy, such as an immune checkpoint inhibition therapy) may vary according to the particular CDK4 and/or CDK6 inhibitor agent or combination thereof. An appropriate treatment time for a particular cancer therapeutic agent will be appreciated by the skilled artisan. The invention contemplates the continued assessment of optimal treatment schedules for each cancer therapeutic agent, where the phenotype of the cancer of the subject as determined by the methods of the invention is a factor in determining optimal treatment doses and schedules.

Any means for the introduction of a polynucleotide into mammals, human or non-human, or cells thereof may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

The nucleic acids encoding a protein or nucleic acid of interest may be in a plasmid or viral vector, or other vector as is known in the art. Such vectors are well-known and any can be selected for a particular application. In one embodiment of the invention, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide of the invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J. Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### 4. Clinical Efficacy

Clinical efficacy can be measured by any method known in the art. For example, the response to a therapy described herein (e.g., at least one CDK4 and/or CDK6 inhibitor, either alone or in combination with an immunotherapy, such as an immune checkpoint inhibition therapy), relates to an immune response, such as a response of a cancer, e.g., a tumor, to the therapy, preferably to a change in tumor mass and/or volume after initiation of neoadjuvant or adjuvant chemotherapy. For example, tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular CDK4 and/or CDK6 inhibitor therapeutic regimen is at least 25%, 30%, 35%, 40%, 45%, 50%, 55%, 60%, 65%, 70%, 75%, 80%, 85%, or more.

Additional criteria for evaluating a response to therapy (e.g., at least one CDK4 and/or CDK6 inhibitor, either alone or in combination with an immunotherapy, such as an immune checkpoint inhibition therapy) are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular CDK4 and/or CDK6 inhibitor therapeutic regimen can be administered to a population of subjects and the outcome can be correlated to biomarker measurements that were determined prior to administration of any therapy of interest (e.g., at least one CDK4 and/or CDK6 inhibitor, either alone or in combination with an immunotherapy, such as an immune checkpoint inhibition therapy). The outcome measurement may be pathologic response to therapy given in the neoadjuvant setting. Alternatively, outcome measures, such as overall survival and disease-free survival can be monitored over a period of time for subjects following therapy (e.g., at least one CDK4 and/or CDK6 inhibitor, either alone or in combination with an immunotherapy, such as an immune checkpoint inhibition therapy) for whom biomarker measurement values are known. In certain embodiments, the same doses of CDK4 and/or CDK6 inhibitor agents are administered to each subject. In related embodiments, the doses administered are standard doses known in the art for CDK4 and/or CDK6 inhibitor agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of therapy (e.g., at least one CDK4 and/or CDK6 inhibitor, either alone or in combination with an immunotherapy, such as an immune checkpoint inhibition therapy) can be determined using methods such as those described in the Examples section and description provided herein. For example, therapeutic responses in settings other than cancers, such as in infections, immune disorders, and the like, are provided herein and are useful as measures of therapeutic efficacy.

### 5. Further Uses and Methods of the Present Invention

The compositions described herein can be used in a variety of diagnostic, prognostic, and therapeutic applications regarding biomarkers described herein, such as those listed in Table 1. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods

One aspect of the present invention relates to screening assays, including non-cell based assays. In one embodiment, the assays provide a method for identifying whether a cancer is likely to respond to a therapy (e.g., at least one CDK4 and/or CDK6 inhibitor, either alone or in combination with an immunotherapy, such as an immune checkpoint inhibition therapy) and/or whether an agent can inhibit the growth of or kill a cancer cell that is unlikely to respond to the therapy (e.g., at least one CDK4 and/or CDK6 inhibitor, either alone or in combination with an immunotherapy, such as an immune checkpoint inhibition therapy).

In one embodiment, the invention relates to assays for screening test agents which bind to, or modulate the biological activity of, at least one biomarker listed in Table 1. In one embodiment, a method for identifying such an agent entails determining the ability of the agent to modulate, e.g. inhibit, the at least one biomarker listed in Table 1.

In one embodiment, an assay is a cell-free or cell-based assay, comprising contacting at least one biomarker listed in Table 1, with a test agent, and determining the ability of the test agent to modulate (e.g. inhibit) the enzymatic activity of the biomarker, such as by measuring direct binding of substrates or by measuring indirect parameters as described below.

In another embodiment, an assay is a cell-free or cell-based assay, comprising contacting at least one biomarker listed in Table 1, with a test agent, and determining the ability of the test agent to modulate the ability of the biomarker to regulate CDK4/6 and/or immue checkpoints, such as by measuring direct binding of substrates or by measuring indirect parameters as described below.

For example, in a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies of the present invention can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between the biomarker and its natural binding partner can be accomplished by determining the ability of the test agent to modulate the activity of a polypeptide or other product that functions downstream or upstream of its position within the CDK4/6.

The present invention further pertains to novel agents identified by the above-described screening assays. Accordingly, it is within the scope of this invention to further use an agent identified as described herein in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the presence, absence, amount, and/or activity level of a biomarker described herein, such as those listed in Table 1, in the context of a biological sample (e.g., blood, serum, cells, or tissue) to thereby determine whether an individual afflicted with a cancer is likely to respond to a therapy (e.g., at least one CDK4 and/or CDK6 inhibitor, either alone or in combination with an immunotherapy, such as an immune checkpoint inhibition therapy), such as in an original or recurrent cancer. Such assays can be used for prognostic or predictive purpose to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those listed in Table 1.

Another aspect of the present invention pertains to monitoring the influence of agents (e.g., drugs, compounds, and small nucleic acid-based molecules) on the expression or activity of a biomarker listed in Table 1. These and other agents are described in further detail in the following sections.

The skilled artisan will also appreciate that, in certain embodiments, the methods of the present invention implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from cancerous or pre-cancerous tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-cancerous tissue of a subject and/or profiles generated from population-based distributions of informative loci of interest in relevant populations of the same species can be stored and later compared to that of a sample derived from the cancerous tissue of the subject or tissue suspected of being cancerous of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample is associated with a cancer that is likely to respond to a therapy (e.g., at least one CDK4 and/or CDK6 inhibitor, either alone or in combination with an immunotherapy, such as an immune checkpoint inhibition therapy). In some embodiments, the present invention is useful for classifying a sample (e.g., from a subject) as associated with or at risk for responding to or not responding to a therapy (e.g., at least one CDK4 and/or CDK6 inhibitor, either alone or in combination with an immunotherapy, such as an immune checkpoint inhibition therapy) using a statistical algorithm and/or empirical data (e.g., the amount or activity of at least one biomarker listed in Table 1).

An exemplary method for detecting the amount or activity of a biomarker listed in Table 1, and thus useful for classifying whether a sample is likely or unlikely to respond to a therapy (e.g., at least one CDK4 and/or CDK6 inhibitor, either alone or in combination with an immunotherapy, such as an immune checkpoint inhibition therapy) involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely anti-cancer therapy (e.g., at least one CDK4 and/or CDK6 inhibitor, either alone or in combination with an immunotherapy, such as an immune checkpoint inhibition therapy) responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In another embodiment, the diagnosis of a subject is followed by administering to the individual a therapeutically effective amount of a defined treatment based upon the diagnosis.

In one embodiment, the methods further involve obtaining a control biological sample (e.g., biological sample from a subject who does not have a cancer or whose cancer is susceptible to a therapy (e.g., at least one CDK4 and/or CDK6 inhibitor, either alone or in combination with an immunotherapy, such as an immune checkpoint inhibition therapy), a biological sample from the subject during remission, or a biological sample from the subject during treatment for developing a cancer progressing despite therapy (e.g., at least one CDK4 and/or CDK6 inhibitor, either alone or in combination with an immunotherapy, such as an immune checkpoint inhibition therapy).

d. Prognostic Assays

The diagnostic methods described herein can furthermore be utilized to identify subjects having or at risk of developing cancer condition that is likely or unlikely to be responsive to a therapy (e.g., at least one CDK4 and/or CDK6 inhibitor, either alone or in combination with an immunotherapy, such as an immune checkpoint inhibition therapy). The assays described herein, such as the preceding diagnostic assays or the following assays, can be utilized to identify a subject having or at risk of developing a disorder associated with a misregulation of the amount or activity of at least one biomarker described in, for example, Table 1, such as in cancer. Alternatively, the prognostic assays can be utilized to identify a subject having or at risk for developing a disorder associated with a misregulation of the at least one biomarker described in Table 1, such as in cancer. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity.

e. Treatment Methods

Another aspect of the invention pertains to methods of modulating the expression or activity of one or more biomarkers described herein (e.g., those listed in Table 1, and the Examples, or fragments thereof,) for therapeutic purposes. The biomarkers of the present invention have been demonstrated to be useful for identifying immunomodulatory interventions. Accordingly, the activity and/or expression of the biomarker, as well as the interaction between one or more biomarkers or a fragment thereof and its natural binding partner(s) or a fragment(s) thereof, can be modulated in order to modulate immune responses, such as in cancer.

Modulatory methods of the invention involve contacting a cell with one or more biomarkers of the invention, including one or more biomarkers of the invention, including one or more biomarkers listed in Table 1, and the Examples, or a fragment thereof or agent that modulates one or more of the activities of biomarker activity associated with the cell. An agent that modulates biomarker activity can be an agent as described herein, such as a nucleic acid or a polypeptide, a naturally-occurring binding partner of the biomarker, an antibody against the biomarker, a combination of antibodies against the biomarker and antibodies against other immune related targets, one or more biomarkers agonist or antagonist, a peptidomimetic of one or more biomarkers agonist or antagonist, one or more biomarkers peptidomimetic, other small molecule, or small RNA directed against or a mimic of one or more biomarkers nucleic acid gene expression product.

An agent that modulates the expression of one or more biomarkers of the present invention, including one or more biomarkers of the invention, including one or more biomarkers listed in Table 1, and the Examples, or a fragment thereof is, e.g., an antisense nucleic acid molecule, RNAi molecule, shRNA, mature miRNA, pre-miRNA, pri-miRNA, miRNA*, anti-miRNA, or a miRNA binding site, or a variant thereof, or other small RNA molecule, triplex oligonucleotide, ribozyme, or recombinant vector for expression of one or more biomarkers polypeptide. For example, an oligonucleotide complementary to the area around one or more biomarkers polypeptide translation initiation site can be synthesized. One or more antisense oligonucleotides can be added to cell media, typically at 200 μg/ml, or administered to a patient to prevent the synthesis of one or more biomarkers polypeptide. The antisense oligonucleotide is taken up by cells and hybridizes to one or more biomarkers mRNA to prevent translation. Alternatively, an oligonucleotide which binds double-stranded DNA to form a triplex construct to prevent DNA unwinding and transcription can be used. As a result of either, synthesis of biomarker polypeptide is blocked. When biomarker expression is modulated, preferably, such modulation occurs by a means other than by knocking out the biomarker gene.

Agents which modulate expression, by virtue of the fact that they control the amount of biomarker in a cell, also modulate the total amount of biomarker activity in a cell.

In one embodiment, the agent stimulates one or more activities of one or more biomarkers of the invention, including one or more biomarkers listed in Table 1 and the Examples or a fragment thereof. Examples of such stimulatory agents include active biomarker polypeptide or a fragment thereof and a nucleic acid molecule encoding the biomarker or a fragment thereof that has been introduced into the cell (e.g., cDNA, mRNA, shRNAs, siRNAs, small RNAs, mature miRNA, pre-miRNA, pri-miRNA, miRNA*, anti-miRNA, or a miRNA binding site, or a variant thereof, or other functionally equivalent molecule known to a skilled artisan). In another embodiment, the agent inhibits one or more biomarker activities. In one embodiment, the agent inhibits or enhances the interaction of the biomarker with its natural binding partner(s). Examples of such inhibitory agents include antisense nucleic acid molecules, anti-biomarker antibodies, biomarker inhibitors, and compounds identified in the screening assays described herein.

These modulatory methods can be performed in vitro (e.g., by contacting the cell with the agent) or, alternatively, by contacting an agent with cells in vivo (e.g., by administering the agent to a subject). As such, the present invention provides methods of treating an individual afflicted with a condition or disorder that would benefit from up- or down-modulation of one or more biomarkers of the present invention listed in Table 1 and the Examples or a fragment thereof, e.g., a disorder characterized by unwanted, insufficient, or aberrant expression or activity of the biomarker or fragments thereof. In one embodiment, the method involves administering an agent (e.g., an agent identified by a screening assay described herein), or combination of agents that modulates (e.g., upregulates or downregulates) biomarker expression or activity. In another embodiment, the method involves administering one or more biomarkers polypeptide or nucleic acid molecule as therapy to compensate for reduced, aberrant, or unwanted biomarker expression or activity.

Stimulation of biomarker activity is desirable in situations in which the biomarker is abnormally downregulated and/or in which increased biomarker activity is likely to have a beneficial effect. Likewise, inhibition of biomarker activity is desirable in situations in which biomarker is abnormally upregulated and/or in which decreased biomarker activity is likely to have a beneficial effect.

In addition, these modulatory agents can also be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, these modulatory agents can be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and can be determined by the physician.

### 6. Pharmaceutical Compositions

In another aspect, the present invention provides pharmaceutically acceptable compositions which comprise a therapeutically-effective amount of an agent that modulates (e.g., decreases) biomarker expression and/or activity, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) biomarker expression and/or activity which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) biomarker expression and/or activity. These salts can be prepared in situ during the final isolation and purification of the respiration uncoupling agents, or by separately reacting a purified respiration uncoupling agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) biomarker expression. These salts can likewise be prepared in situ during the final isolation and purification of the respiration uncoupling agents, or by separately reacting the purified respiration uncoupling agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) biomarker expression and/or activity, with the carrier and, optionally, one or more accessory ingredients. In general, the formulations are prepared by uniformly and intimately bringing into association a respiration uncoupling agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a respiration uncoupling agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more respiration uncoupling agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) biomarker expression and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a respiration uncoupling agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) biomarker expression and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) biomarker expression and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a respiration uncoupling agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more respiration uncoupling agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) biomarker expression and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the respiration uncoupling agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The present invention also encompasses kits for detecting and/or modulating biomarkers described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

Other embodiments of the present invention are described in the following Examples. The present invention is further illustrated by the following examples which should not be construed as further limiting.

### EXAMPLES

### Example 1: Materials and Methods for Examples 2-8

a. Animal Experiments

Tumor formation was induced in MMTV-rtTA/tetO-HER2 mice with doxycycline as previously described in Goel et al. (2016) Cancer Cell 29:255-269. Female FVB mice (7 weeks of age) were purchased from Taconic Biosciences (Hudson, N.Y.). Female J:NU nude mice (8 weeks of age) were purchased from Jackson Labs (Bar Harbor, Me.). FVB CD45.2+ mice used as a source of T cells for in vitro studies were a kind gift from Dr. Daniel Tenen. For tumor growth studies in J:NU mice, MMTV-rtTA/tetO-HER2 tumor explants were orthotopically implanted bilaterally into nude mice, as previously described in Goel et al. (2016), supra. For tumor growth experiments in J:NU mice, treatment with abemaciclib or vehicle was begun when tumors reached 5-10 mm diameter, and mice were randomized into treatment groups such that distribution of tumor volumes was even between groups. For tumor experiments in transgenic MMTV-rtTA/tetO-HER2 mice, tumors measured between 5 mm and 15 mm when treatment was begun. Mice were assigned into treatment groups so that the distribution of tumor volumes was even between groups. Tumor volume was calculated as previously described in Goel et al. (2016), supra and tumors were measured by caliper 2-3 times per week. For tumor growth curve analysis, one-way ANOVA tests were performed with correction for multiple comparisons using Sidak's multiple comparisons test.

Abemaciclib (75-90 mg/kg, as noted for individual experiments (Haoyuan Chemexpress, Shanghai, China), prepared as previously described in Goel et al. (2016), supra) and palbociclib (90 mg/kg (Haoyuan Chemexpress), diluted in 50 nM sodium d-lactate), were administered by oral gavage daily. For CD8 depletion experiments, tumor-bearing mice were treated with anti-CD8 antibody (400 μg by intraperitoneal injection; BioXcell (West Lebanon, N.H.), clone YTS 169.4) or isotype control (400 μg by intraperitoneal injection; clone LTF-2, BioXcell) 48 and 24 hours prior to beginning abemaciclib therapy (90 mg/kg) and every 5 days thereafter. For combination therapy experiments with anti-PDL1 therapy, treatment with abemaciclib was initiated at 90 mg/kg daily. After 3 days, the dose was dropped to 75 mg/kg daily and treatment with an anti-PD-L1 antibody (200 μg by intraperitoneal injection every 72 hours; BioXcell, clone 10F.9G2) or an isotype control antibody (200 μg by intraperitoneal injection every 72 hours) was commenced. Mice were euthanized using CO₂ inhalation, and all mouse experiments were performed in accordance with protocols approved by the Institutional Animal Care and Use Committees of Dana-Farber Cancer Institute, Harvard Medical School, and Boston Children's Hospital.

b. Immunohistochemistry and Immunofluorescence

Immunostaining for Ki-67, HER2, and STAT1 were performed as previously described in Goel et al. (2016), supra. The anti-Ki-67 antibody was obtained from Vector (Burlingame, Calif.) and the anti-HER2 and anti-STAT1 antibodies were obtained from Abcam (Cambridge, UK). Secondary antibodies (AF488 AffiniPure donkey anti-mouse IgG and Cy3 AffiniPure donkey anti-rabbit IgG) were purchased from Jackson ImmunoResearch Laboratories (West Grove, Pa.). Images were acquired with a Yokogawa spinning disk confocal on an inverted Nikon Ti fluorescence microscope using MetaMorph® image acquisition software, and 3-5 fields were analyzed per tumor. Image analysis was performed using a semi-automated in-house platform (NIH ImageJ).

FoxP3 immunofluorescence was performed as previously described in McAllister et al. (2008) Cell 133:994-1005. CD3 (clone SP7) was purchased from Abcam and FoxP3 (clone FJK-16s) was purchased from eBioscience, and Ki-67 antibody (clone SP6) was purchased from Thermo Scientific (Waltham, Mass.). Secondary antibodies (AF488 donkey anti-rabbit IgG, AF647 goat anti-rat IgG) were purchased from Life Technologies. Tissues were counterstained with DAPI (Invitrogen). Images were acquired on a Nikon Eclipse Ni microscope using NIS Elements software, and 5-10 fields were analyzed per tumor.

c. Cell Lines

BT474, SKBR3, MDA-MB-361, and MDA-MB-453 human breast cancer cell lines were maintained as previously described in Goel et al. (2016), supra with slight modifications: antibiotics and anti-fungals were not used in cell culture media. All cell lines were obtained from ATCC, tested negative for mycoplasma, and their identity was verified by short tandem repeat analysis (Promega GenePrint® 10 System).

d. In Vitro Drug Studies

Lapatinib was purchased from Haoyuan Chemexpress. Abemaciclib and lapatinib were diluted as previously described in Goel et al. (2016), supra. For in vitro use, palbociclib was diluted in DMSO. Cleaved PARP was measured after 48 hours of treatment with DMSO, lapatinib, or abemaciclib. Lapatinib dosages: 30 nM for BT474 and SKBR3, and 500 nM for MDA-MB-453 and MDA-MB-361. abemaciclib dosages: 300 nM for BT474 and SKBR3; 25 nM for MDA-MB-453; 500 nM for MDA-MB-361. For staurosporine experiments, MDA-MB-453 cells were pretreated with DMSO or abemaciclib (500 nM) for 0, 1, or 7 days prior to exposure to staurosporine (500 nM, Enzo Life Sciences, Farmingdale, N.Y.) for 4 hours. To determine JAK dependency of effects, cells were treated with appropriate combinations of DMSO, abemaciclib (500 nM), and ruxolitinib (500 nM, Selleckchem, Houston, Tex.) for 7 days.

e. Western Blots

Western blotting was carried out as previously described in Goel et al. (2016), supra. Antibodies to cleaved PARP, cleaved caspase-3, phospho-STAT1 Y701, STAT1, and FLAG were purchased from Cell Signaling Technologies (Danvers, Mass.), and the anti-vinculin antibody was purchased from Sigma (St. Louis, Mo.).

f. β-Galactosidase Activity

SA-beta galactosidase expression was determined as previously described in Goel et al. (2016), supra, using a senescence detection kit (Abcam, ab65351).

g. Transcriptome Methodology

AmpliSeg™ libraries were constructed and sequenced on the Ion Torrent Proton platform (Thermo Fisher) according to the manufacturer's instructions as previously described in Ni et al. (2016) Nat Med 22:723-726 and Wang et al. (2015) Cell 163:174-186. For human gene analysis in cell lines, the Ion AmpliSeg™ Transcriptome Human Gene Expression Kit is designed for targeted amplification of over 20,000 human RefSeq genes simultaneously in a single primer pool. A short amplicon (˜110 bp) is amplified for each targeted gene. Since an AmpliSeq transcriptome mouse kit is not commercially available, an Ion AmpliSeg™ Custom Panel was designed by the manufacturer (Thermo Fisher) using Ion AmpliSeg™ Designer for targeted amplification of 3,826 mouse genes that are most relevant for our studies (one short amplicon for each gene) in one primer pool for mouse studies. For each sample, 10 ng of total RNA was used for cDNA library preparation. Multiple libraries were multiplexed and clonally amplified using the Ion OneTouch™ 2 System (Thermo Fisher), and then sequenced on an Ion Torrent™ Proton machine (Thermo Fisher). Data were first analyzed by Torrent Suite and ampliSeqRNA analysis plugin (Thermo Fisher) to generate count data.

h. Mouse Transcriptome Analysis

Differential expression analysis was performed using DESeq2 (Love et al. (2014) Genome Biol 15:550) using raw read counts per gene from transcriptomic analyses. Genes deemed to be up- or down-regulated, as determined using p-value cutoff of 0.05 after Benjamini-Hochberg correction for multiple testing, were used as input as separate lists for gene ontology enrichment analysis (The Gene Ontology Consortium (2015) Gene Ontology Consortium: going forward. Nucleic Acids Res 43:D1049-D1056). Gene set enrichment analysis (Subramanian et al. (2005) Proc Natl Acad Sci USA 102:15545-15550; Mootha et al. (2003) Nat Genet 34:267-273) was carried out using normalized read counts per gene from transcriptomic analyses.

i. Human Transcriptome Analysis

Transcriptomic data from human cell lines were obtained as above, and analyses were performed on normalized read counts per gene. All experiments were performed in triplicate. Only genes with an absolute read count of >20 on at least one sample were included. The fold change in normalized read count of the included genes was determined for each replicate experiment. The mean fold change was then calculated for each gene. Genes with a mean fold change of >2 (DMSO vs abemaciclib) were then included for analysis using gene ontology enrichment analysis.

For mouse and human transcriptomic data, significant differences in gene expression were determined by t-tests, adjusted for multiple comparisons with the Benjamini-Hochberg correction.

j. TCGA Analysis

Gene expression data from The Cancer Genome Atlas was obtained using the cBioPortal for Cancer Genomics (with the World Wide Web address of cbioportal.org). Data were obtained from the breast cancer datasets “TCGA, Provisional (1105 samples)” for comparisons between cyclin D1 amplified and diploid tumors (Gao et al. (2013) Sci Signal 6:01; Cerami et al. (2012) Cancer Discov 2:401-404).

k. RT-qPCR

RT-qPCR was performed as previously described in Goel et al. (2016), supra. Primer sequences used for qPCR were as follows: Ifng (mouse) forward: 5′-ATG AAC GCT ACA CAC TGC ATC-3′ (SEQ ID NO: 10); reverse: 5′-CCA TCC TTT TGC CAG TTC CTC-3′ (SEQ ID NO: 11). Tap1 (mouse) forward: 5′-GGA CTT GCC TTG TTC CGA GAG-3′ (SEQ ID NO: 12); reverse: 5′-GCT GCC ACA TAA CTG ATA GCG A-3′ (SEQ ID NO: 13). Tap2 (mouse) forward: 5′-CTG GCG GAC ATG GCT TTA CTT-3′ (SEQ ID NO: 14); reverse: 5′-CTC CCA CTT TTA GCA GTC CCC-3′ (SEQ ID NO: 15). Tapbp (mouse) forward: 5′-GGC CTG TCT AAG AAA CCT GCC-3′ (SEQ ID NO: 16); reverse: CCA CCT TGA AGT ATA GCT TTG GG-3′ (SEQ ID NO: 17). Erap1 (mouse) forward: 5′-TAA TGG AGA CTC ATT CCC TTG GA-3′ (SEQ ID NO: 18); reverse 5′-AAA GTC AGA GTG CTG AGG TTT G-3′ (SEQ ID NO: 19). Nlrc5 (mouse) forward: 5′-GCT GAG AGC ATC CGA CTG AAC-3′ (SEQ ID NO: 20); reverse: 5′-AGG TAC ATC AAG CTC GAA GCA-3′ (SEQ ID NO: 21). Il6 (mouse) forward: 5′-TAG TCC TTC CTA CCC CAA TTT CC-3′ (SEQ ID NO: 22); reverse: 5′-TTG GTC CTT AGC CAC TCC TTC-3′ (SEQ ID NO: 23). B2M (human) forward: 5′-GAG GCT ATC CAG CGT ACT CCA-3′ (SEQ ID NO: 24); reverse: 5′-CGG CAG GCA TAC TCA TCT TTT-3′ (SEQ ID NO: 25). HLA-A (human) forward: 5′-ACC CTC GTC CTG CTA CTC TC-3′ (SEQ ID NO: 26); reverse: 5′-CTG TCT CCT CGT CCC AAT ACT-3′ (SEQ ID NO: 27). HLA-B (human) forward: 5′-CAG TTC GTG AGG TTC GAC AG-3′ (SEQ ID NO: 28); reverse: 5′-CAG CCG TAC ATG CTC TGG A-3′ (SEQ ID NO: 29). HLA-C (human) forward: 5′-GGA CAA GAG CAG AGA TAC ACG-3′ (SEQ ID NO: 30); reverse: 5′-CAA GGA CAG CTA GGA CAA CC-3′ (SEQ ID NO: 31). STAT1 (human) forward: 5′-CAG CTT GAC TCA AAA TTC CTG GA-3′ (SEQ ID NO: 32); reverse 5′-TGA AGA TTA CGC TTG CTT TTC CT-3′ (SEQ ID NO: 33). IL6 (human) forward: 5′-ACT CAC CTC TTC AGA ACG AAT TG-3′ (SEQ ID NO: 34); reverse: 5′-CCA TCT TTG GAA GGT TCA GGT TG-3′ (SEQ ID NO: 35). Relative copy number for each sample was determined by calculating the fold change difference in the gene of interest relative to GAPDH or HSP90AB1 (human) or Actb (mouse). qPCR was performed on the Applied Biosystems 7300 machine.

1. Flow cytometry

Tumor Cell Lines—

Cells were counted after trypsinization and 1 million cells per condition were stained with the appropriate antibodies diluted in PBS (Hyclone) plus 2% FBS (Life Technologies) for 30 minutes on ice. Matched fluorescence minus one (FMO) staining for each condition was performed as a control.

Blood—

Blood was obtained by retro-orbital sampling at intermediate time points during experiments and by cardiac puncture at experimental endpoints. Blood cells and plasma were separated by centrifugation at 1,500×g for 8 minutes at 4° C. Following RBC lysis (PharmLyse, BD Biosciences), blood cells were blocked with anti-CD16/32 (Biolegend, San Diego, Calif.) for 20 minutes on ice. Cells were incubated with appropriate antibodies for 30 min on ice.

Spleen and Thymus—

Following mechanical digestion and RBC lysis, single cell suspensions were blocked with anti-CD16/32 for 20 minutes on ice. Cells were incubated with appropriate antibodies for 30 minutes on ice.

Lymph Nodes—

Following mechanical digestion, single cell suspensions were blocked with anti-CD16/32 for 20 minutes on ice. Cells were incubated with appropriate antibodies for 30 minutes on ice.

Tumor—

Tumors were first mechanically disrupted by chopping, then chemically digested in dissociation buffer (2 mg/mL collagenase type IV (Worthington Biochemical, Lakewood, N.J.), 0.02 mg/mL DNase (Sigma Aldrich) in DMEM (Life Technologies) containing 5% FBS (Life Technologies), PenStrep (Hyclone)) with agitation at 37° C. for 45 minutes. After RBC lysis, single cell suspensions were blocked with anti-CD16/32 for 20 minutes on ice. Then, cells were incubated with the appropriate antibodies for 30 minutes on ice.

Murine antibodies used for flow cytometry include those recognizing CD45 (clone 30-F11), CD3 (clone 145-2C11), CD8 (clone 53-6.7), CD4 (clone RM4-5), PD-1 (clone 29F.1A12), Tim-3 (clone RMT3-23), CTLA-4 (clone UC10-4B9), LAG-3 (clone C9B7W), B220 (clone RA3-6B2), NK1.1 (clone PK136), CD11b (clone M1/70), Ly6G (clone 1A8), Ly6C (clone AL-21), FoxP3 (clone FJK-16s), and Ki-67 (clone 16A8). Human antibodies used for flow cytometry were those recognizing β2-microglobulin (clone 2M2) and HLA-A, B, C (clone W6/32). Antibodies were purchased from Biolegend with the exception of anti-FoxP3 from eBioscience and anti-Ly6C from BD Pharmigen. All antibodies used for flow cytometry were directly conjugated to fluorophores. The anti-mouse/rat FoxP3 staining set (eBioscience) was used for intracellular staining according to the manufacturer's instructions. 7AAD was used to distinguish live/dead cells, except for analyses requiring intracellular staining when eFluor® 450 fixable viability dye (eBioscience) or Zombie Yellow™ fixable viability dye (Biolegend) were used. To determine the absolute number of cells in a sample, CountBright™ absolute counting beads (Molecular Probes, ThermoFisher) were added. Flow cytometry was performed on a LSRII (BD Biosciences, San Jose, Calif.) or FACSCANTOII (BD Biosciences), and data was analyzed using FlowJo® (TreeStar).

m. CD8 T Cell Cytotoxicity Assay

Primary tumor cells were isolated as previously described in Goel et al. (2016), supra and treated in culture with DMSO or abemaciclib (500 nM) for 7 days. CD8+ T cells were isolated from the spleen and lymph nodes of an MMTV-rtTA/tetO-HER2 tumor-bearing mouse by positive selection using the MACS CD8a microbead kit (Miltenyi Biotec, Cambridge, Mass.) using an autoMACS® Pro separator. Tumor cells were labeled with CFSE (Biolegend), and 1×10⁴ tumor cells were co-cultured with CD8+ T cells at the indicated ratios for 4 hours at 37° C. Live CFSE+ tumor cells were quantified by flow cytometry, and percent survival calculated relative to that of tumor cells cultured in the absence of CD8+ T cells.

n. p16 Overexpression

p16 was expressed in MDA-MB-453 and BT464 cells by transient transfection of pBabepuro3-p16Flag (Addgene, Cambridge, Mass., Cat #24934) using Lipofectamine™ 3000 (Thermo Fisher) according to the manufacturer's instructions. Seventy-two hours after transfection cells were selected in puromycin for 48 hours. p16 overexpression was confirmed by anti-FLAG Western blots.

o. ELISAs

Cells were treated with DMSO or abemaciclib (500 nM) for 7 days. For the last 24 hours, the culture media was replaced with serum free media. Following concentration of conditioned media using Amicon® Ultra centrifugal filters (Millipore, Billerica, Mass.), cytokines were analyzed according to the manufacturer's recommendations using the following kits: human IFN gamma ELISA Ready-SET-Go!® (Affymetrix eBioscience), human TNF alpha ELISA Ready-SET-Go!® (Affymetrix eBioscience), Verikine human IFN alpha ELISA kit (PBL assay science, Piscataway, N.J.), VeriKine-HS human IFN beta serum ELISA kit (PBL assay science), human IL-28B quantikine ELISA kit (R&D Systems, Minneapolis, Minn.), human IL-28A DuoSet ELISA (R&D Systems), and human IL-29 DuoSet ELISA (R&D Systems). Anti-ANA and anti-dsDNA ELISAs (Alpha Diagnostic, San Antonio, Tex.) were performed according to the manufacturer's instructions on plasma isolated from tumor free or tumor-bearing mice. Absorbance was measured on a Synergy™ Neo plate reader (BioTek, Winooski, Vt.) using Gen5™ software.

p. Interferon Neutralization Experiments

For all neutralization experiments, cell lines were treated with DMSO or abemaciclib (500 nM) for 7 days. Neutralizing antibodies were applied for the entire duration of drug treatment, and included IFN-γ neutralizing antibody (1 μg/mL, R&D Systems) and IFN-α neutralizing antibody (2.0-2.5 μg/mL, R&D Systems). Recombinant human IFN-γ (Peprotech, Rocky Hill, N.J.; 250 pg/mL) and IFN-α (Life Technologies, Carlsbad, Calif.; 250 pg/mL) were used to determine successful neutralization by these antibodies, and were administered 24 hours prior to protein collection.

q. Doxorubicin-Induced Senescence

MDA-MB-453 and BT474 cells were treated with doxorubicin (Sigma Aldrich, 200 nM) for a period of 24 hrs. The cells were then cultured in fresh media for 72 hours after treatment, and RNA was extracted for qPCR.

r. In Vitro Regulatory T Cell Differentiation

CD4+CD25− T cells from spleens and lymph nodes of naïve FVB mice were isolated by the CD4+CD25+ Regulatory T cell kit (Miltenyl Biotec) using an autoMACs® Pro separator and cultured for 72 hours in T cell media (RPMI with 10% FBS and β-mercaptoethanol (55 nM, Life Technologies) with CD3/CD28 Dynabeads (1:1 ratio of cells:beads, ThermoFisher), 100 U/mL rhIL-2 (Peprotech), +/−25 ng/mL rhTGF-β (R&D Systems) with DMSO or abemaciclib (125-1000 nM). Percent differentiation for each condition was determined by intracellular flow cytometry for FoxP3. The fold change in percent differentiation with the addition of rhTGF-β was calculated for each condition. All fold changes in percent differentiation were normalized to the DMSO control.

s. T Cell Proliferation In Vitro

CD4+CD25- and CD4+CD25+ T cells from spleens and lymph nodes from naïve FVB mice were isolated by the CD4+CD25+ Regulatory T cell kit (Miltenyl Biotec); CD8+ T cells were isolated by the CD8a+ T cell isolation kit (Miltenyl Biotec). Isolated T cells were resuspended in RPMI (ATCC) with 5% FBS, labeled with 5 μM CFSE (Biolegend) for 10 minutes in the dark at room temperature, and washed twice in 10× volume of PBS (Hyclone) with 5% FBS. 1×10⁵ cells were cultured in T cell media with CD3/CD28 beads (1:1 ratio of cells to beads), 100 U/mL rhIL-2 and DMSO or abemaciclib (250 or 500 nM) for 72 hours at 37° C. CFSE dilution was analyzed at endpoint by flow cytometry.

t. Statistical Analyses

Statistical analyses were performed as described in the figure legend for each experiment. All statistical tests were two-sided. All data are presented as mean±SD. Differences were considered statistically significant at a p value of less than or equal to 0.05.

### Example 2: CDK4/6 Inhibition Triggers Immunologic Clearance of Breast Cancers

Pharmacologic inhibitors of cyclin-dependent kinases 4 and 6 (CDK4/6) have shown significant activity against various solid tumors. Although CDK4/6 inhibitors chiefly induce cell cycle arrest but not apoptosis, tumor regressions are seen in a subset of patients. In the Examples provided herein, murine models of breast cancer were used to show that selective CDK4/6 inhibitors, such as those currently in clinical developments, cause tumor regression by promoting anti-tumor immune responses. This anti-tumor immunity occurs through without limitation, at least two mechanisms: (i) Rb/E2F-mediated suppression of tumor cell DNA methyltransferase 1 expression, which triggers interferon-sensitive gene expressions that result in enhanced antigen presentation, and (ii) inhibition of regulatory T cell proliferation, which is caused by suppression of DNA methyltransferase 1 expression in Tregs and a consequent inhibition of their proliferation. Collectively, these effects promote cytotoxic T cell-mediated clearance of tumor cells, which can be further enhanced by the addition of immune checkpoint blockade. The results described herein indicate that CDK4/6 inhibitors increase tumor immunogenicity and suggest new combination regimens comprising CDK4/6 inhibitors and immunotherapies as anti-cancer treatment.

### Example 3: CDK4/6 Inhibitors Enhance Antigen Presentation

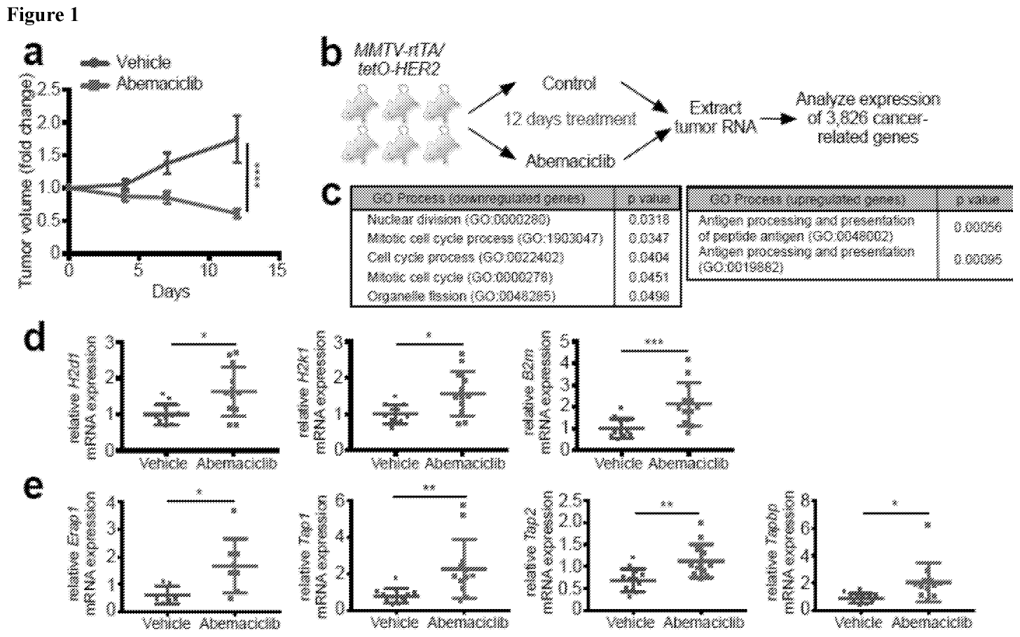

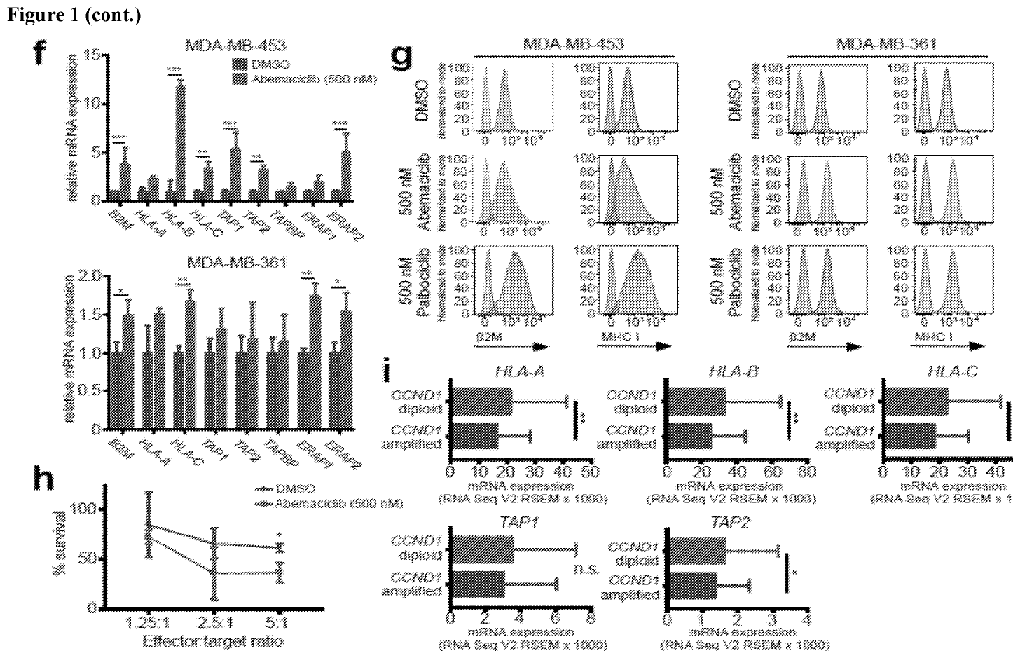

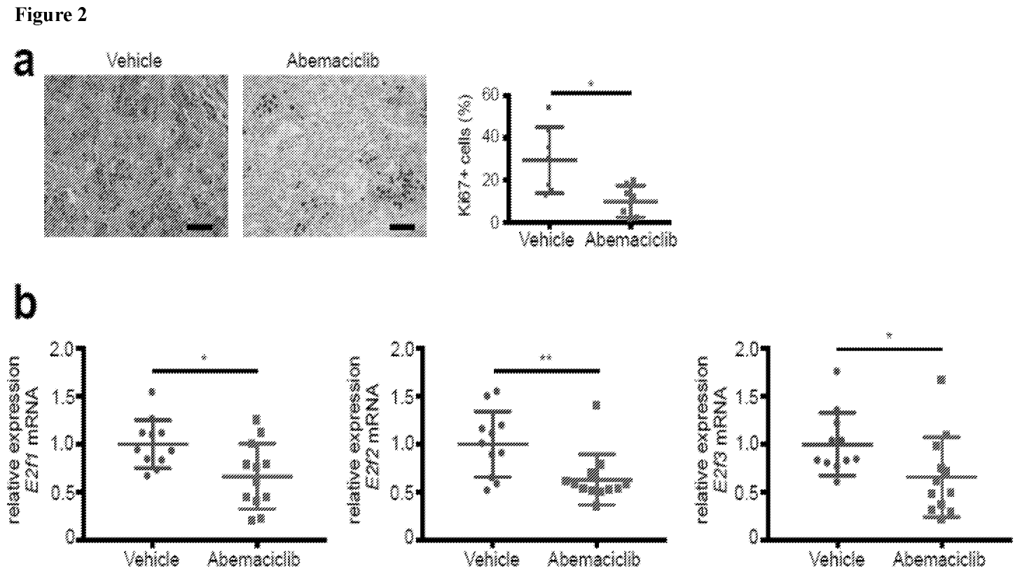

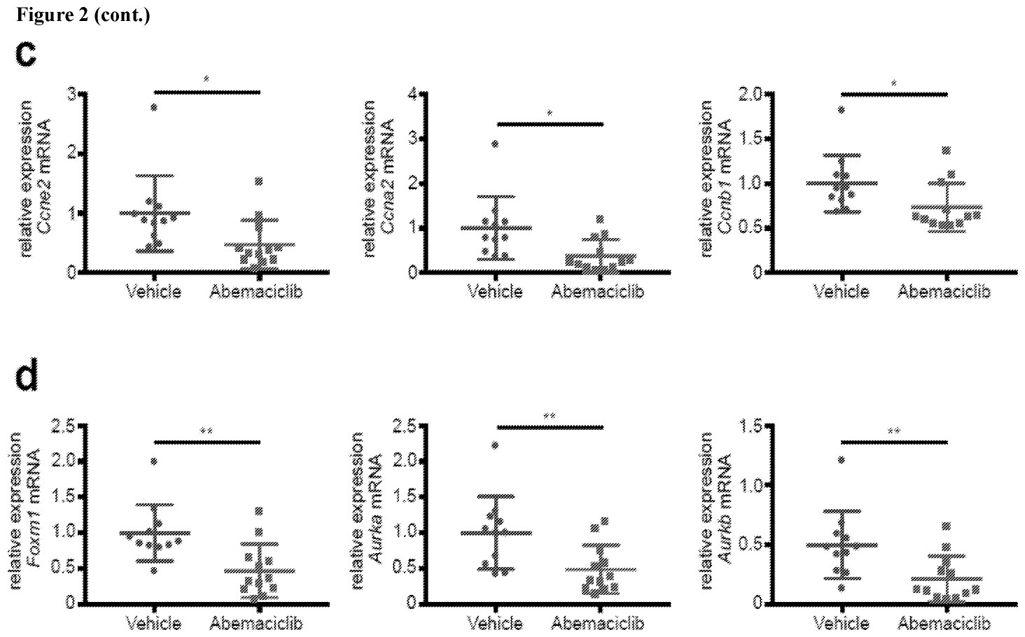

The in vivo impact of CDK4/6 inhibition was tested using the MMTV-rtTA/tetO-HER2 transgenic mouse model of mammary carcinoma, as described in Goel et al. (2016), supra. Administration of doxycycline to adult female MMTV-rtTA/tetO-HER2 mice results in mammary-specific expression of the human ERBB2 oncogene and development of mammary carcinomas with 100 percent penetrance. Importantly, cells derived from MMTV-rtTA/tetO-HER2 tumors retain Rb expression and undergo cell cycle arrest in response to CDK4/6 inhibition (Goel et al. (2016), supra). In each of three independent experiments, abemaciclib caused regression of bulky tumors that were growing prior to initiation of treatment, evidenced by an average 40 percent reduction in tumor volume at the 12-day end point (FIG. 1A). In the treated tumor, there was a significant reduction in tumor cell proliferation (FIG. 2A) and gene expression of E2F transcription factors as well as S phase- and G2/M-related genes (FIGS. 2B-2D).

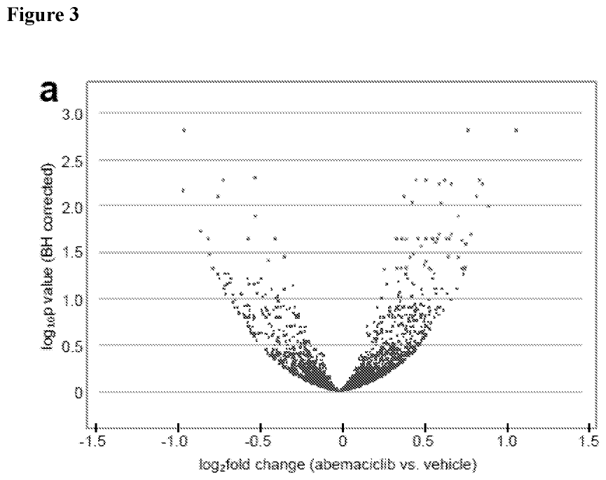

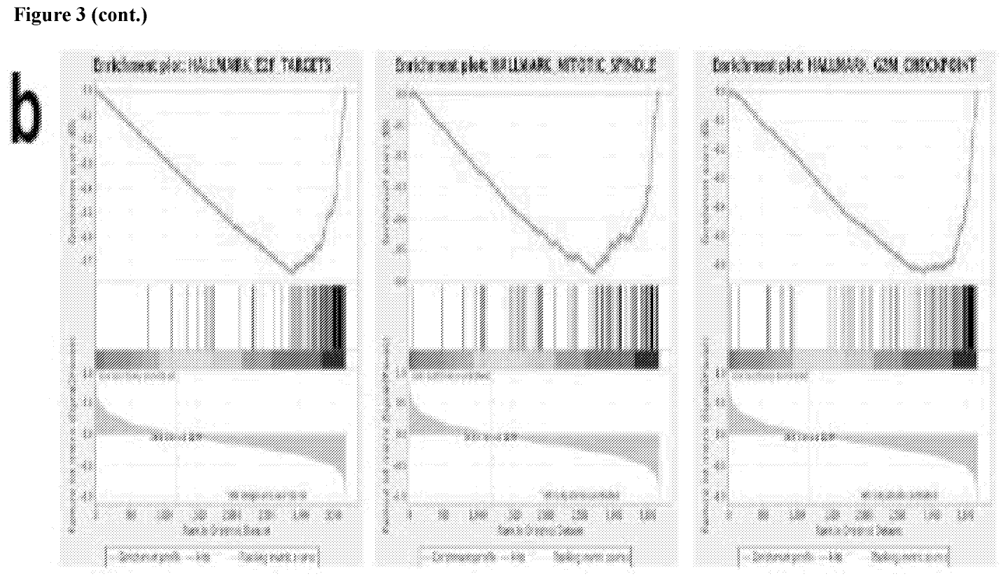

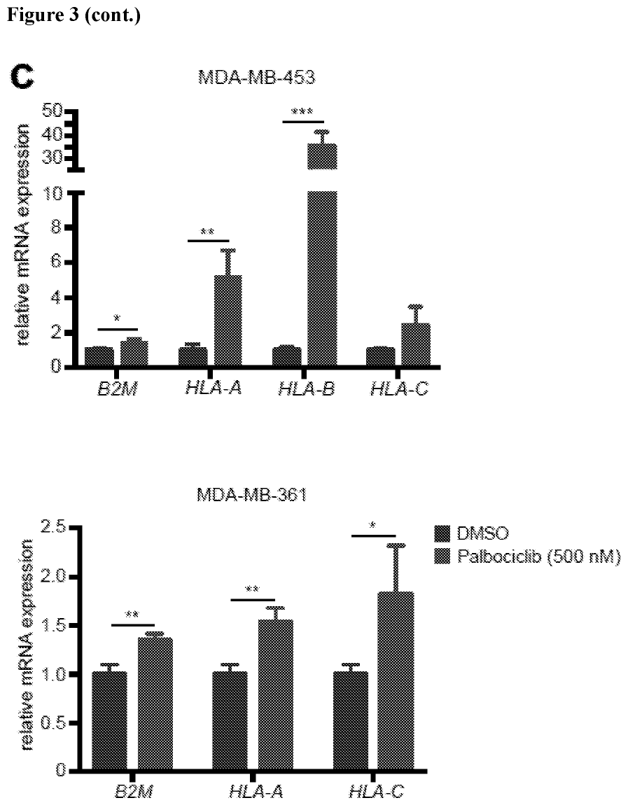

The expression profile of a panel of 3,826 cancer-related genes was measured in MMTV-rtTA/tetO-HER2 tumor tissue after 12 days of abemaciclib or vehicle treatment (FIG. 1B). The transcriptomic profiles were compared using both Gene Ontology (GO) and Gene Set Enrichment Analysis (GSEA). As result, genes significantly downregulated by abemaciclib were enriched within GO and GSEA terms relating to cell cycle, mitosis, and E2F targets (FIGS. 1C and 3A-3B). Strikingly, only two GO process terms were significantly enriched for genes upregulated by abemaciclib: “antigen processing and presentation of peptide antigen” and “antigen processing and presentation” (FIG. 1C). Specifically, genes encoding components of the murine major histocompatibility complex (MEW) class I molecule were upregulated in the abemaciclib-treated tumors (e.g., MHC class 1 genes H2d1 and H2k1, and beta-2 microglobulin B2m) (FIG. 1D). Likewise, genes directing peptide cleavage (e.g., the aminopeptidase Erap1), peptide transport (e.g., transporters associated with antigen processing, Tap1 and Tap2), and transporter-MHC interactions (e.g., tapasin, Tapbp) were also significantly upregulated by abemaciclib (FIG. 1E).

To determine if CDK4/6 inhibition directly induces expression of antigenic peptide processing and presentation genes by tumor cells, two breast cancer cell lines (MDA-MB-453 and MDA-MB-361) were treated in vitro with DMSO or abemaciclib for 7 days and measured the expression of B2M, HLA-A, HLA-B, HLA-C, TAP1, TAP2, TAPBP, ERAP1, and ERAP2. Consistent with the in vivo results, all of these genes were upregulated in both cell lines, apart from HLA-B, which was not expressed in MDA-MB-361 cells (FIG. 1F). Palbociclib treatment yielded similar results (FIG. 3C). Moreover, treatment for 7 days with either inhibitor increased tumor cell-surface expression of B2M and MHC class I proteins (FIG. 1G). To explore the functional consequences of these findings, primary MMTV-rtTA/tetO-HER2 tumor cells were treated ex vivo for 7 days with DMSO or abemaciclib. Abemaciclib-treated cells showed greater susceptibility to killing by CD8+ T cells derived from MMTV-rtTA/tetO-HER2 mice (FIG. 1H).

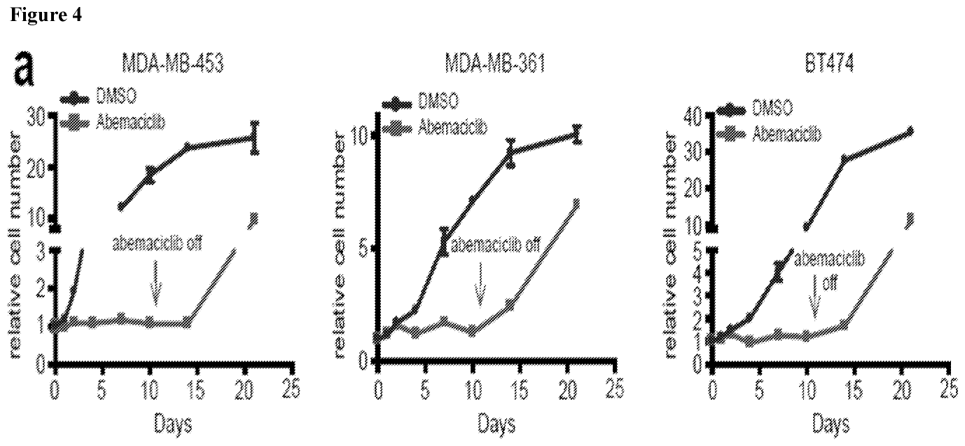

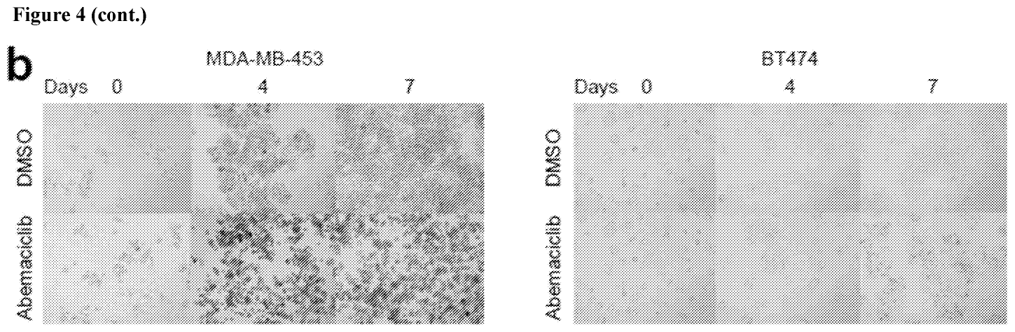

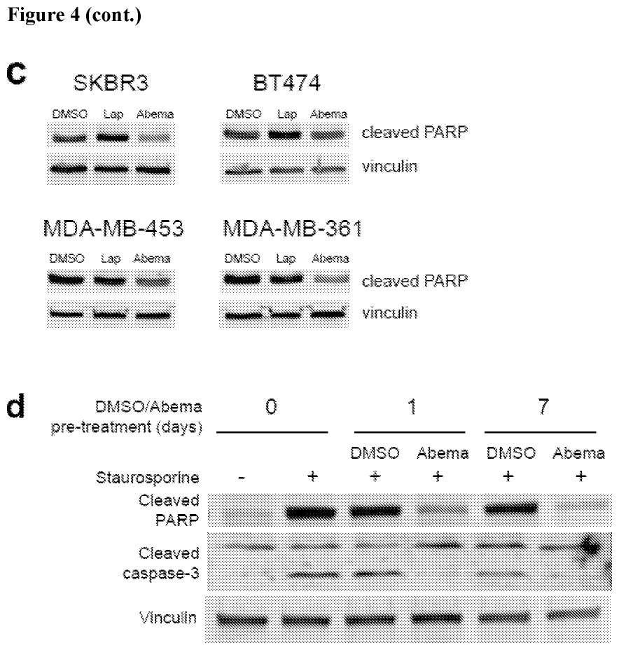

The direct effects of abemaciclib on tumor cell apoptosis were tested to determine whether this might contribute to the observed tumor regression. Three breast cancer cell lines (MDA-MB-453, MDA-MB-361, and BT474) were treated in vitro with DMSO or abemaciclib for a period of 11 days (FIG. 4A). Cell proliferation was completely suppressed during this period. However, cell number was maintained and cells did not exhibit morphologic features of apoptosis (FIGS. 4A-4B). Cellular enlargement and increased beta galactosidase activity were observed, representing a senescent phenotype (FIG. 4B), which is in agreement with other reports (Choi et al. (2012), supra; Vora et al. (2014), supra; Goel et al. (2016), supra; Puyol et al. (2010) supra; Witkiewicz et al. (2014), supra). Interestingly, cellular senescence has been associated with apoptosis resistance (Campisi & di Fagagna (2007) Nature Reviews Molecular Cell Biology 8:729-740). In this study, abemaciclib consistently reduced Poly ADP ribose polymerase cleavage in tumor cells and suppressed their apoptotic response to staurosporine (FIGS. 4C-4D). Therefore, CDK4/6 inhibitors do not induce apoptosis in tumor cells directly, but do enhance their susceptibility to T cell-mediated cytotoxicity.

Analysis of gene expression data from The Cancer Genome Atlas (TCGA) (e.g., see Cerami et al. (2012) Cancer Discov 2:401-404; Gao et al. (2013) Science Signaling 6:11) revealed that breast cancers harboring amplification of cyclin D1 (a mechanism for enhancing CDK4/6 activity) display significantly lower expression of TAP2, HLA-A, HLA-B, and HLA-C than non-amplified controls (FIG. 1I). These associations suggest that activity of the cyclinD:CDK4/6-Rb-E2F axis suppresses tumor cell antigen processing and presentation.

### Example 4: CDK4/6 Inhibition Induces Interferon Signaling

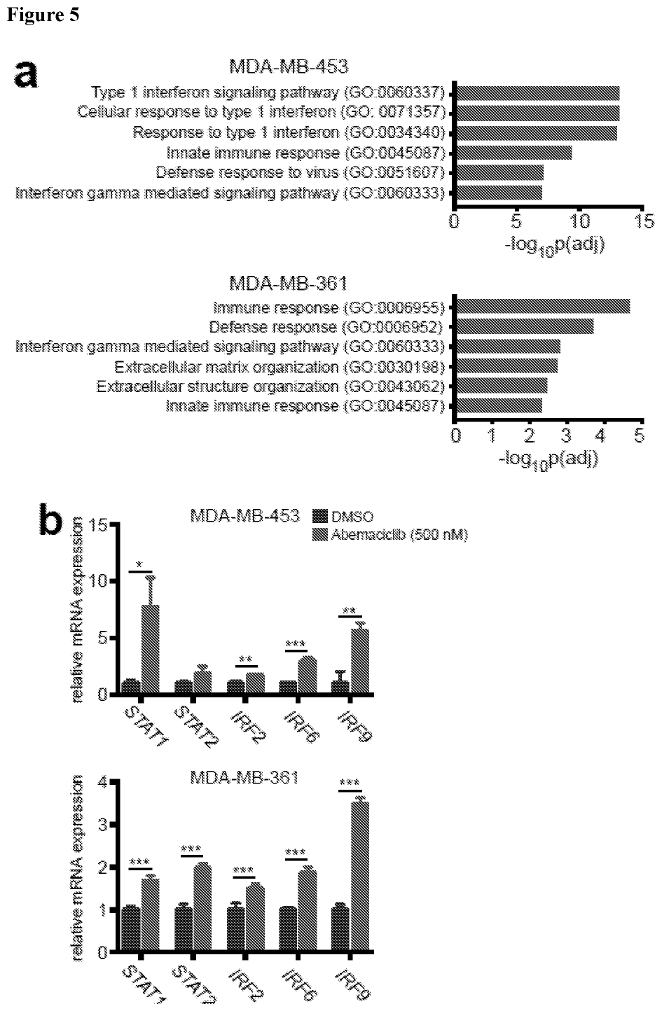

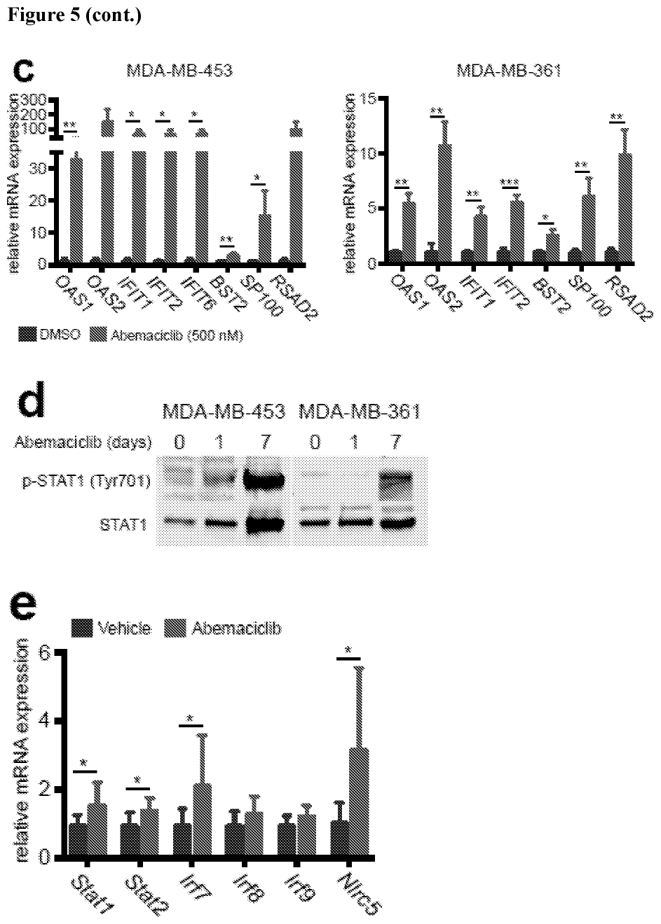

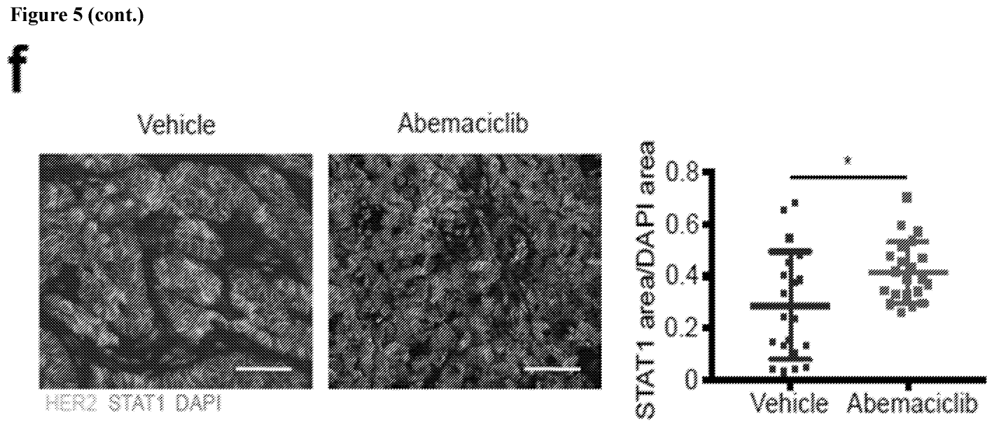

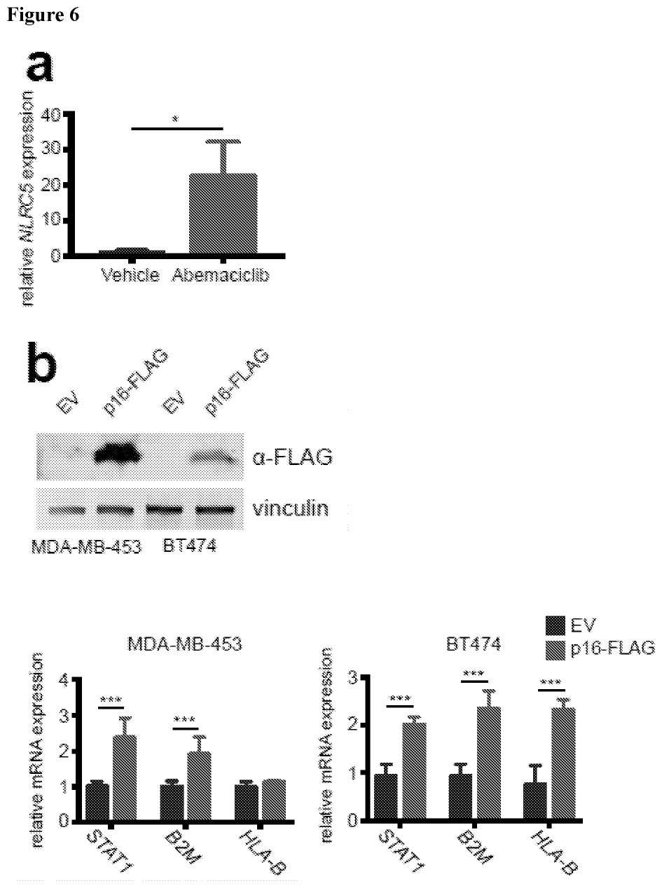

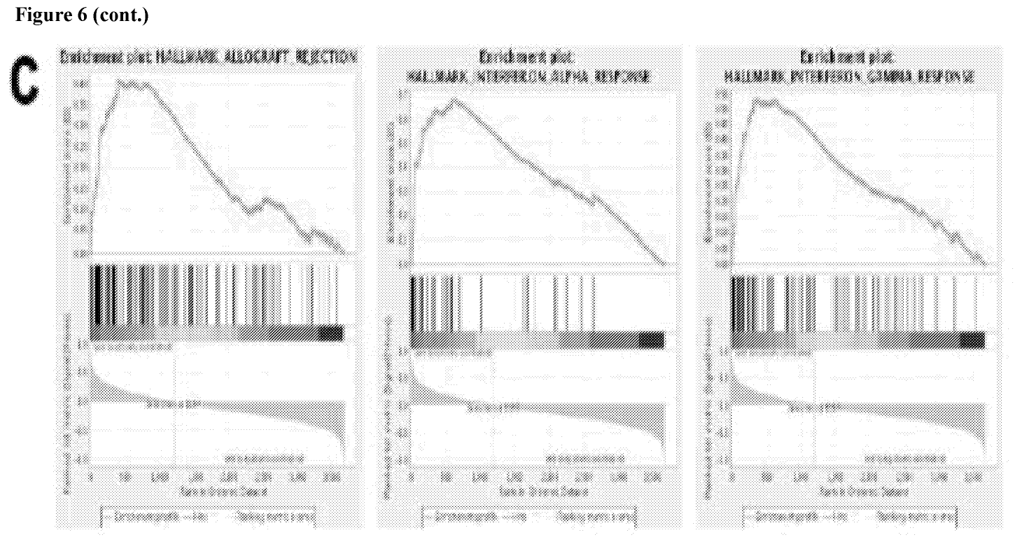

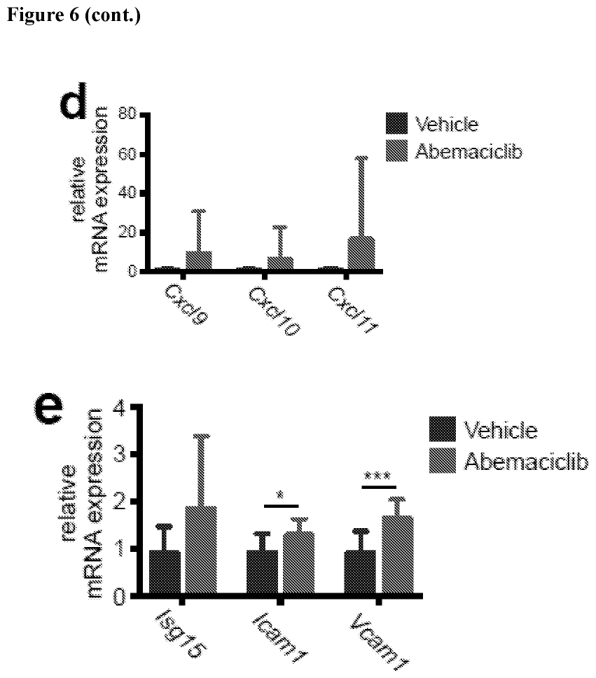

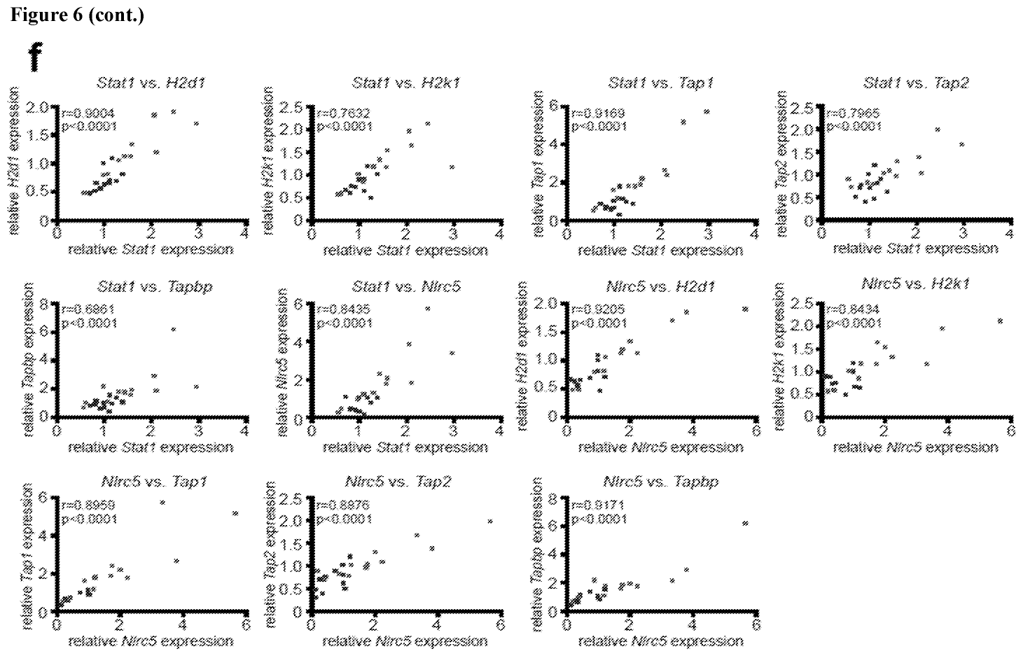

Genome-wide transcriptomic analysis was performed on breast cancer cell lines treated with abemaciclib or DMSO for 7 days. The top-ranked GO process terms enriched for upregulated genes in abemaciclib-treated MDA-MB-453 cells all pertained to interferon signaling and a cellular defense response to virus, including several MHC class I genes (FIG. 5A). Similarly, the highest ranked terms for MDA-MB-361 cells related to immune response, defense response, and interferon-mediated signaling (FIG. 5A). Several interferon-sensitive transcription factors (STAT1, STAT2, IRF2, IRF6, and IRF9) were upregulated greater than two-fold in both cell lines (FIG. 5B). Many of these factors activate expression of MHC class I genes and genes coding for the peptide processing machinery (van den Elsen (2011) Front Immunol 2:48). Additionally, a master regulator of MHC class I transcription, NLRC5, was upregulated in the MDA-MB-453 cells (FIG. 6A) (also see Meissner et al. (2010) Proc Natl Acad Sci USA 107:13794-13799). In agreement with a global upregulation of an interferon-driven transcriptional program, the expression of several other interferon-sensitive genes (ISGs)—OAS1, OAS2, IFIT1, IFIT2, IFIT6, BST2, SP100, RSAD2—was markedly enhanced (FIG. 5C). At the protein level, both phosphorylated and total STAT1 (a key mediator of interferon signaling) increased upon abemaciclib treatment in both cell lines (FIG. 5D). Importantly, these changes are likely “on-target” effects of CDK4/6 inhibitors, as forced overexpression of the endogenous CDK4/6 inhibitor CDKN2A (encoding p16^{INK4a}) also resulted in increased expression of STAT1, B2M, and MHC class I genes (FIG. 6B).

Consistent with the in vitro findings, genes upregulated in MMTV-rtTA/tetO-HER2 tumor tissue after abemaciclib treatment were enriched in the GSEA terms “allograft rejection”, “interferon alpha response”, and “interferon gamma response” (FIG. 6C). Specifically, abemaciclib treatment significantly increased expression of interferon-responsive transcription factors Stat1, Stat2, Irf7, and Nlrc5 (FIG. 5E), as well as the interferon-inducible T cell chemoattractants Cxcl9, Cxcl10, and Cxcl11 (FIG. 6D). Other transcription factors (Irf8, Irf9) were increased non-significantly (FIG. 5E). Furthermore, immunofluorescent staining of tumors revealed a significant increase in STAT1 protein within tumor cells of the abemaciclib-treated cohort (FIG. 5F). A number of other ISGs were overexpressed in vivo after abemaciclib treatment, including those involved in lymphocyte adhesion and co-stimulation (Icam1 and Vcam1) (FIG. 6E). The in vivo expression level of either STAT1 or NLRC5, both as regulators of antigen presentation via MHC class I, was tightly correlated with the levels of H2d1, H2d1, Tap1, Tap2, and Tapbp (FIG. 6F). Thus, CDK4/6 inhibition directly induces expression of a catalogue of ISGs in tumor cells, which explains their enhanced capacity for antigen presentation.

### Example 5: Interferon Signaling Associated with DNMT1 Suppression

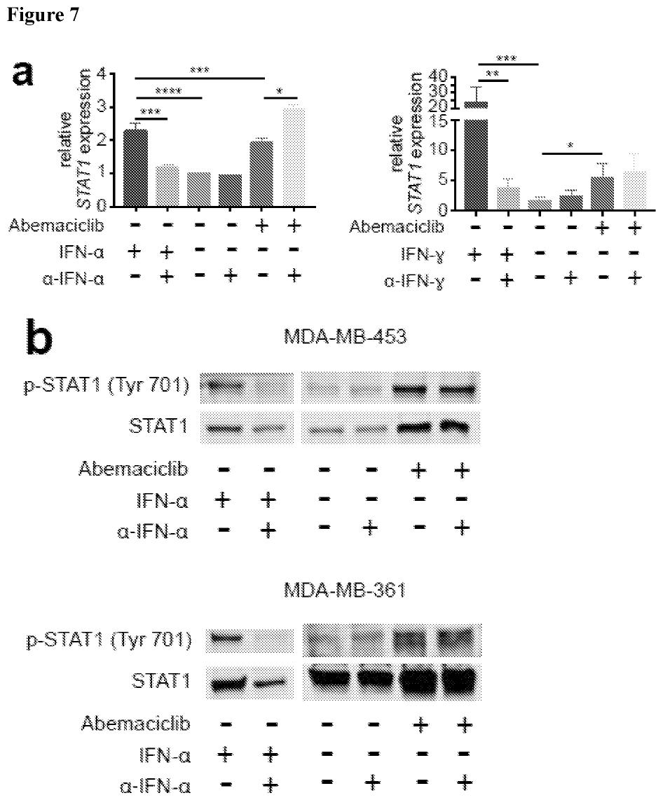

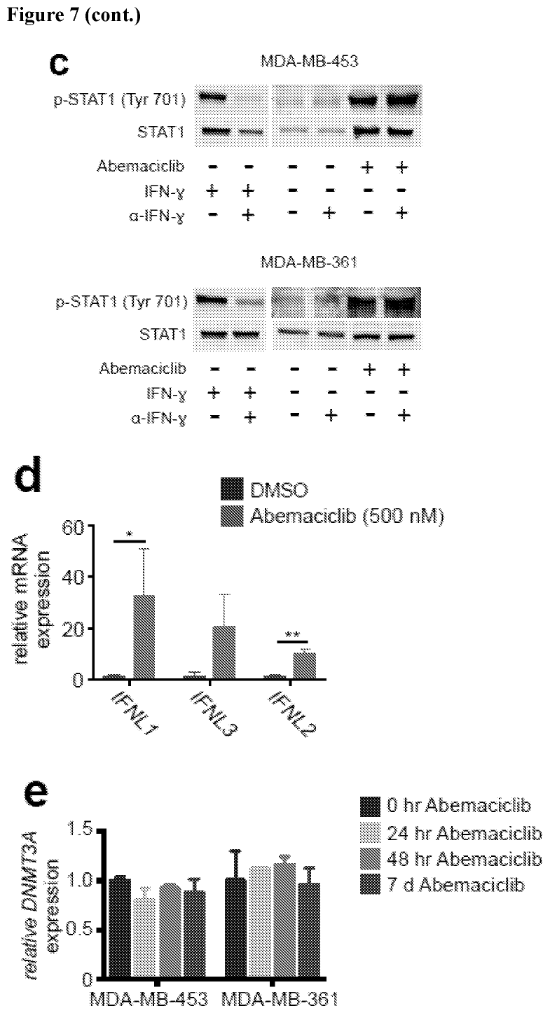

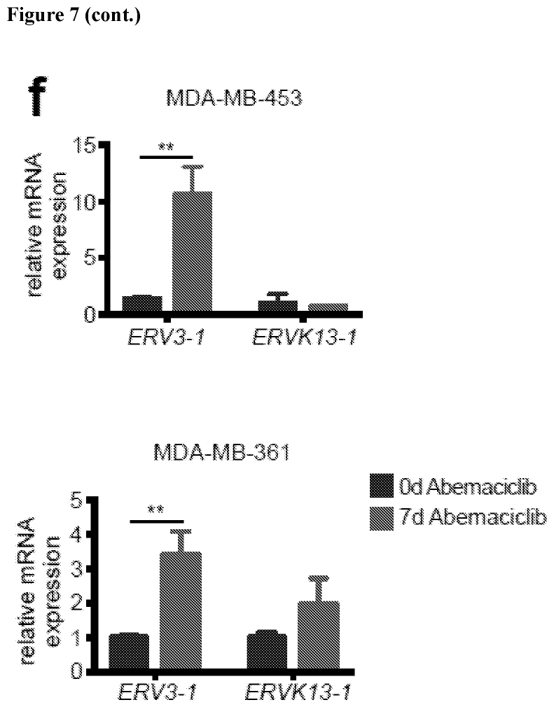

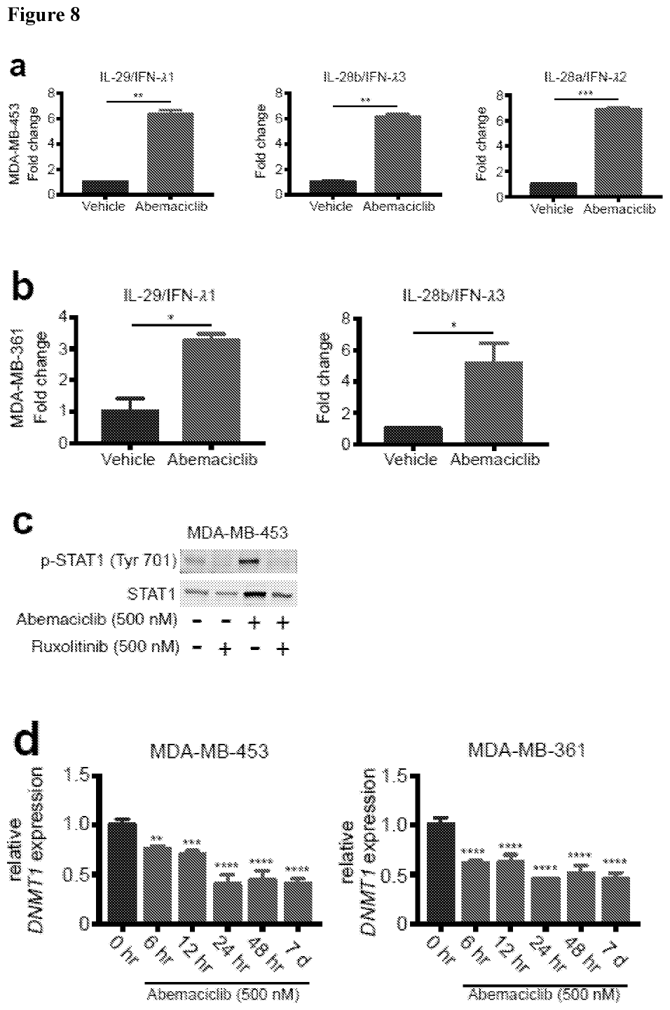

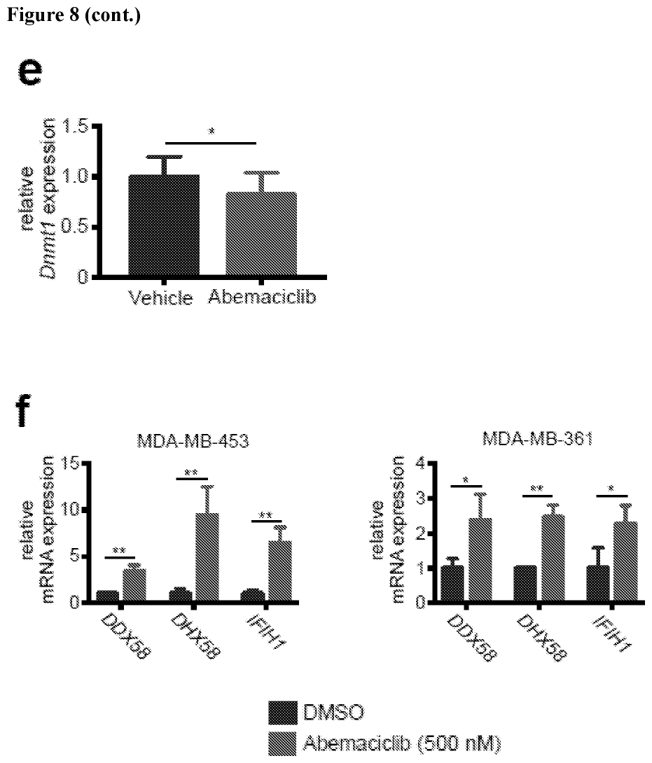

Interestingly, none of interferon alpha, beta, and gamma was detected in the conditioned medium of abemaciclib-treated tumor cells. Similarly, neutralizing antibodies against interferon alpha and interferon gamma did not mitigate interferon signaling in abemaciclib-treated cells, as measured by STAT1 mRNA and phosphorylated and total STAT1 protein (FIGS. 7A-7C). Instead, there was a significant increase of type III interferon levels in tumor cell conditioned medium after abemaciclib treatment. In MDA-MB-453 cells, abemaciclib increased production of IL-29, IL-28a, and IL-28b (i.e., IFN-λ1, IFN-λ2, and IFN-λ3, respectively) at both the mRNA and protein levels (FIGS. 7D and 8A). Similarly, abemaciclib increased IL-29 and IL-28b levels in MDA-MB-361 conditioned medium (IL-28a was not detected) (FIG. 8B). The Janus Kinases (JAKs) mediate intracellular signaling after ligand-dependent activation of interferon receptors (Parker et al. (2010) Nat Rev Cancer 16:131-144). In this study, a JAK inhibitor, ruxolitinib, completely mitigated abemaciclib-induced increases in p-STAT1 and total STAT1 levels in tumor cells (FIG. 8C). Collectively, these data suggested that CDK4/6 inhibition increases tumor cell production of type III interferons to drive ISG expression in an autocrine fashion.

Increased tumor cell interferon signaling via type III interferon production has recently been shown to occur as a consequence of DNA demethylating agents, such as 5-azacytidine, which inhibits DNA methyltransferases (DNMTs) (Roulois et al. (2015) Cell 162:961-973). In that context, inhibition of DNMTs reduces methylation of endogenous retroviral genes (ERVs), triggering “viral mimicry” and thence a double-stranded RNA (dsRNA) response. This in turn triggers type III interferon production, activating the expression of numerous ISGs (Roulois et al. (2015), supra). Notably, the primary mammalian DNMT (DNMT1) is also a bona fide E2F target gene, and CDK4/6 enzyme activity can enhance DNMT1 gene expression in an Rb-E2F dependent fashion (Kimura et al. (2003) Nucleic Acids Res 31:3101-3113). Strikingly, tumor cell DNMT1 expression fell markedly and rapidly with abemaciclib treatment (FIG. 8D). Other DNMTs had either very low expression levels (e.g., TRDMT1 and DNMT3B) or no change in expression levels after treatment with abemaciclib (DNMT3A) (FIG. 7E). Dnmt1 expression in MMTV-rtTA/tetO-HER2 tumor tissue was also reduced by abemaciclib therapy (FIG. 8E). Consistent with previous studies in Roulois et al. (2015), supra and Chiappinelli et al. (2015) Cell 162:974-986, the reduction in DNMT1 was associated with increased expression of ERV3-1 (in both cell lines) and ERVK13-1 (in MDA-MB-361 cells) (FIG. 7F). Furthermore, the expression of pattern recognition receptors for dsRNA RIG-1 (encoded by DDX58), LGP2 (encoded by DHX58), and MDA5 (encoded by IFIH1) was significantly increased in both cell lines after abemaciclib treatment for 7 days, indicating an associated dsRNA response (FIG. 8F).

Collectively, these data indicate that CDK4/6 inhibitor therapy reduces tumor cell DNMT1 expression, which is associated with enhanced ERV expression, a dsRNA response, and type III interferon secretion. As a result, multiple ISG express to enhance antigen presentation.

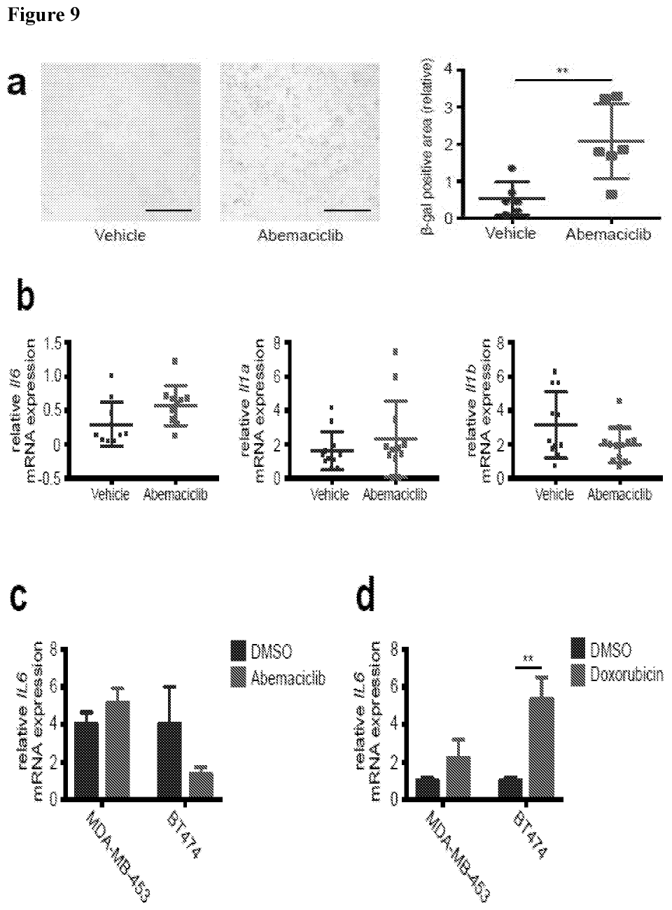

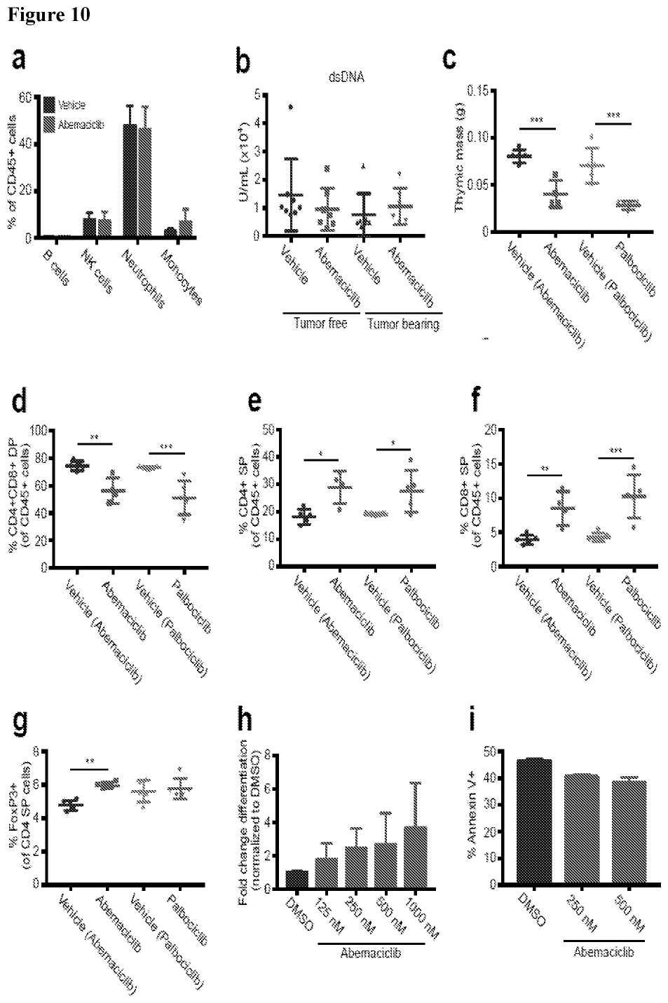

In some contexts, a senescence associated secretory phenotype (SASP) can also facilitate immune responses (Coppe et al. (2010) Annu Rev Pathol 5:99-118; Xue et al. (2007) Nature 445:656-60; Iannello et al. (2013) J. Exp. Med. 210:2057-2069). Hence, given that abemaciclib increased beta galactosidase in vitro and in vivo (FIGS. 4B and 9A), evidence of SASP was sought. Expression of principal SASP factors Il-6, Il-1a, and Il-1b did not significantly increase after abemaciclib treatment (FIG. 9B-9C and Table 2). Furthermore, expression analysis of a larger panel of SASP genes did not show a change for most leukocyte chemotactic factors (Table 2). In contrast, doxorubicin treatment of tumor cells increased beta galactosidase and interleukin-6 expression (FIG. 9D), consistent with the notion that the SASP is specific to DNA damage response-associated senescence (Coppe et al. (2010), supra).

TABLE 2
Relative expression of SASP genes in cancer cells and turmors
A. Gene expression in MDA-MB-453 cells after
abemaciclib or DMSO treatment

| SASP gene | Fold change | |
|---|---|---|
| (>20 reads in | (abema vs | p value |
| any sample) | DMSO treatment) | (unadjusted) |
| COL18A1 | 2.14308977 | 0.628025454 |
| COL1A1 | 0.751952379 | 0.284665275 |
| CTSB | 1.319906094 | 0.182089524 |
| EGF | 10.38537043 | 0.034033282 |
| EGFR | 1.755402285 | 0.210308062 |
| ICAM1 | 2.077293749 | 0.073024515 |
| IGFBP3 | 1.462780156 | 0.046471908 |
| IGFBP4 | 0.496123198 | 0.057239164 |
| IGFBP5 | 1.566771836 | 0.395853593 |
| KITLG | 1.643409803 | 0.182654202 |
| LAMA5 | 1.681302802 | 0.024009926 |
| LAMB1 | 0.494157967 | 0.010990484 |
| LAMB2 | 3.347286734 | 0.018753777 |
| LAMB3 | 1.547544086 | 0.426166051 |
| LAMC1 | 2.374592887 | 0.179689152 |
| MMP13 | 6.371474851 | 0.039579357 |
| TIMP1 | 1.75601697 | 0.169899825 |
| TNFRSF1A | 0.955267238 | 0.659083377 |
| VEGFA | 2.228713975 | 0.006925791 |

* All tested SASP genes include: ANG, AREG, CCL1, CCL11, CCL13,
CCL16, CCL2, CCL20, CCL25, CCL26, CCL3, CCL8, COL10A1,
COL11A1, COL18A1, COL1A1, COL1A2, COL2A1, COL3A1,
COL4A1, COL4A2, COL4A3, COL5A1, COL5A2, COL6A1, COL8A2,
COL9A1, CSF2, CSF3, CTSB, CXCL1, CXCL11, CXCL12, CXCL13,
CXCL2, CXCL3, CXCL5, EGF, EGFR, EREG, FAS, FGF2, FGF7,
FN1, HGF, ICAM1, ICAM3, IFNG, IGF2BP2, IGFBP3, IGFBP4,
IGFBP5, IGFBP6, IGFBP7, IL13, IL15, ILIA, IL1B, IL6,
IL6ST, IL7, IL8, KITLG, LAMA1, LAMA2, LAMA3, LAMA5,
LAMB1, LAMB2, LAMB3, LAMC1, MMP10, MMP12, MMP13,
MMP14, MMP3, NGF, NOS2, NOS3, NRG1, PGF, PLAT, PLAU,
PLAUR, SERPINB2, SERPINE1, TIMP1, TIMP2, TNFRSF10C,
TNFRSF11B, TNFRSF1A, TNFRSF1B, and VEGFA.

B. Gene expression in MDA-MB-361 cells after
abemaciclib or DMSO treatment

| SASP gene | Fold change | |
|---|---|---|
| (>20 reads in | (abema vs | p value |
| any sample) | DMSO treatment) | (unadjusted) |
| AREG | 0.626681127 | 0.055284433 |
| COL18A1 | 1.571857267 | 0.137385603 |
| COL1A1 | 1.170036717 | 0.339197062 |
| COL5A1 | 1.560837456 | 0.324326246 |
| COL6A1 | 1.332350379 | 0.335634375 |
| COL9A1 | 1.225731662 | 0.978083889 |
| CTSB | 1.780801304 | 0.016081846 |
| CXCL12 | 1.443999821 | 0.0448026 |
| EGF | 1.385812746 | 0.037825211 |
| EGFR | 0.75992877 | 0.013259226 |
| FN1 | 0.195639358 | 5.05426E−05 |
| IGFBP3 | 7.090709478 | 0.000101921 |
| IGFBP4 | 1.45606665 | 0.040802808 |
| IGFBP5 | 3.815824804 | 0.000249738 |
| IL6ST | 1.449500336 | 0.13771422 |
| IL8 | 0.159803453 | 0.007373674 |
| KITLG | 2.541006002 | 0.004782748 |
| LAMA3 | 1.243734528 | 0.455981486 |
| LAMA5 | 1.348551162 | 0.038245301 |
| LAMB1 | 1.197727245 | 0.494121669 |
| LAMB2 | 2.455238241 | 0.017673056 |
| LAMB3 | 1.996357064 | 6.94597E−05 |
| LAMC1 | 1.778403907 | 0.000667982 |
| TIMP1 | 2.287394858 | 0.064456848 |
| TIMP2 | 0.625109788 | 0.083216225 |
| TNFRSF1A | 1.098767024 | 0.713927362 |
| VEGFA | 1.552414463 | 0.008613273 |

* All tested SASP genes include: ANG, AREG, CCL1, CCL11, CCL13,
CCL16, CCL2, CCL20, CCL25, CCL26, CCL3, CCL8, COL10A1,
COL11A1, COL18A1, COL1A1, COL1A2, COL2A1, COL3A1,
COL4A1, COL4A2, COL4A3, COL5A1, COL5A2, COL6A1,
COL8A2, COL9A1, CSF2, CSF3, CTSB, CXCL1, CXCL11,
CXCL12, CXCL13, CXCL2, CXCL3, CXCL5, EGF, EGFR,
EREG, FAS, FGF2, FGF7, FN1, HGF, ICAM1, ICAM3, IFNG,
IGF2BP2, IGFBP3, IGFBP4, IGFBP5, IGFBP6, IGFBP7,
IL13, IL15, ILIA, IL1B, IL6, IL6ST, IL7, IL8,
KITLG, LAMA1, LAMA2, LAMA3, LAMA5, LAMB1, LAMB2,
LAMB3, LAMC1, MMP10, MMP12, MMP13, MMP14, MMP3,
NGF, NOS2, NOS3, NRG1, PGF, PLAT, PLAU, PLAUR,
SERPINB2, SERPINE1, TIMP1, TIMP2, TNFRSF10C,
TNFRSF11B, TNFRSF1A, TNFRSF1B, and VEGFA.

C. Gene expression MMTV-HER2 tumors after
abemaciclib or DMSO treatment

| SASP gene | Fold change | |
|---|---|---|
| (>20 reads in | (abema vs | p value |
| any sample) | DMSO treatment) | (unadjusted) |
| ANG | 1.225676662 | 0.474729975 |
| CCL11 | 1.355312179 | 0.176746265 |
| CCL2 | 1.984494667 | 0.252633898 |
| CCL20 | 0.884611074 | 0.655722436 |
| CCL25 | 1.158704207 | 0.326603365 |
| CCL3 | 1.482799881 | 0.056400123 |
| CCL8 | 1.138102405 | 0.649419739 |
| COL10A1 | 0.798950747 | 0.443540357 |
| COL11A1 | 0.681068162 | 0.003866833 |
| COL18A1 | 0.885686324 | 0.531802755 |
| COL1A1 | 1.206588482 | 0.522569179 |
| COL1A2 | 1.245276214 | 0.543604777 |
| COL2A1 | 1.183695134 | 0.82897355 |
| COL3A1 | 0.875849254 | 0.596687343 |
| COL4A1 | 0.792593409 | 0.089654835 |
| COL4A2 | 0.908381442 | 0.428483522 |
| COL5A1 | 0.845559755 | 0.420192783 |
| COL5A2 | 1.155414641 | 0.409434479 |
| COL6A1 | 0.974265193 | 0.873783785 |
| CSF3 | 0.927908765 | 0.761531034 |
| CTSB | 1.237377178 | 0.014910104 |
| CXCL1 | 0.909178034 | 0.70556841 |
| CXCL11 | 16.38413114 | 0.234032762 |
| CXCL12 | 0.808561619 | 0.263019649 |
| CXCL2 | 1.863609232 | 0.006107586 |
| CXCL3 | 2.480880833 | 0.114361505 |
| CXCL5 | 1.274664521 | 0.689493076 |
| EGFR | 1.039344676 | 0.694770476 |
| FGF2 | 1.08743053 | 0.65752356 |
| FGF7 | 0.659732088 | 0.310191313 |
| FN1 | 1.00762147 | 0.973856619 |
| HGF | 1.349975221 | 0.031421068 |
| ICAM1 | 1.287020321 | 0.04224503 |
| IGF2BP2 | 2.002130473 | 0.149443123 |
| IGFBP3 | 0.727825291 | 0.113465842 |
| IGFBP4 | 0.935344589 | 0.725841333 |
| IGFBP5 | 0.842654986 | 0.12693988 |
| IGFBP6 | 1.084334646 | 0.737734135 |
| IGFBP7 | 0.922164284 | 0.61510165 |
| IL15 | 0.868151561 | 0.379574856 |
| IL1A | 1.433442413 | 0.362976826 |
| IL1B | 0.619676155 | 0.076968622 |
| IL6ST | 1.072873755 | 0.341378727 |
| IL7 | 0.741403367 | 0.230754151 |
| LAMA1 | 0.907453157 | 0.576504871 |
| LAMA2 | 1.808202824 | 0.023997141 |
| LAMA3 | 1.061209015 | 0.826995245 |
| LAMA5 | 0.81881378 | 0.29454451 |
| LAMB1 | 1.027980171 | 0.887586172 |
| LAMB2 | 0.900448643 | 0.146759198 |
| LAMB3 | 0.918256879 | 0.49400467 |
| LAMC1 | 0.887640499 | 0.333522302 |
| MIF | 0.821602736 | 0.177271457 |
| MMP10 | 1.444959346 | 0.218881103 |
| MMP12 | 1.308150612 | 0.274337613 |
| MMP13 | 2.388118846 | 0.026331234 |
| MMP14 | 1.088668452 | 0.663022658 |
| MMP3 | 2.887064187 | 0.001332675 |
| NGF | 1.188905392 | 0.772140802 |
| NOS2 | 1.661114766 | 0.491573439 |
| NOS3 | 0.972162007 | 0.886022832 |
| NRG1 | 1.497982399 | 0.210363913 |
| PGF | 0.678750771 | 0.060194457 |
| PLAT | 1.242634169 | 0.334934406 |
| PLAU | 1.237701789 | 0.359306964 |
| PLAUR | 1.507013674 | 0.178174041 |
| SERPINB2 | 11.99781166 | 0.102304859 |
| SERPINE1 | 1.231163282 | 0.347113673 |
| TIMP1 | 1.270977274 | 0.420901109 |
| TIMP2 | 1.094449422 | 0.348606893 |
| TNFRSF11B | 0.88031306 | 0.649191827 |
| TNFRSF1A | 0.958456528 | 0.547887704 |
| TNFRSF1B | 1.195587742 | 0.097096191 |
| VEGFA | 0.874723947 | 0.596983024 |

* All tested SASP genes include: ANG, AREG, CCL1, CCL11, CCL2,
CCL20, CCL25, CCL26, CCL3, CCL8, COL10A1, COL11A1,
COL18A1, COL1A1, COL1A2, COL2A1, COL3A1, COL4A1, COL4A2,
COL4A3, COL5A1, COL5A2, COL6A1, COL8A2, COL9A1, CSF2,
CSF3, CTSB, CXCL1, CXCL11, CXCL12, CXCL13, CXCL2, CXCL3,
CXCL5, EGF, EGFR, FAS, FGF2, FGF7, FN1, GENE, HGF, ICAM1,
IFNG, IGF2BP2, IGFBP3, IGFBP4, IGFBP5, IGFBP6, IGFBP7,
IL13, IL15, IL1A, IL1B, IL6, IL6ST, IL7, LAMA1, LAMA2,
LAMA3, LAMA5, LAMB1, LAMB2, LAMB3, LAMC1, MIF,
MMP10, MMP12, MMP13, MMP14, MMP1A, MMP1B, MMP3, NGF,
NOS2, NOS3, NRG1, PGF, PLAT, PLAU, PLAUR,
SERPINB2, SERPINE1, TIMP1, TIMP2, TNFRSF11B,
TNFRSF1A, TNFRSF1B, and VEGFA.
* Table 2 summarizes the expression of SASP genes in MDA-MB-453 (Table 2A) and MDA-MB-361 (Table 2B) cells treated with abemaciclib (500 nM) for 7 days relatve to DMSO, or the expression of SASP genes in MMTV-rtTA/tetO-HER2 tumors (Table 2C) treated with abemaciclib for 12 days relative to vehicle, as determined by transcriptomic analysis.

### Example 6: CDK4/6 Inhibition Suppresses Treg Proliferation

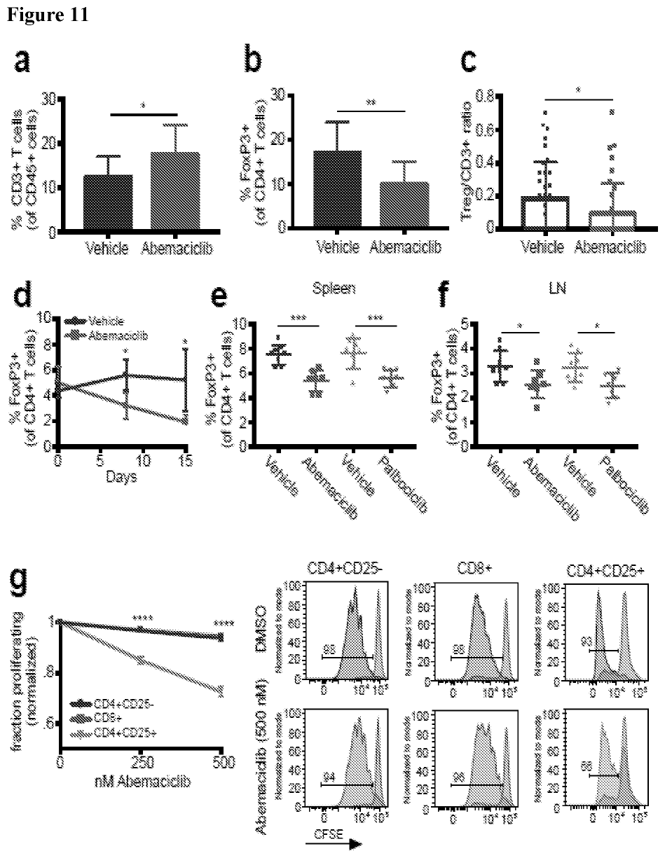

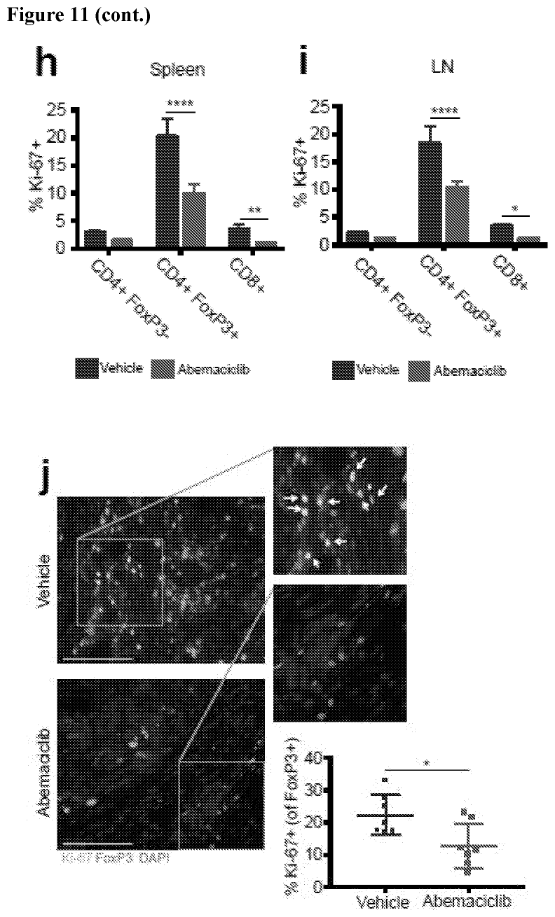

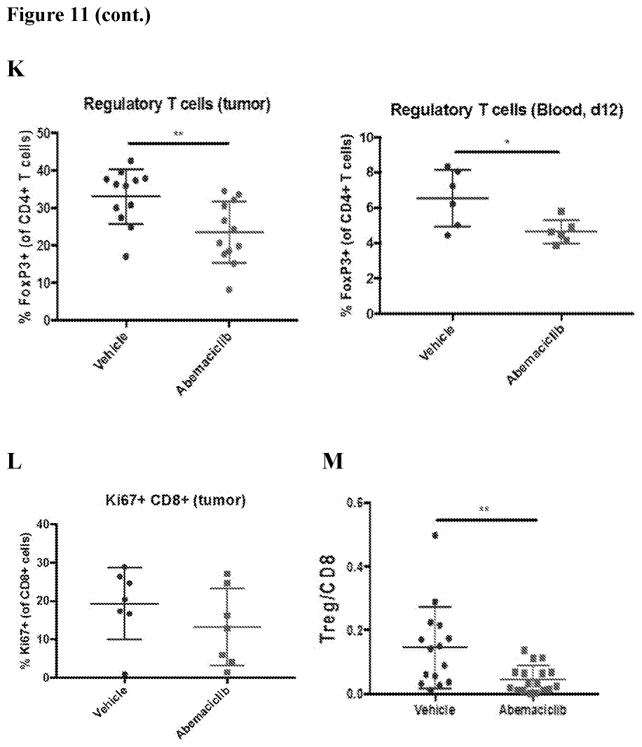

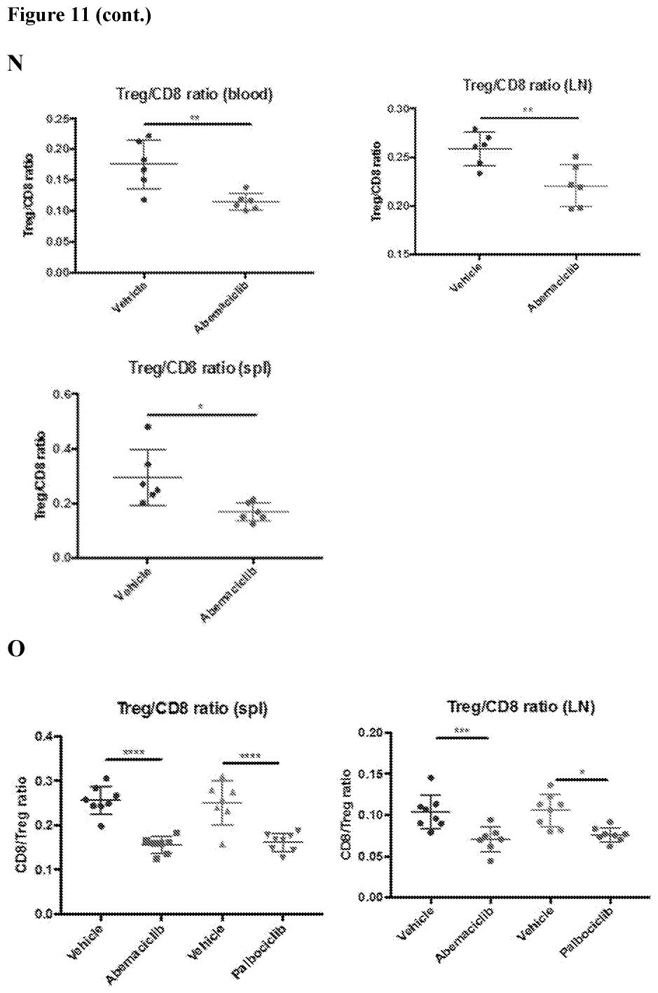

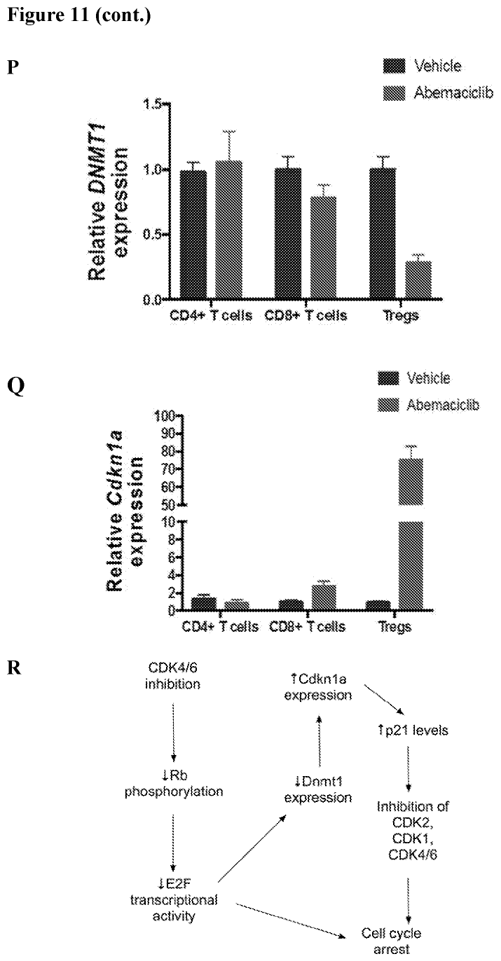

The discovery that CDK4/6 inhibition enhances tumor cell antigen presentation prompted examination of the immune microenvironment. Tumor-bearing WTV-rtTA/tetO-HER2 mice were treated with abemaciclib or vehicle for 12 days and then performed flow cytometry on tumor tissue. Extensive immune profiling revealed that abemaciclib did not alter the fraction of tumor-infiltrating B lymphocytes, natural killer cells, neutrophils, or monocytes (FIG. 10A). Strikingly, however, there was a marked and significant increase in numbers of infiltrating CD3+ T cells (FIG. 11A), which was not specific to the CD4+ or CD8+ population. In addition, there was a significant reduction in the proportion of CD4+FOXP3+ regulatory T cells (Tregs) (FIG. 11B), which are an immunosuppressive cell population that constrains anti-tumor immunity (Plitas & Rudensky (2016) Cancer Immunol Res 4:721-725). Moreover, the intra-tumoral ratio of Tregs to CD3+ cells decreased with abemaciclib treatment (FIG. 11C).

A significant reduction in circulating Tregs was observed in tumor-bearing mice treated with abemaciclib (FIG. 11D). In tumor-free mice, both abemaciclib and palbociclib significantly reduced Treg numbers in the spleen and lymph nodes, demonstrating tumor-independent effects of abemaciclib (FIGS. 11E-11F). No associated increases in an autoimmune phenotype were detected, as determined by anti-nuclear antibody (ANA) or anti-double stranded DNA antibody (dsDNA) levels (FIG. 10B).

For effects of CDK4/6 inhibition on natural Treg development in the thymus, palbociclib and abemaciclib each significantly reduced total thymic mass, decreased immature CD4+CD8+ double-positive thymocytes, and increased the fraction of CD4+ or CD8+ single-positive lymphocytes (FIGS. 10C-10F), consistent with previous reports of CDK6 inhibition (Malumbres et al. (2004) Cell 118:493-504). However, there was no reduction in the thymic Treg population, arguing against a defect in natural Treg production (FIG. 10G). Similarly, CDK4/6 inhibitors did not prevent differentiation of naïve CD4+ T cells into Tregs in vitro after stimulation with TGF-β (rather, there was a trend toward increased differentiation), and did not affect rates of Treg apoptosis (FIGS. 10H-10I).

For effects of CDK4/6 inhibition on proliferation of various T cell populations, Tregs (CD4+CD25+), CD8+ T cells, and CD4+CD25− T cells from spleens and lymph nodes of wild-type mice were isolated and treated with DMSO or abemaciclib in vitro. Strikingly, there was a selective suppression of Treg proliferation, which was not apparent in the CD8+ or CD4+CD25− populations (FIG. 11G). Similar observations were made with cells isolated from spleens and lymph nodes of tumor-free mice treated with abemaciclib in vivo, whereby Treg proliferation was significantly reduced (FIGS. 11H-11I). CD8+ T cell proliferation was also somewhat reduced in these tissues. However, their basal proliferation rate (approximately 4% Ki-67+) was much lower than that of the Treg population (approximately 20% Ki-67+). Thus, the absolute impact of their reduction on the overall population would be expected to be less profound. Consistent with these results, the fraction of proliferating Tregs within tumor tissue was significantly reduced by abemaciclib treatment (FIG. 11J). Thus, CDK4/6 inhibitors selectively suppress Treg proliferation, resulting in fewer immunosuppressive Tregs within tumors.

Similar Treg inhibition by CDK4/6 inhibitors was also found in other cancer models. For example, in mice bearing CT-26 colorectal carcinomas, CDK4/6 inhibition (e.g., by abemaciclib) reduced Treg numbers in the tumor and blood (FIG. 11K). As mentioned above, the CDK4/6 inhibition specifically reduced the proliferation of Treg but no other T cells, such as CD8+ T cells. As in FIG. 11L, double staining of MMTV-rtTA/tetO-HER2 tumors showed no significant reduction in the number of Ki67+CD8+ T cells after abemaciclib treatment for 12 days. Consistently, CDK4/6 inhibitors reduced Treg:CD8+ T cell ratio in mice having MMTV-rtTA/tetO-HER2 tumors (FIG. 11M) or CT-26 tumors (FIG. 11N). Such reduction in the Treg:CD8+ ratio is tumor-independent, since the similar reduction was found in the spleens and lymph nodes of tumor-free FVB mice after CDK4/6 inhibitor treatment for 12 days (FIG. 11O).

Furthermore, CDK4/6 inhibition (e.g., by abemaciclib) suppressed Dnmt1 expression in Tregs cells but no other T cells (e.g., CD4+ T cells or CD8+ T cells) (FIG. 11P). Such suppression was associated with an increased expression of CDKN1A in Treg cells in spleens and lymph nodes (FIG. 11Q). Without limitation, a proposed mechanism for suppression of Treg proliferation by CDK4/6 inhibitors is summarized in FIG. 11R.

### Example 7: Cytotoxic T Cells Mediate Tumor Regression

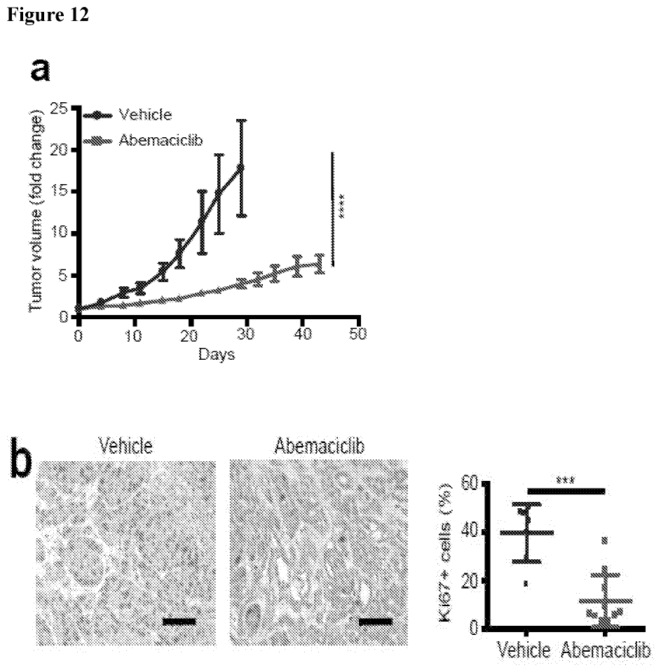

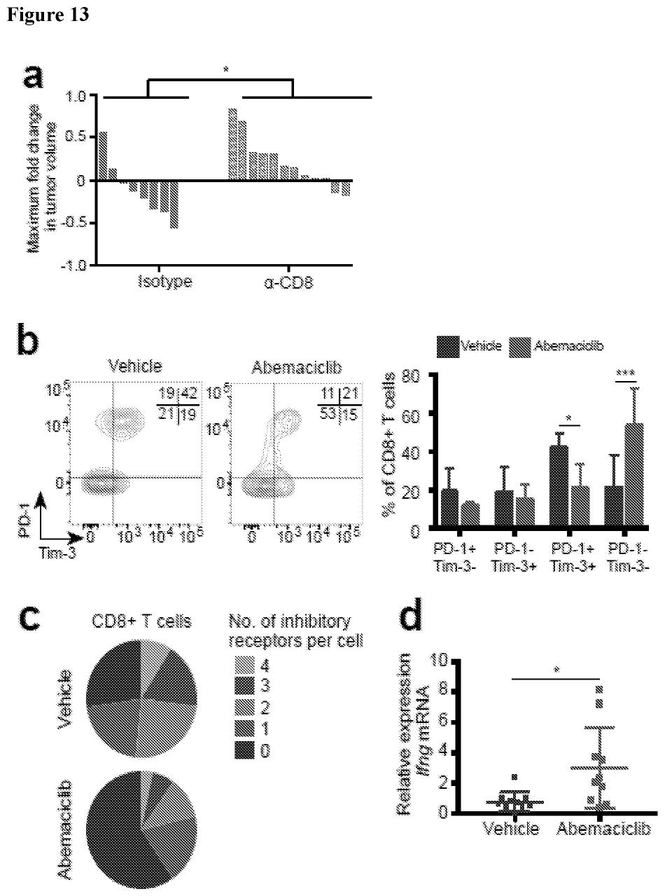

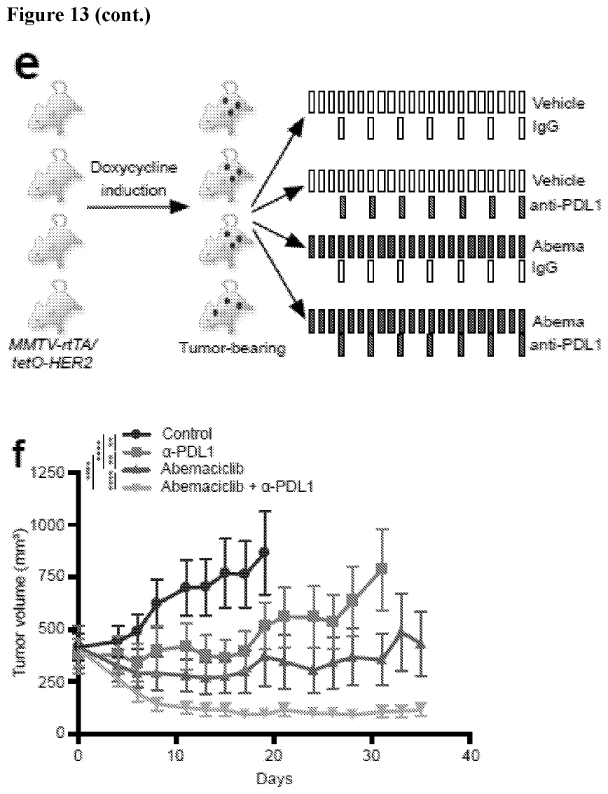

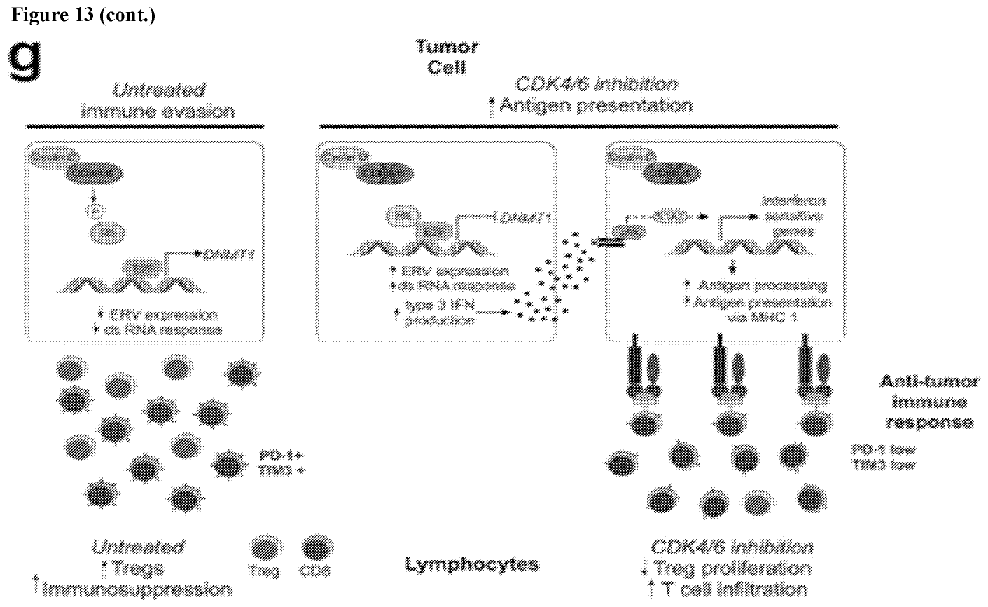

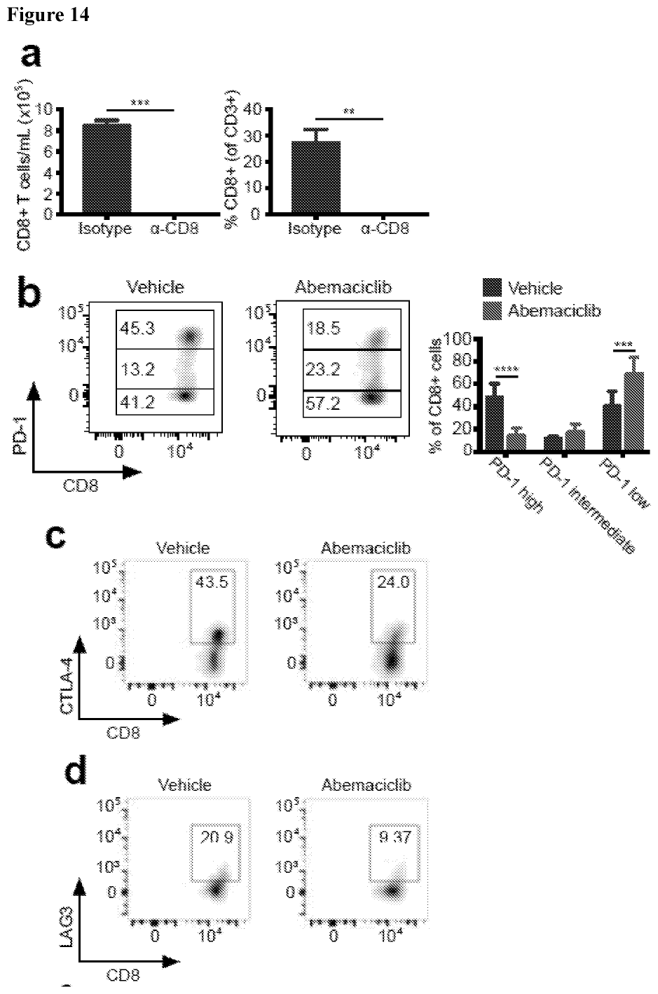

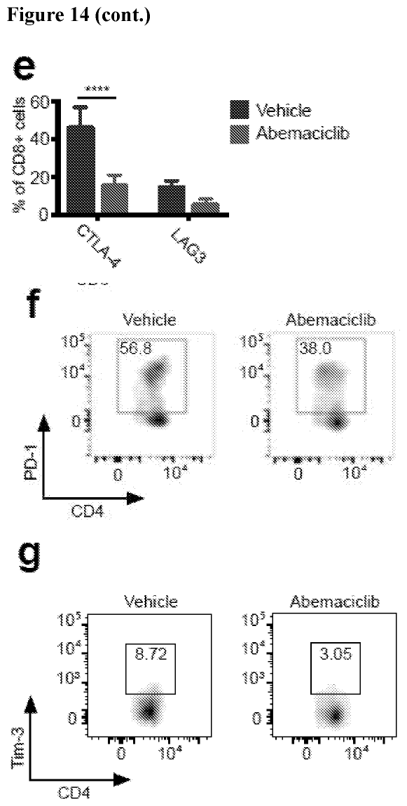

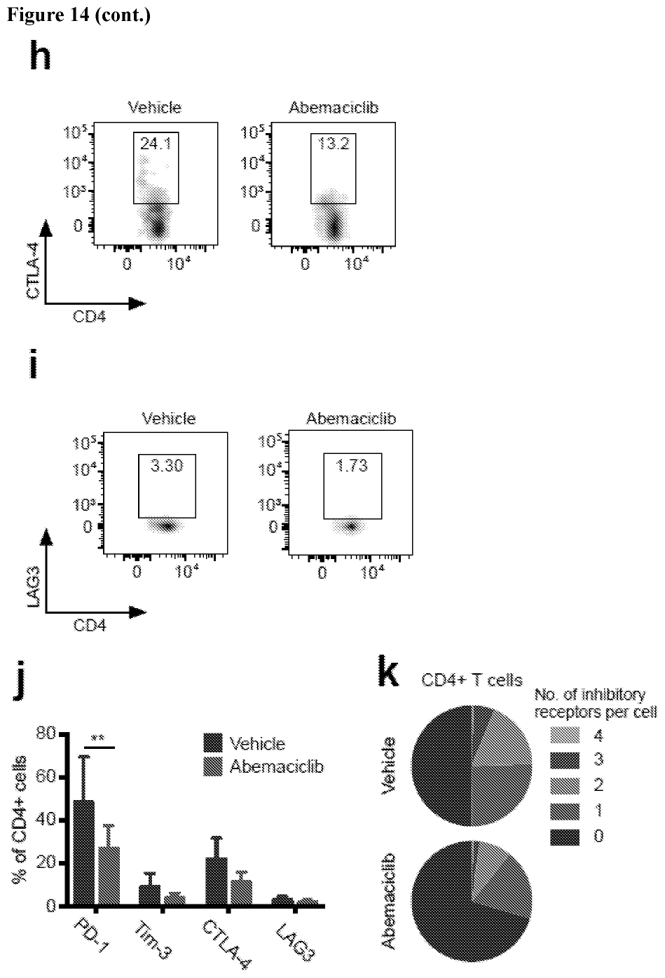

Given that tumor cells presenting antigen via MHC class I can be recognized by cytotoxic T lymphocytes (CTLs) and that Tregs suppress CTL efficacy by promoting their exhaustion (Penaloza-MacMaster et al. (2014) J Exp Med 211:1905-1918; Bauer et al. (2014) J Clin Invest 124:2425-2440), it was hypothesized that tumor regression after abemaciclib therapy might be mediated by CTLs. MMTV-rtTA/tetO-HER2 tumor fragments were implanted orthotopically into athymic Foxn1^{nu} mice maintained on a doxycycline diet. The established tumors were treated with abemaciclib or vehicle. In contrast to tumors in immune competent mice, abemaciclib-treated tumors in nude mice continued to grow, albeit at a significantly slower rate than vehicle-treated tumors (confirmed in three independent experiments, FIG. 12A). In no case did tumors regress with abemaciclib-treatment. In fact, abemaciclib-treated tumors were over 5-fold larger than they were at implantation after 45 days of treatment (FIGS. 12A-12B), even though cellular proliferation was suppressed to a similar extent as seen in immune competent mice (FIG. 12B). MMTV-rtTA/tetO-HER2 tumor-bearing mice were then treated with an anti-CD8 neutralizing antibody prior to administration of abemaciclib. As result, tumor regression was significantly mitigated (FIGS. 13A and 14A). Hence, tumor regression mediated by CDK4/6 inhibition was almost entirely dependent on the presence of cytotoxic T lymphocytes.

Further supporting the role of cytotoxic CD8+ T cells in mediating responses to CDK4/6 inhibition, tumor-infiltrating CD8+ cytotoxic T cells in abemaciclib-treated tumors showed markedly reduced cell-surface expression of the T cell exhaustion markers PD-1, Tim-3, CTLA-4, and LAG3 (FIGS. 13B-13C and 14B-14E). In fact, the number of inhibitory receptors detected on any given cytotoxic T cell was lower in abemaciclib treated tumors, with over 50% of the CD8+ cells expressing none of these (FIG. 13C). More specifically, the fractions of both PD-1^{high} and PD-1+/TIM-3+ cytotoxic T cells (profiles indicative of exhaustion, as shown in Jin et al. (2010) Proc Natl Acad Sci USA 107:14733-14738) were significantly reduced, the latter by 50 percent (FIGS. 13B and 14B). Furthermore, the mRNA level of Ifng (the main effector cytokine of cytotoxic T cells) was more than 4-fold higher in treated bulk tumor tissue (FIG. 13D).

CD4+ T cells also showed reductions in the cell-surface expression of PD-1, TIM-3, CTLA-4, and LAG-3. However, only the reduction in PD-1 was statistically significant (FIGS. 14F-14K). Collectively, these results established that CDK4/6 inhibition increases the number and decreases the degree of exhaustion of tumor-infiltrating CTLs, and that these cells are required for abemaciclib-induced tumor regression.

### Example 8: Combined CDK4/6 Inhibition and Checkpoint Blockade

Given that abemaciclib increased tumor cell antigen presentation and induced an anti-tumor T cell response, it was then tested whether adding immune checkpoint blockade to abemaciclib therapy could further enhance tumor regression. MMTV-rtTA/tetO-HER2 tumor bearing mice, which is a model of luminal breast cancer, were treated with either vehicle or abemaciclib, and a control IgG or an anti-PD-L1 antibody using a 2×2 randomization (FIG. 13E). As seen with initial experiments, the volume of abemaciclib treated tumors decreased (average 35 percent reduction at day 13 of treatment). With more prolonged therapy, these tumors stabilized and by day 21 several had begun to increase in volume again (FIG. 13F). In striking contrast, tumors in mice treated with combined abemaciclib plus anti-PD-L1 therapy regressed to a greater degree (average of 70 percent reduction in tumor volume by day 13), and showed no resumption of tumor growth by day 35 (FIG. 13F).

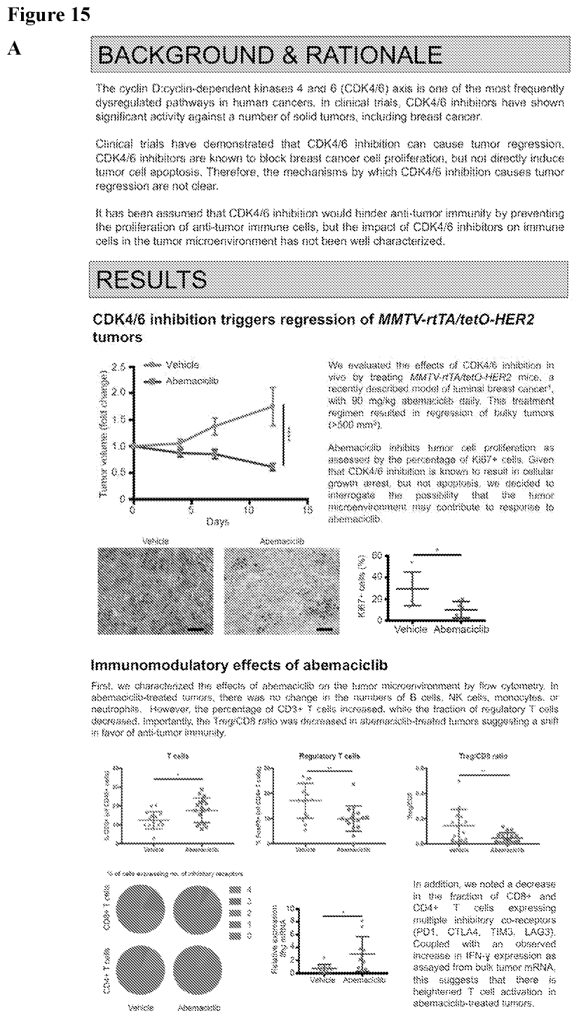

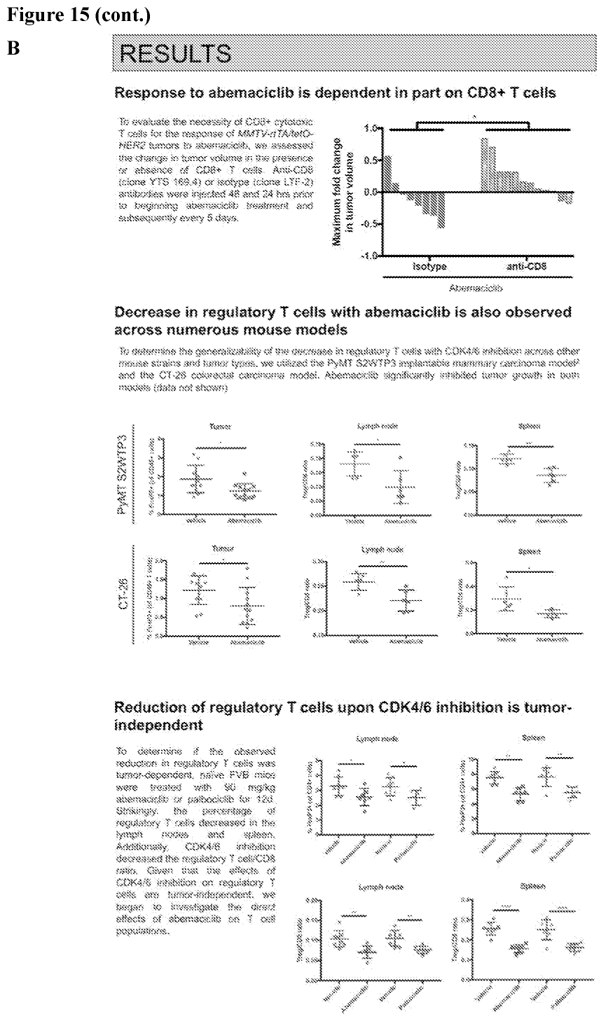

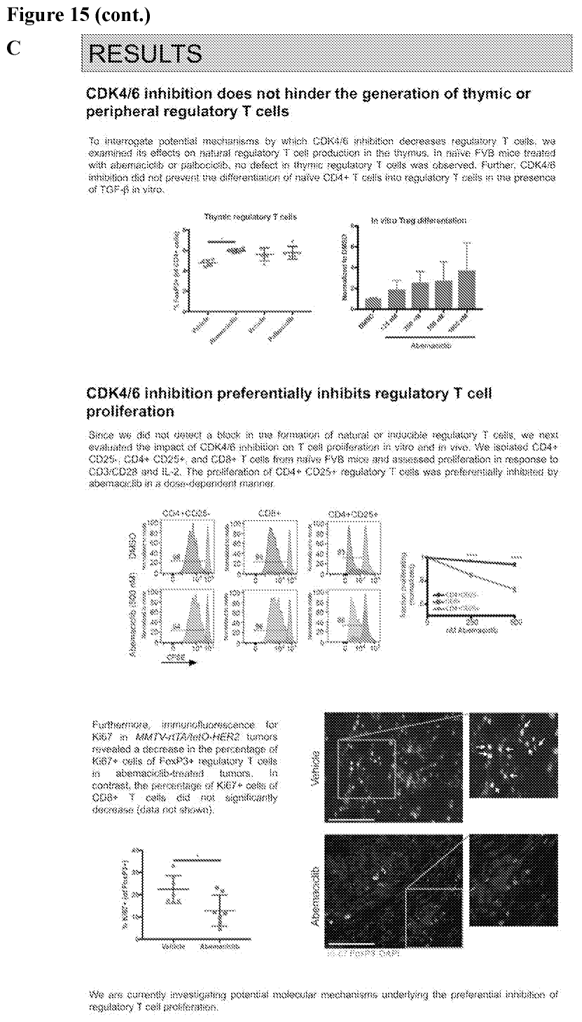

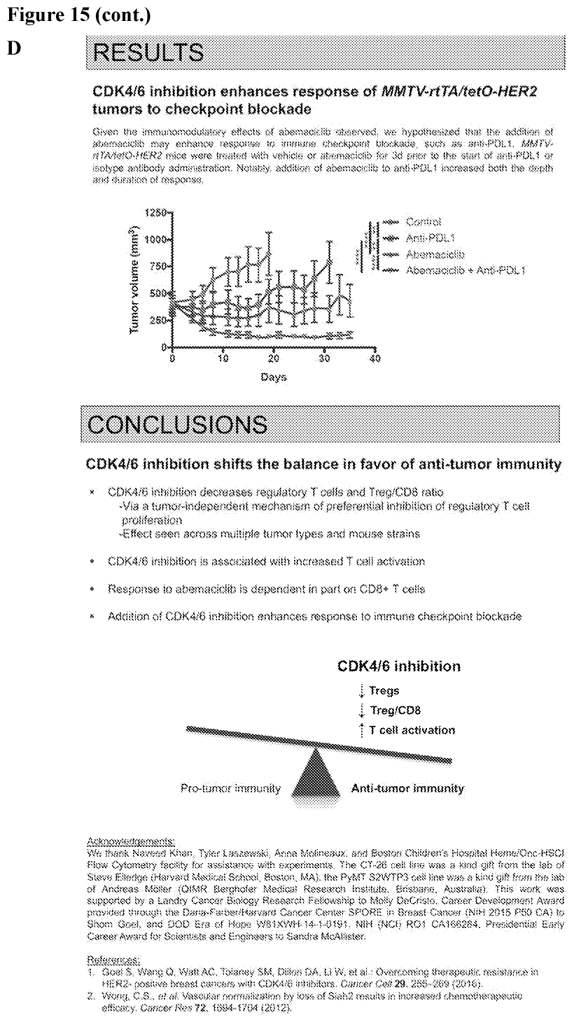

Based on the foregoing, although CDK4/6 inhibitors are thought to exert their primary anti-tumor effects by inducing cancer cell cycle arrest, here, using a recently described and clinically relevant transgenic model of breast cancer (Goel et al. (2016), supra), a previously unidentified CDK4/6 inhibitor function, i.e., the induction of an anti-tumor immune response, was revealed herein. This immune response arises from the combination of two phenomena: enhanced antigen presentation by tumor cells and a re-programming of the immune suppressive microenvironment (FIG. 13G and data summarized in FIG. 15).

Tumors evade the immune system through several mechanisms, including impaired antigen presentation. Indeed, defects in the interferon signaling pathway and downstream transcription factors foster immune evasion and resistance to immune checkpoint blockade (Gao et al. (2016) Cell 167:397-404; Zaretsky et al. (2016) N Engl J Med 375:819-829; Yoshihama et al. (2016) Proc Natl Acad Sci USA 113:5999-6004). It is shown herein that CDK4/6 inhibition markedly reduces expression of the E2F target gene DNMT1, which is associated with increased expression of endogenous retroviral genes (ERVs), upregulation of the double-stranded RNA response, and production of type III interferons. Such “viral mimicry” in tumor cells has been described after direct inhibition of DNA methyltransferases (Roulois et al. (2015), supra and Chiappinelli et al. (2015), supra), resulting in activating ISG expressions and enhancing tumor cell antigen presentation and tumor immunogenicity (FIG. 13G). In support of these studies, an expression signature reflecting a viral defense program has been found to correlate with duration of response to immune checkpoint blockade in melanoma patients (Chiappinelli et al. (2015), supra). CDK4/6 inhibitors inhibit tumor proliferation and promotes regression by inhibiting DNMT1.

Prior to these studies, effects of CDK4/6 inhibitors on the tumor immune microenvironment were unknown. Indeed, concerns were raised that these agents might render immune checkpoint therapy less effective due to T cell cycle inhibition (Sherr (2016) N Engl J Med 375:1920-1923). However, the results provided herein demonstrate that the pleiotropic effects of CDK4/6 inhibitors on the immune microenvironment, i.e., heightened antigen presentation, increased T cell numbers, a reduction in T cell exhaustion, and diminution of Tregs through preferential inhibition of proliferation (FIG. 13G), confirm their function of anti-tumor immunity.

Approximately 70 percent of human breast cancers are estrogen receptor-positive, and most of these classify as having a “luminal” pattern of gene expression (Parker et al. (2009) J Clin Oncol 27:1160-1167). Luminal tumors usually retain Rb expression and show the greatest clinical response to CDK4/6 inhibitors (Patnaik et al. (2016), supra; Finn et al. (2009), supra). Importantly, tumors with CCND1 amplification in patients display reduced expression of MHC class I molecules when compared to non-amplified tumors. Furthermore, high levels of Tregs in luminal tumors specifically predict a poor clinical outcome (Bates et al. (2006) J Clin Oncol 24:5373-5380). Therefore, CDK4/6 inhibitors are believed to affect two important mechanisms of immune evasion within luminal breast tumors. While these cancers generally have very low response rates to immune-based therapies, CDK4/6 inhibitors are capable of enhancing their susceptibility to immune checkpoint blockade (e.g., an anti-PD-L1 antibody used in the current studies) by converting them from being immunologically “cold” to immunologically “hot.”

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web and/or the National Center for Biotechnology Information (NCBI) on the World Wide Web.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of selectively reducing the number of circulating regulatory T cells (Tregs) in a subject, comprising administering to the subject a therapeutically effective amount of at least one agent that selectively inhibits or blocks the expression or activity of CDK4 and/or CDK6 such that the number of Tregs in the subject is selectively reduced.

2. The method of claim 1, wherein the Tregs comprise CD4+CD25+, CD4+FOXP3+, and/or CD4+CD25+FOXP3+ Tregs.

3. The method of claim 1 or 2, wherein the at least one agent significantly reduces the number of the Tregs in the spleen of the subject.

4. The method of any one of claims 1-3, wherein the at least one agent significantly reduces the number of the Tregs in the lymph nodes of the subject.

5. The method of any one of claims 1-4, wherein the at least one agent does not significantly affect differentiation of naïve CD4+ T cells into Tregs in the subject.

6. The method of any one of claims 1-5, wherein the at least one agent does not significantly affect Treg apoptosis in the subject.

7. The method of any one of claims 1-6, wherein the at least one agent does not significantly change the cell number of at least one cell type selected from the group consisting of B lymphocytes, natural killer cells, neutrophils, and monocytes.

8. The method of any one of claims 1-7, wherein the at least one agent reduces the ratio of Tregs to CD3+ T cells and/or the ratio of Tregs to CD8+ T cells in the subject.

9. The method of any one of claims 1-8, wherein the at least one agent does not significantly modulate the number of CD8+ T cells and/or CD4+CD25− T cells.

10. The method of any one of claims 1-9, wherein the at least one agent reduces the expression of at least one marker selected from the group consisting of PD-1, TIM-3, CTLA-4, and LAG3 on the surface of CD4+ and/or CD8+ T cells.

11. The method of any one of claims 1-10, wherein the at least one agent increases antigen presentation in the subject.

12. The method of any one of claims 1-11, wherein the at least one agent increases MHC class I expression in the subject.

13. The method of any one of claims 1-12, wherein the at least one agent increases T cell-mediated cytotoxicity in the subject.

14. The method of any one of claims 1-13, wherein the at least one agent increases interferon production, signaling, and/or secretion in the subject.

15. The method of claim 14, wherein the at least one agent increases type III interferon production in the subject.

16. The method of claim 14, wherein the at least one agent increases expression of at least one gene selected from the group consisting of STAT1, STAT2, IRF2, IRF6, IRF7, IRF9, NLRC5, OAS1, OAS2, IFIT1, IFIT2, IFIT6, BST2, SP100, RSAD2, CXCL9, CXCL10, CXCL11, Icam1, Vcam1, IL-29, IL-28a, IL-28b, ERV3-1, ERVK13-1, RIG-1, LGP2, and MDA5 in the subject.

17. The method of any one of claims 1-16, wherein the at least one agent inhibits at least one DNA methyltransferase (DNMT) in the subject.

18. The method of claim 17, wherein the at least one agent inhibits DNMT1 expression in the subject.

19. The method of any one of claims 1-18, wherein the at least one agent does not significantly enhance senescence associated secretory phenotype (SASP) in the subject.

20. The method of any one of claims 1-19, wherein the at least one agent is selected from the group consisting of: a small molecule CDK4 antagonist, a blocking intrabody or antibody that binds CDK4, a non-activating form of CDK4, a soluble form of an CDK4 natural binding partner, a CDK4 fusion protein, a nucleic acid molecule that blocks CDK4 transcription or translation, a small molecule CDK6 antagonist, a blocking intrabody or antibody that recognizes CDK6, a non-activating form of CDK6, a soluble form of a CDK6 natural binding partner, a CDK6 fusion protein, and a nucleic acid molecule that blocks CDK6 transcription or translation.

21. The method of any one of claims 1-20, wherein said at least one agent comprises a small molecule that inhibits or blocks CDK4 and/or CDK6 expression or activity.

22. The method of claim 21, wherein said small molecule is selected from the group consisting of abemaciclib, palbociclib, and ribociclib.

23. The method of any one of preceding claim, wherein said at least one agent comprises an RNA interfering agent which inhibits or blocks CDK4 and/or CDK6 expression or activity.

24. The method of claim 23, wherein said RNA interfering agent is a small interfering RNA (siRNA), small hairpin RNA (shRNA), microRNA (miRNA), or a piwiRNA (piRNA).

25. The method of any one of claims 1-20, wherein said at least one agent comprises an antisense oligonucleotide complementary to CDK4 and/or CDK6.

26. The method of any one of claims 1-20, wherein said at least one agent comprises a peptide or peptidomimetic that inhibits or blocks CDK4 and/or CDK6 expression or activity.

27. The method of any one of claims 1-20, wherein said at least one agent comprises an aptamer that inhibits or blocks CDK4 and/or CDK6 expression or activity.

28. The method of any one of claims 1-20, wherein said at least one agent is an intrabody or antibody, or an antigen binding fragment thereof, which specifically binds to CDK4 and/or CDK6.

29. The method of claim 28, wherein said intrabody or antibody, or antigen binding fragment thereof, is murine, chimeric, humanized, or human.

30. The method of claim 28 or 29, wherein said intrabody or antibody, or antigen binding fragment thereof, is detectably labeled, comprises an effector domain, comprises an Fc domain, and/or is selected from the group consisting of Fv, F(ab′)2, Fab′, dsFv, scFv, sc(Fv)2, and diabody fragments.

31. The method of any one of claims 1-30, wherein said at least one agent is administered in a pharmaceutically acceptable formulation.

32. The method of any one of claims 1-31, wherein the subject has a condition that would benefit from upregulation of an immune response.

33. The method of claim 32, wherein the subject has a condition selected from the group consisting of a cancer, a viral infection, a bacterial infection, a protozoal infection, a helminth infection, asthma associated with impaired airway tolerance, and an immunosuppressive disease.

34. The method of claim 33, wherein the condition is a cancer.

35. The method of claim 34, wherein the cancer is breast cancer and/or a colorectal cancer.

36. The method of any one of claims 1-35, wherein at least some of the subject's immune cells, Tregs, or cancer cells express Rb and/or has functional Rb signaling.

37. The method of any one of claims 1-35, wherein at least some of the subject's immune cells, Tregs, or cancer cells have defective Rb expression and/or defective Rb signaling.

38. The method of claim 37, wherein at least some of the subject's Tregs or cancer cells harbor genomic mutations causing defective Rb expression and/or defective Rb signaling.

39. The method of any one of claims 33-38, wherein the condition is resistant to immune checkpoint blockade.

40. The method of claim 39, wherein the at least one agent increases the susceptibility to immune checkpoint blockade of the subject's cells, immune cells, Tregs, or cancer cells in the subject.

41. The method of any one of claims 33-40, wherein at least one agent:
a) increases the number of cancer infiltrating CD3+ T cells in the subject;
b) increases antigen presentation by cancer cells in the subject;
c) increases MHC class I expression by cancer cells in the subject;
d) increases interferon production, signaling, and/or secretion by cancer cells in the subject;
e) increases type III interferon production, signaling, and/or secretion by cancer cells in the subject;
f) increases expression of at least one gene selected from the group consisting of STAT1, STAT2, IRF2, IRF6, IRF7, IRF9, NLRC5, OAS1, OAS2, IFIT1, IFIT2, IFIT6, BST2, SP100, RSAD2, CXCL9, CXCL10, CXCL11, Icam1, Vcam1, IL-29, IL-28a, IL-28b, ERV3-1, ERVK13-1, RIG-1, LGP2, and MDA5 by cancer cells in the subject;
g) inhibits expression of at least one DNA methyltransferase (DNMT) by cancer cells in the subject; and/or
h) inhibits expression of DNMT1 expression by cancer cells in the subject.

42. The method of any one of claims 1-41, further comprising administering one or more additional agents or therapies that upregulates an immune response.

43. The method of claim 42, wherein the one or more additional agents or therapies is selected from the group consisting of immunotherapy, a vaccine, chemotherapy, radiation, epigenetic modifiers, and targeted therapy.

44. The method of claim 43, wherein the immunotherapy is selected from the group consisting of immune checkpoint inhibitor therapy, a sensitized antigen presenting cell, an oncolytic virus, an expression vector comprising an anticancer gene, and an inhibitor of a cancer antigen or a disease antigen.

45. The method of claim 44, wherein the immune checkpoint inhibitor therapy comprises reducing or inhibiting the expression and/or function of an immune checkpoint molecule selected from the group consisting of CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR in the subject.

46. The method of claim 45, wherein the immune checkpoint inhibitor therapy targets an immune checkpoint selected from the group consisting of PD-1, CTLA-4, PD-L1, PD-L2, and combinations thereof.

47. The method of any one of claims 42-46, wherein the at least one agent is administered prior to administering the one or more additional agents or therapies that upregulates the immune response, optionally wherein the at least one agent is preadministered before subsequent administration of a combination of the at least one agent and the one or more additional agents or therapies that upregulates the immune response.

48. The method of any one of claims 1-47, wherein the subject is a mammal.

49. The method of claim 48, wherein the mammal is an animal model of the condition.

50. The method of claim 48, wherein the mammal is a human.

51. A method of upregulating an immune response in a subject in need thereof, comprising administering to the subject a combination of i) a therapeutically effective amount of at least one agent that selectively inhibits or blocks the expression or activity of both CDK4 and/or CDK6, and ii) an immunotherapy, such that an immune response is upregulated in the subject.

52. The method of claim 51, wherein the subject has a condition selected from the group consisting of a cancer, a viral infection, a bacterial infection, a protozoal infection, a helminth infection, asthma associated with impaired airway tolerance, and an immunosuppressive disease.

53. The method of claim 52, wherein the condition is a cancer.

54. The method of claim 53, wherein the cancer is breast cancer and/or a colorectal cancer.

55. The method of any one of claims 51-54, wherein at least some of the subject's immune cells, Tregs, or cancer cells express Rb and/or has functional Rb signaling.

56. The method of any one of claims 51-54, wherein at least some of the subject's immune cells, Tregs, or cancer cells have defective Rb expression and/or defective Rb signaling.

57. The method of claim 56, wherein at least some of the subject's Tregs or cancer cells harbor genomic mutations causing defective Rb expression and/or defective Rb signaling.

58. The method of any one of claims 52-57, wherein the condition is resistant to immune checkpoint blockade.

59. The method of claim 58, wherein the at least one agent increases the susceptibility to immune checkpoint blockade of the subject's cells, immune cells, Tregs, or cancer cells in the subject.

60. The method of any one of claims 51-59, wherein the at least one agent:
a) increases the number of cancer infiltrating CD3+ T cells in the subject;
b) increases antigen presentation by cancer cells in the subject;
c) increases MHC class I expression by cancer cells in the subject;
d) increases interferon production, signaling, and/or secretion by cancer cells in the subject;
e) increases type III interferon production, signaling, and/or secretion by cancer cells in the subject;
f) increases expression of at least one gene selected from the group consisting of STAT1, STAT2, IRF2, IRF6, IRF7, IRF9, NLRC5, OAS1, OAS2, IFIT1, IFIT2, IFIT6, BST2, SP100, RSAD2, CXCL9, CXCL10, CXCL11, Icam1, Vcam1, IL-29, IL-28a, IL-28b, ERV3-1, ERVK13-1, RIG-1, LGP2, and MDA5 by cancer cells in the subject;
g) inhibits expression of at least one DNA methyltransferase (DNMT) by cancer cells in the subject; and/or
h) inhibits expression of DNMT1 expression by cancer cells in the subject.

61. The method of any one of claims 51-60, wherein the at least one agent is administered prior to administering the immunotherapy, optionally wherein the at least one agent is preadministered before subsequent administration of a combination of the at least one agent and the immunotherapy.

62. The method of any one of claims 51-61, further comprising administering one or more additional agents or therapies that upregulates an immune response.

63. The method of claim 62, wherein the one or more additional agents or therapies is selected from the group consisting of a vaccine, chemotherapy, radiation, epigenetic modifiers, and targeted therapy.

64. The method of claim 51, wherein the immunotherapy is selected from the group consisting of immune checkpoint inhibitor therapy, a sensitized antigen presenting cell, an oncolytic virus, an expression vector comprising an anticancer gene, and an inhibitor of a cancer antigen or a disease antigen.

65. The method of claim 64, wherein the immune checkpoint inhibitor therapy comprises reducing or inhibiting the expression and/or function of an immune checkpoint molecule selected from the group consisting of CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR in the subject.

66. The method of claim 65, wherein the immune checkpoint inhibitor therapy targets an immune checkpoint selected from the group consisting of PD-1, CTLA-4, PD-L1, PD-L2, and combinations thereof.

67. The method of any one of claims 51-66, wherein the at least one agent significantly reduces the number of Tregs in the spleen of the subject.

68. The method of any one of claims 51-67, wherein the at least one agent significantly reduce the number of Tregs in the lymph nodes of the subject.

69. The method of any one of claims 51-68, wherein the at least one agent does not significantly affect differentiation of naïve CD4+ T cells into Tregs in the subject.

70. The method of any one of claims 51-69, wherein the at least one agent does not significantly affect Treg apoptosis in the subject.

71. The method of any one of claims 51-70, wherein the at least one agent does not significantly change the cell number of at least one cell type selected from the group consisting of B lymphocytes, natural killer cells, neutrophils, and monocytes.

72. The method of any one of claims 51-71, wherein the at least one agent reduces the ratio of Tregs to CD3+ T cells and/or the ratio of Tregs to CD8+ T cells in the subject.

73. The method of any one of claims 67-72, wherein the Tregs comprise CD4+CD25+, CD4+FOXP3+, and/or CD4+CD25+FOXP3+ Tregs.

74. The method of any one of claims 51-73, wherein the at least one agent does not significantly modulate the number of CD8+ T cells and/or CD4+CD25− T cells.

75. The method of any one of claims 51-74, wherein the at least one agent reduces the expression of at least one marker selected from the group consisting of PD-1, TIM-3, CTLA-4, and LAG3 on the surface of CD4+ and/or CD8+ T cells.

76. The method of any one of claims 51-75, wherein the at least one agent increases antigen presentation in the subject.

77. The method of any one of claims 51-76, wherein the at least one agent increases MHC class I expression in the subject.

78. The method of any one of claims 51-77, wherein the at least one agent increases T cell-mediated cytotoxicity in the subject.

79. The method of any one of claims 51-78, wherein the at least one agent increases interferon production, signaling, and/or secretion in the subject.

80. The method of claim 79, wherein the at least one agent increases type III interferon production in the subject.

81. The method of claim 80, wherein the at least one agent increases expression of at least one gene selected from the group consisting of STAT1, STAT2, IRF2, IRF6, IRF7, IRF9, NLRC5, OAS1, OAS2, IFIT1, IFIT2, IFIT6, BST2, SP100, RSAD2, CXCL9, CXCL10, CXCL11, Icam1, Vcam1, IL-29, IL-28a, IL-28b, ERV3-1, ERVK13-1, RIG-1, LGP2, and MDA5 in the subject.

82. The method of any one of claims 51-81, wherein the at least one agent inhibits at least one DNA methyltransferase (DNMT) in the subject.

83. The method of claim 82, wherein the at least one agent inhibits DNMT1 expression in the subject.

84. The method of any one of claims 51-83, wherein the at least one agent does not significantly enhance senescence associated secretory phenotype (SASP) in the subject.

85. The method of any one of claims 51-84, wherein the at least one agent is selected from the group consisting of: a small molecule CDK4 antagonist, a blocking intrabody or antibody that binds CDK4, a non-activating form of CDK4, a soluble form of an CDK4 natural binding partner, a CDK4 fusion protein, a nucleic acid molecule that blocks CDK4 transcription or translation, a small molecule CDK6 antagonist, a blocking intrabody or antibody that recognizes CDK6, a non-activating form of CDK6, a soluble form of a CDK6 natural binding partner, a CDK6 fusion protein, and a nucleic acid molecule that blocks CDK6 transcription or translation.

86. The method of any one of claims 51-85, wherein said at least one agent comprises a small molecule that inhibits or blocks CDK4 and/or CDK6 expression or activity.

87. The method of claim 86, wherein said small molecule is selected from the group consisting of abemaciclib, palbociclib, and ribociclib.

88. The method of any one of claims 51-85, wherein said at least one agent comprises an RNA interfering agent which inhibits or blocks CDK4 and/or CDK6 expression or activity.

89. The method of claim 88, wherein said RNA interfering agent is a small interfering RNA (siRNA), small hairpin RNA (shRNA), microRNA (miRNA), or a piwiRNA (piRNA).

90. The method of any one of claims 51-85, wherein said at least one agent comprises an antisense oligonucleotide complementary to CDK4 and/or CDK6.

91. The method of any one of claims 51-85, wherein said at least one agent comprises a peptide or peptidomimetic that inhibits or blocks CDK4 and/or CDK6 expression or activity.

92. The method of any one of claims 51-85, wherein said at least one agent comprises an aptamer that inhibits or blocks CDK4 and/or CDK6 expression or activity.

93. The method of any one of claims 51-85, wherein said at least one agent is an intrabody or antibody, or an antigen binding fragment thereof, which specifically binds to CDK4 and/or CDK6.

94. The method of claim 93, wherein said intrabody or antibody, or antigen binding fragment thereof, is murine, chimeric, humanized, or human.

95. The method of claim 93 or 94, wherein said intrabody or antibody, or antigen binding fragment thereof, is detectably labeled, comprises an effector domain, comprises an Fc domain, and/or is selected from the group consisting of Fv, F(ab′)2, Fab′, dsFv, scFv, sc(Fv)2, and diabody fragments.

96. The method of any one of claims 51-95, wherein said at least one agent is administered in a pharmaceutically acceptable formulation.

97. The method of any one of claims 51-96, wherein the subject is a mammal.

98. The method of claim 97, wherein the mammal is an animal model of the condition.

99. The method of claim 99, wherein the mammal is a human.


SEQUENCE LISTING (35 sequences)

SEQ ID NO: 1 - DNA, 2020 bp, Homo sapiens
cacctcctgtccgcccctcagcgcatgggtggcggtcacgtgcccagaacgtccggcgttcgccccgccctcccagtttccgcgcgcctctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaataatggtcttttttttttttttaatgtttcttcctctgtttggctttgccattgtgcgatttggaaaaaccacttggaagaagggactttcctgcaaaaccttaaagactggttaaattacagggcctaggaagtcagtggagccccttgactgacaaagcttagaaaggaactgaaattgcttctttgaatatggattttaggcggggcgtggtggctcacgcctataatcccagcacgttgggaggccaacgcgggtggatcacctgaggtcaggagttcgagaccagcctgactaacatggtgaaaccctgtctctactaaaaatacaaaattagtcaggcgtggtggtgcacacctgtaatcccagctacttgggagactgaggcaggaggatcgcttgaacccgggaggcagaggttgcggtgagccgagatcatgccattgcactccagcctgggcaacagagcaagactctgtgtcaaaaaaaaaaaaagaatatagatttttaaatggcaaaaaaaaaaaaaaaaaa

SEQ ID NO: 2 - protein, 303 aa, Homo sapiens
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 3 - DNA, 1365 bp, Mus musculus
gtgggggtgagggggcctctctagctcgcggcctgtgtctatggtctggcccgaagcgtccagctgcccgggaccgatccccggtgtatggcgccgcaggaaccggctcccgggcccagataaagggccacctccagagctcttagccgagcgtaagatcccctgcttcgagaatggctgccactcgatatgaacccgtggctgaaattggtgtcggtgcctatgggacggtgtacaaagcccgagatccccacagtggccactttgtggccctcaagagtgtgagagttcctaatggaggagcagctggagggggccttcccgtcagcacagttcgtgaggtggccttgttaaggaggctggaggcctttgaacatcccaatgttgtacggctgatggatgtctgtgctacttcccgaactgatcgggacatcaaggtcaccctagtgtttgagcatatagaccaggacctgaggacatacctggacaaagcacctccaccgggcctgccggttgagaccattaaggatctaatgcgtcagtttctaagcggcctggattttcttcatgcaaactgcattgttcaccgggacctgaagccagagaacattctagtgacaagtaatgggaccgtcaagctggctgactttggcctagctagaatctacagctaccagatggccctcacgcctgtggtggttacgctctggtaccgagctcctgaagttcttctgcagtctacatacgcaacacccgtggacatgtggagcgttggctgtatctttgcagagatgttccgtcggaagcctctcttctgtggaaactctgaagccgaccagttggggaaaatctttgatctcattggattgcctccagaagacgactggcctcgagaggtatctctacctcgaggagcctttgcccccagagggcctcggccagtgcagtcagtggtgccagagatggaggagtctggagcgcagctgctactggaaatgctgacctttaacccacataagcgaatctctgccttccgagccctgcagcactcctacctgcacaaggaggaaagcgacgcagagtgagaagaggggctgcctttcccagtcttggtggagaaaccctcgctgaagcggcagcctctgtttccccccaaggctgtggagaatcctccagttttttacagagaatattttaagccttaaataacaagtccccacctctccttacgaggttcacccccattaccctcccctagctctacactaaagggcaggtgtatctgtcttcttccctccctgatttatactgggatcttttttatacaggaaaacaagacaagacaaaaaaaaaaaaaaaaaaaaa

SEQ ID NO: 4 - protein, 303 aa, Mus musculus
MAATRYEPVAEIGVGAYGTVYKARDPHSGHFVALKSVRVPNGGAAGGGLPVSTVREVALLRRLEAFEHPNVVRLMDVCATSRTDRDIKVTLVFEHIDQDLRTYLDKAPPPGLPVETIKDLMRQFLSGLDFLHANCIVHRDLKPENILVTSNGTVKLADFGLARIYSYQMALTPVVVTLWYRAPEVLLQSTYATPVDMWSVGCIFAEMFRRKPLFCGNSEADQLGKIFDLIGLPPEDDWPREVSLPRGAFAPRGPRPVQSVVPEMEESGAQLLLEMLTFNPHKRISAFRALQHSYLHKEESDAE

SEQ ID NO: 5 - DNA, 11628 bp, Homo sapiens
aacctctccgcgcgaagacggcttcagccctgcagggaaagaaaagtgcaatgattctggactgagacgcgcttgggcagaggctatgtaatcgtgtctgtgttgaggacttcgcttcgaggagggaagaggagggatcggctcgctcctccggcggcggcggcggcggcgactctgcaggcggagtttcgcggcggcggcaccagggttacgccagccccgcggggaggtctctccatccagcttctgcagcggcgaaagccccagcgcccgagcgcctgagccggcggggagcaagtaaagctagaccgatctccggggagccccggagtaggcgagcggcggccgccagctagttgagcgcaccccccgcccgccccagcggcgccgcggcgggcggcgtccaggcggcatggagaaggacggcctgtgccgcgctgaccagcagtacgaatgcgtggcggagatcggggagggcgcctatgggaaggtgttcaaggcccgcgacttgaagaacggaggccgtttcgtggcgttgaagcgcgtgcgggtgcagaccggcgaggagggcatgccgctctccaccatccgcgaggtggcggtgctgaggcacctggagaccttcgagcaccccaacgtggtcaggttgtttgatgtgtgcacagtgtcacgaacagacagagaaaccaaactaactttagtgtttgaacatgtcgatcaagacttgaccacttacttggataaagttccagagcctggagtgcccactgaaaccataaaggatatgatgtttcagcttctccgaggtctggactttcttcattcacaccgagtagtgcatcgcgatctaaaaccacagaacattctggtgaccagcagcggacaaataaaactcgctgacttcggccttgcccgcatctatagtttccagatggctctaacctcagtggtcgtcacgctgtggtacagagcacccgaagtcttgctccagtccagctacgccacccccgtggatctctggagtgttggctgcatatttgcagaaatgtttcgtagaaagcctctttttcgtggaagttcagatgttgatcaactaggaaaaatcttggacgtgattggactcccaggagaagaagactggcctagagatgttgcccttcccaggcaggcttttcattcaaaatctgcccaaccaattgagaagtttgtaacagatatcgatgaactaggcaaagacctacttctgaagtgtttgacatttaacccagccaaaagaatatctgcctacagtgccctgtctcacccatacttccaggacctggaaaggtgcaaagaaaacctggattcccacctgccgcccagccagaacacctcggagctgaatacagcctgaggcctcagcagccgccttaagctgatcctgcggagaacacccttggtggcttatgggtccccctcagcaagccctacagagctgtggaggattgctatctggaggccttccagctgctgtcttctggacaggctctgcttctccaaggaaaccgcctagtttactgttttgaaatcaatgcaagagtgattgcagctttatgttcatttgtttgtttgtttgtctgtttgtttcaagaacctggaaaaattccagaagaagagaagctgctgaccaattgtgctgccatttgatttttctaaccttgaatgctgccagtgtggagtgggtaatccaggcacagctgagttatgatgtaatctctctgcagctgccgggcctgatttggtacttttgagtgtgtgtgtgcatgtgtgtgtgtgtgtgtgtgtgtgtgtgtgtgtatgtgagagattctgtgatcttttaaagtgttactttttgtaaacgacaagaataattcaattttaaagactcaaggtggtcagtaaataacaggcatttgttcactgaaggtgattcaccaaaatagtcttctcaaattagaaagttaaccccatgtcctcagcatttcttttctggccaaaagcagtaaatttgctagcagtaaaagatgaagttttatacacacagcaaaaaggagaaaaaattctagtatattttaagagatgtgcatgcattctatttagtcttcagaatgctgaatttacttgttgtaagtctattttaaccttctgtatgacatcatgctttatcatttcttttggaaaatagcctgtaagctttttattacttgctataggtttagggagtgtacctcagatagattttaaaaaaaagaatagaaagcctttatttcctggtttgaaattcctttcttccctttttttgttgttgttattgttgtttgttgttgttattttgtttttgtttttaggaatttgtcagaaactctttcctgttttggtttggagagtagttctctctaactagagacaggagtggccttgaaattttcctcatctattacactgtactttctgccacacactgccttgttggcaaagtatccatcttgtctatctcccggcacttctgaaatatattgctaccattgtataactaataacagattgcttaagctgttcccatgcaccacctgtttgcttgctttcaatgaacctttcataaattcgcagtctcagcttatggtttatggcctcgattctgcaaacctaacagggtcacatatgttctctaatgcagtccttctacctggtgtttacttttgttacctaaataatgagtaggatcttgttttgttttatcaccagcacacagattgctataaactgttactttgtgaattacatttttatagaagatattttcagtgtctttacctgagggtatgtctttagctatgttttagggccatacatttactctatcaaatgatcttttctccatcccccaggctgtgcttatttctagtgccttgtgctcactcctgctctctacagagccagcctggcctgggcattgtaaacagcttttcctttttctcttactgttttctctacagtcctttatatttcataccatctctgccttataagtggtttagtgctcagttggctctagtaaccagaggacacagaaagtatcttttggaaagtttagccacctgtgctttctgactcagagtgcatgcaacagttagatcatgcaacagttagattatgtttagggttaggattttcaaagaatggaggttgctgcactcagaaaataattcagatcatgtttatgcattattaagttgtactgaattctttgcagcttaatgtgatatatgactatcttgaacaagagaaaaaactaggagatgtttctcctgaagagcttttggggttgggaactattcttttttaattgctgtactacttaacattgttctaattcagtagcttgaggaacaggaacattgttttctagagcaagataataaaggagatgggccatacaaatgttttctactttcgttgtgacaacattgattaggtgttgtcagtactataaatgcttgagatataatgaatccacagcattcaaggtcaggtctactcaaagtctcacatggaaaagtgagttctgcctttcctttgatcgagggtcaaaatacaaagacatttttgctagggcctacaaattgaatttaaaaactcactgcactgattcatctgagctttttggttagtattcatggctagagtgaacatagctttagtttttgctgttgtaaaagtgttttcataagttcactcaagaaaaatgcagctgttctgaactggaatttttcagcattctttagaattttaaatgagtagagagctcaacttttattcctagcatctgcttttgactcatttctaggcagtgcttatgaagaaaaattaaagcacaaacattctggcattcaatcgttggcagattatcttctgatgacacagaatgaaagggcatctcagcctctctgaactttgtaaaaatctgtccccagttcttccatcggtgtagttgttgcatttgagtgaatactctcttgatttatgtattttatgtccagattcgccatttctgaaatccagatccaacacaagcagtcttgccgttagggcattttgaagcagatagtagagtaagaacttagtgactacagcttattcttctgtaacatatggtttcaaacatctttgccaaaagctaagcagtggtgaactgaaaagggcatattgccccaaggttacactgaagcagctcatagcaagttaaaatattgtgacagatttgaaatcatgtttgaatttcatagtaggaccagtacaagaatgtccctgctagtttctgtttgatgtttggttctggcggctcaggcattttgggaactgttgcacagggtggagtcaaaacaacctacatataaaaagagaaaaagagaaacttgtccatttagctttcataagaaatcccatggcaaagggtaataaaaaggacctaatcttaaaaatacaatttctaagcacttgtaagaacccagtgggttggagcctcccactttgtccctcctttgaagtggatgggaactcaaggtgcaaagaacctgttttggaagaaagcttggggccatttcagccccctgtattctcatgattttctctcaggaagcacacactgtgaatggcagacttttcatttagccccaggtgacttactaaaaatagttgaaaattattcacctaagaatagaatctcagcattgtgttaaataaaaatgaaagctttagaaggcatgagatgttcctatcttaaataaagcatgtttcttttctatagagaaatgtatagtttgactctccagaatgtactatccatcttgatgagaaaactcttaaatagtaccaaacattttgaactttaaattatgtatttaaagtgagtgtttaagaaactgtagctgcttcttttacaagtggtgcctattaaagtcagtaatggccattattgttccattgtggaaattaaattatgtaagcttcctaatatcataaacatattaaaattcttctaaaatattgcttttcttttaagtgacaatttgactattcttatgataagcacatgagagtgtcttacattttccaaaagcaggctttaattgcatagttgagtctaggaaaaaataatgttaaaagtgaatatgccaccataattacttaattatgttagtatagaaactacagaatatttaccctggaaagaaaatattggaatgttattataaactcttagatatttatataattcaaaagaatgcatgtttcacattgtgacagataaagatgtatgatttctaaggctttaaaaattattcataaaacagtgggcaatagataaaggaaattctggagaaaatgaaggtatttaaagggtagtttcaaagctatatatattttgaaggatatattctttatgaacaaatatattgtaaaaatttatactaaggtcatctggtaactgtgggattaatatggtcgaaaacaaatgttatggagaagctgtcccaagcaaactaaattacctgtacttttttcccatttcaagggaagaggcaaccacatgaagcaatacttcttacacatgcctaagaacgttcattgaaaaaataaatttttaaaaggcatgtgtttcctatgccaccaatacttttgaaaaattgtgaaccttacccaaaaccatttatcatgtccattaagtatatttgggtatataattaggaagatatttacatgttccatctccacagtggaaaaacttattgaggctaccaaagtgtgccaagaaatgtaagtccttagagtaattagaaatgctgttttcctcaaaagcatgagaaactagcattttcatttcttatttactccctttctatatcaatgcaattcacaacccaattttaatacatccctatatctcaagcatttctatcttgtactttttcagaaaataaaccaaaaataatcctttggtctctctatcttctgacctttgtaagcaacagaaatgtaaaaacagaaggggtccaatttttacacgtttttttctcaagtagcctttctggggatttttattttcttaatgaagtgccaatcagcttttcaaaatgttttctatttctcagcatttccaggaagtgataacgtttagctaaatgagtagaagtggacttccttcaacatattgttaccttgtctagccttaggaagaaaacaagagccacctgaaaataaatacaggctcttttcgagcatctgctgaaatactgttacagcaatttgaagttgatgtggtaggaaaggaaggtgacttttcttgcaaaagtctttctaaacattcacactgtcctaagagatgagctttcttgttttattccggtatattccacaaggtggcacttttagagaaaaacaaatctgatgaagactaaagaggtacttctaaaagagatttcattctaactttatttttctgcgcatatttaactctttcctagcacttgttttttgggatgattaatagtctctataatgttctgtaacttcaatattttacttgttacctaggttctgaacaattgtctgcaaataaattgttcttaaggatggataatacacccattttgatcatttaagtaaagaaagcctagtcattcattcagtcaagaaaaaatttttgaagtacccagttaccttacttttctagattaaaacaggcttagttactaaaaaggcagtcctcatctgtgaacaggatagtttcgttagaagtataaaactcctttagtggccccagttaaaacacacataccctctctgctgctttcaaattccctagcatggtggcctttcaacattgattaaattttaaaatcctaatttaaagatcaggtgagcaaaatgagtagcacatcagtaattcagtagacaaaacttttgtctgaaaaattgctgtattgaaacagagccctaaaataccaaaagaccaggtaattttaacatttgtggaatcacaaatgtaaattcataagaagctctaattaaaaaaaaaaagtctgaagtatatgagcataacaacttaggagtgtgtctacatacttaacttttgaagttttttggcaactttatatactttttttaaatttacaagtctacttaaagacttcttataccccaaatgattaagttaattttagaggtcacctttctcacagcagtgtcacttgaaatttagtagggaaggatattgcagtatttttcagtttccttagcacagcaccacagaaagcagcttattccttttgagtggcagacactcgacggtgcctgcccaactttcctcctgagtggcaagcagatgagtctcagtaattcatactgaaccaaaatgccacatacactaggggcagtcagaaactggctgagaaatcccccgcctcattcgcccctctgctcccaggaactagagtccagttaaagcccctatgcgaaaggccgaattccaccccagggtttgttataacagtggccagtctgaaccccatttgctcgtgctcaaaacttgattcccacttgaaagccttccgggcgcgctgcctcgttgccccgcccctttggcaggagagaggcagtgggcgaggccgggctggggccccgcctcccactcacctgccggtgcctgaaattatgtgcggccccgcgggctgctttccgaggtcagagtgccctgctgctgtctcagaggcatctgttctgcaaatcttaggaagaaaaatgtccctagtagcaaacgggtgtcttctgtgcataaataagtacaacacaattctccgaaagttcgggtaaaaagagatgcggtagcagctgccctgtgtgaagctgtctaccccgcatctctcaggcgctaagctcagtttttgtttttgtttttgtttttttaaagaaaagatgtataattgcaggaatttttttttatttttttattttccatcattctatatatgtgatggtgaaagatatgcctggaaaagttttgttttgaaaagtttattttctgcttcgtcttcagttggcaaaagctctcaattctttagcttccagtttcttttctctctttttctttgttaggtaattaaaggtatgtaaacaaattatctcatgtagcaggggattttcatgttgagaggaatcttccgtgtgagttgtttggtcacacaaataaccctttctcaattttaggagtttggattgtcaaatgtaggtttttctcaaagggggcatataactacatattgactgccaagaactatgactgtagcactaatcagcacacatagagccacacaattatttaatttctaactctctgtggtccctagaaaaattccgttgatgtgcttaggttaaagttctgaagatacccgttgtacccttacttgaaagtttctaatcttaagttttatgaaatgcaataatatgtatcagctagcaatatttctgtgatcaccaacaactctcagtttgatcttaaagtctgaataataaaacaaatcccagcagtaatacatttcttaaacctcacagtgcatgatatatcttttcattctgatcctgtgtttgcaaaaatatacacatgtatatcatagttcctcactttttattcatttgttttcctattacctgtagtaaatatattagttagtacatggaatttatagcatcagctacccccaggaacagcacctgacaggcgggggattttttttcaagttgttctacatttgcataaattatttctattattattcatgtatgttatttatttctgaatcacactagtcctgtgaaagtacaactgaaggcagaaagtgttaggattttgcatctaatgttcattatcatggtattgatggacctaagaaaataaaaattagactaagcccccaaataagctgcatgcatttgtaacatgattagtagatttgaatatatagatgtagtattttgggtatctaggtgttttatcattatgtaaaggaattaaagtaaaggactttgtagttgtttttattaaatatgcatatagtagagtgcaaaaatatagcaaaaataaaaactaaaggtagaaaagcattttagatatgccttaatttagaaactgtgccaggtggccctcggaatagatgccaggcagagaccagtgcctgggtggtgcctcctcttgtctgccctcatgaagaagcttccctcacgtgatgtagtgccctcgtaggtgtcatgtggagtagtgggaacaggcagtactgttgagaggagagcagtgtgagagtttttctgtagaagcagaactgtcagcttgtgccttgaggcttccagaacgtgtcagatggagaagtccaagtttccatgcttcaggcaacttagctgtgtacagaagcaatccagtgtggtaataaaaagcaaggattgcctgtataatttattataaaataaaagggattttaacaaccaacaattcccaacacctcaaaagcttgttgcattttttggtatttgaggtttttatctgaaggttaaagggcaagtgtttggtatagaagagcagtatgtgttaagaaaagaaaaatattggttcacgtagagtgcaaattagaactagaaagttttatacgattatcattttgagatgtgttaaagtaggttttcactgtaaaatgtattagtgtttctgcattgccatagggcctggttaaaactttctcttaggtttcaggaagactgtcacatacagtaagcttttttccttctgacttataatagaaaatgttttgaaagtaaaaaaaaaaaatctaatttggaaatttgacttgttagtttctgtgtttgaaatcatggttctagaaatgtagaaattgtgtatatcagatactcatctaggctgtgtgaaccagcccaagatgaccaacatccccacacctctacatctctgtcccctgtatctcttcctttctaccactaaagtgttccctgctaccatcctggcttgtccacatggtgctctccatcttcctccacatcatggaccacaggtgtgcctgtctaggcctggccaccactcccaacttgacctagccacattcatctagagatggttcctgatgctgggcacagactgtgctcatggcacccattagaaatgcctctagcatctttgtatgcatcttgatttttaaaccaagtcattgtacagagcattcagttttggctgtggtaccaagagaaaaactaatcaagaatataaaccacattccaggctgctgttttctctccatctacaggccacacttttactgtatttcttcatacttgaaattcattctgctattttcatatcagggtacagacttataagggtgcatgttccttaaaggtgcataattattcttattccgtttgcttatattgctacagaatgctctgttttggtgctttgagttctgcagacccaagaagcagtgtggaaattcactgcctgggacacagtcttataagaatgttggcaggtgactttgtatcagatgttgcttctcttttctctgtacacagattgagagttaccacagtggcctgtcgggtccaccctgtgggtgcagcacagctctctgaaagcaagaaccttcctacctattctaacgtttttgccctctaagaaaaatggcctcaggtatggtatagacatagcaagaggggaagggctgtctcactctagcaaccatccctccattacacacagaaagccctcttgaagcaaaagaagaagaaagaaagaaagcttatctctaaggctactgtcttcagaatgctctgagctgaatgctcttgctcctttcccaagaggcagatgaaaatatagccagtttatctatacccttcctatctgaggaggagaatagaaaagtagggtaaatatgtaacgtaaaatatgtcattcaaggaccaccaaaactttaagtaccctatcattaaaaatctggttttaaaagtagctcaagtaagggatgctttgtgacccagggtttctgaagtcagatagccattcttacctgccccttactctgacttattgggaaagggagaactgcagtggtgtttctgttgcagtggcaaaggtaacatgtcagaaaattcagagggttgcataccaataatcctttggaaactggatgtcttactgggtgctagaatgaaaatgtaggtatttattgtcagatgatgaagttcattgtttttttcaaaattggtgttgaaatatcactgtccaatgtgttcacttatgtgaaagctaaattgaatgaggcaaaaagagcaaatagtttgtatatttgtaataccttttgtatttcttacaataaaaatattggtagcaaataaaaataataaaaacaataactttaaactgctttctggagatgaattactctcctggctattttcttttttactttaatgtaaaatgagtataactgtagtgagtaaaattcattaaattccaagttttagcagaaaaaaaaaaaaaaaaaa

SEQ ID NO: 6 - DNA, 11733 bp, Homo sapiens
cgccgctgtgcccaccccctcgccggagagagtgctggtaactccttccccagagtctgattacctgctccgcgaggccgcggacacgtgcggagagccgactgacactcgcagccccctcgggaggcccgacgcgactgggcccctcaggtgcaatgattctggactgagacgcgcttgggcagaggctatgtaatcgtgtctgtgttgaggacttcgcttcgaggagggaagaggagggatcggctcgctcctccggcggcggcggcggcggcgactctgcaggcggagtttcgcggcggcggcaccagggttacgccagccccgcggggaggtctctccatccagcttctgcagcggcgaaagccccagcgcccgagcgcctgagccggcggggagcaagtaaagctagaccgatctccggggagccccggagtaggcgagcggcggccgccagctagttgagcgcaccccccgcccgccccagcggcgccgcggcgggcggcgtccaggcggcatggagaaggacggcctgtgccgcgctgaccagcagtacgaatgcgtggcggagatcggggagggcgcctatgggaaggtgttcaaggcccgcgacttgaagaacggaggccgtttcgtggcgttgaagcgcgtgcgggtgcagaccggcgaggagggcatgccgctctccaccatccgcgaggtggcggtgctgaggcacctggagaccttcgagcaccccaacgtggtcaggttgtttgatgtgtgcacagtgtcacgaacagacagagaaaccaaactaactttagtgtttgaacatgtcgatcaagacttgaccacttacttggataaagttccagagcctggagtgcccactgaaaccataaaggatatgatgtttcagcttctccgaggtctggactttcttcattcacaccgagtagtgcatcgcgatctaaaaccacagaacattctggtgaccagcagcggacaaataaaactcgctgacttcggccttgcccgcatctatagtttccagatggctctaacctcagtggtcgtcacgctgtggtacagagcacccgaagtcttgctccagtccagctacgccacccccgtggatctctggagtgttggctgcatatttgcagaaatgtttcgtagaaagcctctttttcgtggaagttcagatgttgatcaactaggaaaaatcttggacgtgattggactcccaggagaagaagactggcctagagatgttgcccttcccaggcaggcttttcattcaaaatctgcccaaccaattgagaagtttgtaacagatatcgatgaactaggcaaagacctacttctgaagtgtttgacatttaacccagccaaaagaatatctgcctacagtgccctgtctcacccatacttccaggacctggaaaggtgcaaagaaaacctggattcccacctgccgcccagccagaacacctcggagctgaatacagcctgaggcctcagcagccgccttaagctgatcctgcggagaacacccttggtggcttatgggtccccctcagcaagccctacagagctgtggaggattgctatctggaggccttccagctgctgtcttctggacaggctctgcttctccaaggaaaccgcctagtttactgttttgaaatcaatgcaagagtgattgcagctttatgttcatttgtttgtttgtttgtctgtttgtttcaagaacctggaaaaattccagaagaagagaagctgctgaccaattgtgctgccatttgatttttctaaccttgaatgctgccagtgtggagtgggtaatccaggcacagctgagttatgatgtaatctctctgcagctgccgggcctgatttggtacttttgagtgtgtgtgtgcatgtgtgtgtgtgtgtgtgtgtgtgtgtgtgtgtatgtgagagattctgtgatcttttaaagtgttactttttgtaaacgacaagaataattcaattttaaagactcaaggtggtcagtaaataacaggcatttgttcactgaaggtgattcaccaaaatagtcttctcaaattagaaagttaaccccatgtcctcagcatttcttttctggccaaaagcagtaaatttgctagcagtaaaagatgaagttttatacacacagcaaaaaggagaaaaaattctagtatattttaagagatgtgcatgcattctatttagtcttcagaatgctgaatttacttgttgtaagtctattttaaccttctgtatgacatcatgctttatcatttcttttggaaaatagcctgtaagctttttattacttgctataggtttagggagtgtacctcagatagattttaaaaaaaagaatagaaagcctttatttcctggtttgaaattcctttcttccctttttttgttgttgttattgttgtttgttgttgttattttgtttttgtttttaggaatttgtcagaaactctttcctgttttggtttggagagtagttctctctaactagagacaggagtggccttgaaattttcctcatctattacactgtactttctgccacacactgccttgttggcaaagtatccatcttgtctatctcccggcacttctgaaatatattgctaccattgtataactaataacagattgcttaagctgttcccatgcaccacctgtttgcttgctttcaatgaacctttcataaattcgcagtctcagcttatggtttatggcctcgattctgcaaacctaacagggtcacatatgttctctaatgcagtccttctacctggtgtttacttttgttacctaaataatgagtaggatcttgttttgttttatcaccagcacacagattgctataaactgttactttgtgaattacatttttatagaagatattttcagtgtctttacctgagggtatgtctttagctatgttttagggccatacatttactctatcaaatgatcttttctccatcccccaggctgtgcttatttctagtgccttgtgctcactcctgctctctacagagccagcctggcctgggcattgtaaacagcttttcctttttctcttactgttttctctacagtcctttatatttcataccatctctgccttataagtggtttagtgctcagttggctctagtaaccagaggacacagaaagtatcttttggaaagtttagccacctgtgctttctgactcagagtgcatgcaacagttagatcatgcaacagttagattatgtttagggttaggattttcaaagaatggaggttgctgcactcagaaaataattcagatcatgtttatgcattattaagttgtactgaattctttgcagcttaatgtgatatatgactatcttgaacaagagaaaaaactaggagatgtttctcctgaagagcttttggggttgggaactattcttttttaattgctgtactacttaacattgttctaattcagtagcttgaggaacaggaacattgttttctagagcaagataataaaggagatgggccatacaaatgttttctactttcgttgtgacaacattgattaggtgttgtcagtactataaatgcttgagatataatgaatccacagcattcaaggtcaggtctactcaaagtctcacatggaaaagtgagttctgcctttcctttgatcgagggtcaaaatacaaagacatttttgctagggcctacaaattgaatttaaaaactcactgcactgattcatctgagctttttggttagtattcatggctagagtgaacatagctttagtttttgctgttgtaaaagtgttttcataagttcactcaagaaaaatgcagctgttctgaactggaatttttcagcattctttagaattttaaatgagtagagagctcaacttttattcctagcatctgcttttgactcatttctaggcagtgcttatgaagaaaaattaaagcacaaacattctggcattcaatcgttggcagattatcttctgatgacacagaatgaaagggcatctcagcctctctgaactttgtaaaaatctgtccccagttcttccatcggtgtagttgttgcatttgagtgaatactctcttgatttatgtattttatgtccagattcgccatttctgaaatccagatccaacacaagcagtcttgccgttagggcattttgaagcagatagtagagtaagaacttagtgactacagcttattcttctgtaacatatggtttcaaacatctttgccaaaagctaagcagtggtgaactgaaaagggcatattgccccaaggttacactgaagcagctcatagcaagttaaaatattgtgacagatttgaaatcatgtttgaatttcatagtaggaccagtacaagaatgtccctgctagtttctgtttgatgtttggttctggcggctcaggcattttgggaactgttgcacagggtggagtcaaaacaacctacatataaaaagagaaaaagagaaacttgtccatttagctttcataagaaatcccatggcaaagggtaataaaaaggacctaatcttaaaaatacaatttctaagcacttgtaagaacccagtgggttggagcctcccactttgtccctcctttgaagtggatgggaactcaaggtgcaaagaacctgttttggaagaaagcttggggccatttcagccccctgtattctcatgattttctctcaggaagcacacactgtgaatggcagacttttcatttagccccaggtgacttactaaaaatagttgaaaattattcacctaagaatagaatctcagcattgtgttaaataaaaatgaaagctttagaaggcatgagatgttcctatcttaaataaagcatgtttcttttctatagagaaatgtatagtttgactctccagaatgtactatccatcttgatgagaaaactcttaaatagtaccaaacattttgaactttaaattatgtatttaaagtgagtgtttaagaaactgtagctgcttcttttacaagtggtgcctattaaagtcagtaatggccattattgttccattgtggaaattaaattatgtaagcttcctaatatcataaacatattaaaattcttctaaaatattgcttttcttttaagtgacaatttgactattcttatgataagcacatgagagtgtcttacattttccaaaagcaggctttaattgcatagttgagtctaggaaaaaataatgttaaaagtgaatatgccaccataattacttaattatgttagtatagaaactacagaatatttaccctggaaagaaaatattggaatgttattataaactcttagatatttatataattcaaaagaatgcatgtttcacattgtgacagataaagatgtatgatttctaaggctttaaaaattattcataaaacagtgggcaatagataaaggaaattctggagaaaatgaaggtatttaaagggtagtttcaaagctatatatattttgaaggatatattctttatgaacaaatatattgtaaaaatttatactaaggtcatctggtaactgtgggattaatatggtcgaaaacaaatgttatggagaagctgtcccaagcaaactaaattacctgtacttttttcccatttcaagggaagaggcaaccacatgaagcaatacttcttacacatgcctaagaacgttcattgaaaaaataaatttttaaaaggcatgtgtttcctatgccaccaatacttttgaaaaattgtgaaccttacccaaaaccatttatcatgtccattaagtatatttgggtatataattaggaagatatttacatgttccatctccacagtggaaaaacttattgaggctaccaaagtgtgccaagaaatgtaagtccttagagtaattagaaatgctgttttcctcaaaagcatgagaaactagcattttcatttcttatttactccctttctatatcaatgcaattcacaacccaattttaatacatccctatatctcaagcatttctatcttgtactttttcagaaaataaaccaaaaataatcctttggtctctctatcttctgacctttgtaagcaacagaaatgtaaaaacagaaggggtccaatttttacacgtttttttctcaagtagcctttctggggatttttattttcttaatgaagtgccaatcagcttttcaaaatgttttctatttctcagcatttccaggaagtgataacgtttagctaaatgagtagaagtggacttccttcaacatattgttaccttgtctagccttaggaagaaaacaagagccacctgaaaataaatacaggctcttttcgagcatctgctgaaatactgttacagcaatttgaagttgatgtggtaggaaaggaaggtgacttttcttgcaaaagtctttctaaacattcacactgtcctaagagatgagctttcttgttttattccggtatattccacaaggtggcacttttagagaaaaacaaatctgatgaagactaaagaggtacttctaaaagagatttcattctaactttatttttctgcgcatatttaactctttcctagcacttgttttttgggatgattaatagtctctataatgttctgtaacttcaatattttacttgttacctaggttctgaacaattgtctgcaaataaattgttcttaaggatggataatacacccattttgatcatttaagtaaagaaagcctagtcattcattcagtcaagaaaaaatttttgaagtacccagttaccttacttttctagattaaaacaggcttagttactaaaaaggcagtcctcatctgtgaacaggatagtttcgttagaagtataaaactcctttagtggccccagttaaaacacacataccctctctgctgctttcaaattccctagcatggtggcctttcaacattgattaaattttaaaatcctaatttaaagatcaggtgagcaaaatgagtagcacatcagtaattcagtagacaaaacttttgtctgaaaaattgctgtattgaaacagagccctaaaataccaaaagaccaggtaattttaacatttgtggaatcacaaatgtaaattcataagaagctctaattaaaaaaaaaaagtctgaagtatatgagcataacaacttaggagtgtgtctacatacttaacttttgaagttttttggcaactttatatactttttttaaatttacaagtctacttaaagacttcttataccccaaatgattaagttaattttagaggtcacctttctcacagcagtgtcacttgaaatttagtagggaaggatattgcagtatttttcagtttccttagcacagcaccacagaaagcagcttattccttttgagtggcagacactcgacggtgcctgcccaactttcctcctgagtggcaagcagatgagtctcagtaattcatactgaaccaaaatgccacatacactaggggcagtcagaaactggctgagaaatcccccgcctcattcgcccctctgctcccaggaactagagtccagttaaagcccctatgcgaaaggccgaattccaccccagggtttgttataacagtggccagtctgaaccccatttgctcgtgctcaaaacttgattcccacttgaaagccttccgggcgcgctgcctcgttgccccgcccctttggcaggagagaggcagtgggcgaggccgggctggggccccgcctcccactcacctgccggtgcctgaaattatgtgcggccccgcgggctgctttccgaggtcagagtgccctgctgctgtctcagaggcatctgttctgcaaatcttaggaagaaaaatgtccctagtagcaaacgggtgtcttctgtgcataaataagtacaacacaattctccgaaagttcgggtaaaaagagatgcggtagcagctgccctgtgtgaagctgtctaccccgcatctctcaggcgctaagctcagtttttgtttttgtttttgtttttttaaagaaaagatgtataattgcaggaatttttttttatttttttattttccatcattctatatatgtgatggtgaaagatatgcctggaaaagttttgttttgaaaagtttattttctgcttcgtcttcagttggcaaaagctctcaattctttagcttccagtttcttttctctctttttctttgttaggtaattaaaggtatgtaaacaaattatctcatgtagcaggggattttcatgttgagaggaatcttccgtgtgagttgtttggtcacacaaataaccctttctcaattttaggagtttggattgtcaaatgtaggtttttctcaaagggggcatataactacatattgactgccaagaactatgactgtagcactaatcagcacacatagagccacacaattatttaatttctaactctctgtggtccctagaaaaattccgttgatgtgcttaggttaaagttctgaagatacccgttgtacccttacttgaaagtttctaatcttaagttttatgaaatgcaataatatgtatcagctagcaatatttctgtgatcaccaacaactctcagtttgatcttaaagtctgaataataaaacaaatcccagcagtaatacatttcttaaacctcacagtgcatgatatatcttttcattctgatcctgtgtttgcaaaaatatacacatgtatatcatagttcctcactttttattcatttgttttcctattacctgtagtaaatatattagttagtacatggaatttatagcatcagctacccccaggaacagcacctgacaggcgggggattttttttcaagttgttctacatttgcataaattatttctattattattcatgtatgttatttatttctgaatcacactagtcctgtgaaagtacaactgaaggcagaaagtgttaggattttgcatctaatgttcattatcatggtattgatggacctaagaaaataaaaattagactaagcccccaaataagctgcatgcatttgtaacatgattagtagatttgaatatatagatgtagtattttgggtatctaggtgttttatcattatgtaaaggaattaaagtaaaggactttgtagttgtttttattaaatatgcatatagtagagtgcaaaaatatagcaaaaataaaaactaaaggtagaaaagcattttagatatgccttaatttagaaactgtgccaggtggccctcggaatagatgccaggcagagaccagtgcctgggtggtgcctcctcttgtctgccctcatgaagaagcttccctcacgtgatgtagtgccctcgtaggtgtcatgtggagtagtgggaacaggcagtactgttgagaggagagcagtgtgagagtttttctgtagaagcagaactgtcagcttgtgccttgaggcttccagaacgtgtcagatggagaagtccaagtttccatgcttcaggcaacttagctgtgtacagaagcaatccagtgtggtaataaaaagcaaggattgcctgtataatttattataaaataaaagggattttaacaaccaacaattcccaacacctcaaaagcttgttgcattttttggtatttgaggtttttatctgaaggttaaagggcaagtgtttggtatagaagagcagtatgtgttaagaaaagaaaaatattggttcacgtagagtgcaaattagaactagaaagttttatacgattatcattttgagatgtgttaaagtaggttttcactgtaaaatgtattagtgtttctgcattgccatagggcctggttaaaactttctcttaggtttcaggaagactgtcacatacagtaagcttttttccttctgacttataatagaaaatgttttgaaagtaaaaaaaaaaaatctaatttggaaatttgacttgttagtttctgtgtttgaaatcatggttctagaaatgtagaaattgtgtatatcagatactcatctaggctgtgtgaaccagcccaagatgaccaacatccccacacctctacatctctgtcccctgtatctcttcctttctaccactaaagtgttccctgctaccatcctggcttgtccacatggtgctctccatcttcctccacatcatggaccacaggtgtgcctgtctaggcctggccaccactcccaacttgacctagccacattcatctagagatggttcctgatgctgggcacagactgtgctcatggcacccattagaaatgcctctagcatctttgtatgcatcttgatttttaaaccaagtcattgtacagagcattcagttttggctgtggtaccaagagaaaaactaatcaagaatataaaccacattccaggctgctgttttctctccatctacaggccacacttttactgtatttcttcatacttgaaattcattctgctattttcatatcagggtacagacttataagggtgcatgttccttaaaggtgcataattattcttattccgtttgcttatattgctacagaatgctctgttttggtgctttgagttctgcagacccaagaagcagtgtggaaattcactgcctgggacacagtcttataagaatgttggcaggtgactttgtatcagatgttgcttctcttttctctgtacacagattgagagttaccacagtggcctgtcgggtccaccctgtgggtgcagcacagctctctgaaagcaagaaccttcctacctattctaacgtttttgccctctaagaaaaatggcctcaggtatggtatagacatagcaagaggggaagggctgtctcactctagcaaccatccctccattacacacagaaagccctcttgaagcaaaagaagaagaaagaaagaaagcttatctctaaggctactgtcttcagaatgctctgagctgaatgctcttgctcctttcccaagaggcagatgaaaatatagccagtttatctatacccttcctatctgaggaggagaatagaaaagtagggtaaatatgtaacgtaaaatatgtcattcaaggaccaccaaaactttaagtaccctatcattaaaaatctggttttaaaagtagctcaagtaagggatgctttgtgacccagggtttctgaagtcagatagccattcttacctgccccttactctgacttattgggaaagggagaactgcagtggtgtttctgttgcagtggcaaaggtaacatgtcagaaaattcagagggttgcataccaataatcctttggaaactggatgtcttactgggtgctagaatgaaaatgtaggtatttattgtcagatgatgaagttcattgtttttttcaaaattggtgttgaaatatcactgtccaatgtgttcacttatgtgaaagctaaattgaatgaggcaaaaagagcaaatagtttgtatatttgtaataccttttgtatttcttacaataaaaatattggtagcaaataaaaataataaaaacaataactttaaactgctttctggagatgaattactctcctggctattttcttttttactttaatgtaaaatgagtataactgtagtgagtaaaattcattaaattccaagttttagcagaaaaaaaaaaaaaaaaaa

SEQ ID NO: 7 - protein, 326 aa, Homo sapiens
MEKDGLCRADQQYECVAEIGEGAYGKVFKARDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDVIGLPGEEDWPRDVALPRQAFHSKSAQPIEKFVTDIDELGKDLLLKCLTFNPAKRISAYSALSHPYFQDLERCKENLDSHLPPSQNTSELNTA

SEQ ID NO: 8 - DNA, 11534 bp, Mus musculus
cccgcgctgcgctcatccccgaggggccccagcaacctctccttcgtgaagactgcacgagccctgctgtggaagaaaagtgcagagattgtgggcagactatgtaatcgctgcggagggggaagaggagggatcggcgctctcctgcggcggcggcgcggcgactcggctaggcggagtttcgcagcggctgcgcccggcttgcacccgcgggcgagaaggtcggtccgtctagcccggcggccgcgagtccgactccccgaggcgtgtaaggcagcgagtgagcaccccggttccactgtgccgcacccgcagcctgaagccagcatggagaaggacagcctgagtcgcgccgatcagcagtatgagtgcgtggcggagatcggcgaaggcgcctatgggaaggtgttcaaggcccgcgacctgaagaacggcggccgcttcgtggctctgaagcgcgtgcgagtgcagaccagtgaggagggcatgccgctctccaccatccgcgaggtggcggtgctgaggcacctggagaccttcgagcaccccaacgtggtcaggttgtttgatgtgtgcacagtgtcacggacggacagagaaaccaagcttacactagtgtttgagcatgttgatcaagacttgaccacttacttggataaagttccagagcccggcgtacccacagaaaccataaaggatatgatgtttcagcttctccgaggtctggactttcttcattctcacagagtagtgcatcgtgatctgaaaccgcagaacattctggtgaccagcagtggacagataaagctggctgactttggccttgcccgcatctatagttttcagatggcccttacctcggtggtcgtcacgctgtggtaccgagccccagaagtcctgctccagtccagctatgccacccctgtggacctctggagtgtcggttgcatctttgcagaaatgtttcgcagaaagcctctttttcgtggaagttcagacgtggatcaactaggaaaaatcttggacatcattggactcccaggagaggaagactggcctagggacgtggcccttccccggcaggcttttcattccaaatctgctcaacccatcgagaagtttgtgacagatattgacgaactaggcaaagacctacttctgaaatgcctgacgtttaatccagctaaaaggatatccgcctacggcgccctgaatcacccgtacttccaagatctggagagatacaaggacaacctgaactctcacctgccatccaaccagagcacctcggagctgaacacagcctgaggttccacggggatgcccatgagctcgtcatctgaacacattggcggctgcgagtcccctaagcaagcctctcagagcagttgaagattgctggctgccaaccttctggctgccagcttctgggtgggctctgccttaccaaggaaaccacctagtttactgttcagagatcaatgcaagggtgattgcagctttatgttcgtttgtacacttgtttgttttgtctgtttgtttcaagaacctggaaaacttccagaagaagagaagctgctgaccaattgtgctgccatttcgttttctaaccttgaatgctgccagtgtagggtgggaatccaggcccagctgagttatgatgtaatccgcctgcagctgctgggcctgctttggtacttgtgagtgtgtgtgcatgcgtatgtgtgtgtaagagagaagaggaggggagagaaagacccctgatctcgtcaagtgttactttttttttgtagaaaacaagaataattgagttttaaagagtagaggtgactgatagtaagaagggcttgttcagtgaaaggtgattcacaatggagtcttgttaggaaggttggacctaagtcctcagagttgccttcctgtccaaaagcttttgctagcagtaaacaataaaggtttagatgccacaaaaaatggggggaaccacaatattttttaagagactttttaaggcatacatcttctatttactctttggaaagctgaacttaatgtgtcccaggccctatatatagtacagtatgtacttaattgtttctttggggaaagatgctataagtatcttattacttgcaatacatttaaggagtgagtgtacctcagataggttttaaagatagagagcacctgttttctggtgtgagatgttatcattttcttcacgtctcttgataccttgataccttgtcaccttagggaatcacttcctgctctgactagaggcgggaataccatctagctgtctccaccacccaccatggcgcatctgccttgtgctgccttgtgtagtgcgaagctctcaaccaccagcacttctaattcattttcctgccactgcctggctaacgacagatggcccagctgccccaatcccacacccgcttgcacgcttaccgtctttcaccgaatgctttgggcgtaggctcccattccgaaaccctaacagtatccccttgtgcctttgtaatacagtcttccccctgccgcagctgaggtcacctaggcagtgaagagtgcttgttctgtgtgtgtatagactactaccgactgtcacttggtgtttcctatctttaagtgtatgttgtcagtgtaatgtctgaggaaatgtcttttcctctcttctagagataactacttactctctaaagtgatctctctgtctgtccgcaggatgtgtttctgggtttttgttctttttttttttttttttttttttttgtctgaggcctcatgtcagccctgctttctgcagagccagcctaccctgggcattataaacaactgtaccttactgttttctttcagtcctttacattctgtgccacctttgccttattatatccgtggcttagtgctaagctggcactactccatagaaggtgtagaaggcagctttcgggaagcgtagctgagttttgtttgaatcgtgtttgagcacatttaagtaaacgctagatgatgcttcactgtagagctttgaaagactgcctgatgtttcacacggcagtgtaggctccagatggtgtttgtatgttatggaccttactgtgtttctcgtagctgaacaatgcatgtggttatttgtttgtttgttttttggtttctctgtgtagccctggctgtcctggaactcactctgtagaccaggctggcctcgaattcagaaatgcgcctgcctctgcctcccaagtgctgggatcaaaggcgtgcgccaccacgcccggctttgcatatggttatctgtatggagaagaattagatgtctttcctgagagctttggaggttgggaatcatcttcttctgtttatttttatttttaacattacattacattcttctgattcagtatcgcaagaaaggattttttcttttttcaaaaacaaaactttaagaagccagaggctaggctgagaatgttctgcgctcgtcctcacagctctgctgccgacactgccaacgctcgagacagtcagcctggaacctctgggacaaggtctactcgaggtctcacttggacagcgagttctgccttgtcattgaacaaggtccaaaatacagacatttttgctagggcctagaaatcgaccataaaactcactgcattgaggcctaatccattctatattaacatccatgtgataaagtgaaaataattttgagggttttgctttcgccaaaaaaaaaaaagaaagaaagaaaaaaaaagaaaggaagaaaaagaaaaaagaaaggattgttttccttagctttgctcaagaaaaatacagctatcctaaactggaatcttcccgtattctttagagtcttaagtatagagtttaacttcacttttgtcagctgtcttgagcttatttctaagtagttttgttttggcttttggtgagtgaggtagtgggaggttaattttggggtcttttttttttttggctttgtttgtgtgtgtgtgtgtgtgtgtgtgtttatgtgtttggcttttgggtttggttggttagttgtttagtttgtttttttgttgttgtttaatttgctttttctaggcagggtttctctatgtggccctggctgtcctagaacttgctctgtagaccaggctagcctcaaactcagagatccacttgcctctgtctcgagtgtgcttgaactaaaggcatgagctaccctcctcagcccactccaaactttgagtaatatttttaaatcatagacatgttggtattagaaacatggcgtattgtggtggatggtagagaaagggcgccgcaacctctcaggacatcatatgtctggccagctcttcattcatataggtgctctctggatatgtctttacatcagactcacacctctgaaacccggttttcacacaaggagacttgccattggggtctttagtagcccagggaaacatagagtaaactgaagagcttactgtgtgccgcttatccttagatggcctcatccagtcacaggtctttgtcagaatctaaccattgattcactgaggagaccctactgccctgaggttccactgaagcagcttttagtgagactagggcaagaatataccgaatagcttctgttcaaggtttggttccagagactctaccatttacaggactggctggagttacaacaacctcttaaagagggtagggcgggttctccagttagaccccctaacctcacaggtgagtagtcctaacctgacagccttagaaacacagttgccgagtacctgagagcacccagtggcccgccgcctcctattcagcccctctttaaagacgacagacggttaagccatggagaagctgttcaggtagcacggggccatttctgcccccagcttgtcatgttttttctctcaggacccactctctgaccagtagacttgaatttagccccaagtgatttactaaactcagctcagaagctcacttaaaaataggacctccacatggtcgtgctaagtaaaaatgaagaccccaagttgcacagggtgctgctatctcaaagacaatactttcctttgaatagaaagctgtgccgtccaccctgcaatgggtgctagccaacttgcttgagaaagtcaggagcaagcattttgagttttaaagaatgtcctgaacttgtcttttaagtaaacatagctgctttattaaaagctgtgactactaaagccagtcacagccatcgtcactgtgggagttaaatcatgtcaactttcaaacatcacagttgtttcaatcttctgtccaaagaactcctcttctggagaaaacctgattttggttttggctttttgtcttgtcttgttttgtttttcctgagatggggtctctctgtgaagccctggctatcctggaactcagtgtgtagaccaggctggccttgaactcatgtagatctgcctgctaaaactaaagacatgcaccaccatgacagctgactattttttatgatgagcacagaagtggctcacattttcttaaagcacacttttgtcacatagtagagtctaggaaagaataacattgaaaatccccatcccagtgtgacacttgtgtaagcacatatggatagccattggaagaatacatgcttgcttgtaacatgaaattaaagacattgtataatttcttaatctttaaaaattaccaaaaaagtggcaacagtatgtttaaggttattttaatttttacataaattgttaaccatatttgtttgcctttaagtatattctaagaattataattaagtataattataagtcatcccgcacattcagcacttatggactaggtatgtcaaaatacacaaattctgtatttcttgtttcataggaagaaagcagccatctgaagctctactcatatatatgcctaagaatatctatacaaaattaaagtttaaaaggcctttgcctctcgtgcagctgagaatagacaacttataagccttatccaaaaaccatgataaagggatcatgtgcatccttacaaaaaccccttatcctgtgcatacttacatccaagtacatcctaaggaaggtgtccgtgcattcatctgccacagtggggaagctgcctggtgcaaggtgtgagtcctggaatgattagtgatagtggtctcaaatgaggacaccttcgtctcccaccagtttccttcctatggacaggctcagtccagttatacaggccaggaggggctggtgcccacagcctcccgcctctgcaaggtaggattgcatatctcaaacatctctaactcagtttcggctttgttggtcttgtttaaggaaataagccagtaagccaaaactatttttgttgttgttctcttgtcttctgaaagagaggggatatcaattttaaatgtttctctcaatttgatctttcaaatttttctaataaagtgccaaattgctgctgcttcttcttcttctttcttcttcttcttcttcttcttcttcttcttcttcttcttcttcttcttcttcttcttcttcttcttcttcatgttttctgctagggttttcttgttttgttttggtttcagtttgttttattttcagcaagttaggtaacttgagaggacttccactaggatgttcacacctctgtgctctggtcctctgacccctccaaaaaatagccacctgggaataaatgcaagctccccccctccagcatctaccaaaatactgttgcagcaattccaagttcaaatggtggggatggaaggtagtttggagttgggttgggttgggttgggttaggttggacagaagtcagttttaaaattcacagcgccctaaggtcagtgtggggtttattcctctatcccaatgtgtttcacaaggtggcgctttcagagaaaactctaaacctgaccaagactgagaggtattctagagacagtacttgtaactattttcctgcacatctgtaactatcacctagcatattccttcagacaattaacagccttcataatattctacagcttatatgttttgctttttagctaggttctgagtgactaagttaggcttgagagtgatgtttaagcaataaagaaggcctttacttaagagacatttttgatgtacccaaataacctgaaattctaggacatacaactaaaaacttccctgaagccggccctcgtctgtgagcagcacggtttcattagaaatggcaaaactccttgcggggttgggagtgggggacagtcctaactctcacagcccttgacatgtctgctgcggttcacccctcactatccatgaccttttgacactgtcatttctaaactcaagtgaacaaaggaagcactgcatctgtaactgagtatgcagaccattttctaaagaaccacagtgctcgacaaatactggaagaactggtatgttttacacgcacataaactacaagtgcaaactcttacattccaattcctaaattacagttatatagtacatatgaacattacagtttagacaggaacctgcataaggctttggtttttctactctcgaggtcacttttatactacattttaaattcacacttttctcagagacccagccccctgagccaatagtgacagtacactgggatgaacagacacagtaccacaccatggcagaaagcagtttattccttatgagtcacacacacacacacacacaccctgcctgacttcacacacacacacacacactgcctgacttcacacacacacaccctgcctgacttcactccagaaaggctggcagggaggtttcagtcatttacacagaccaggtgctcaagcattgagaaatcgattctttcaaccctctgctctcactgaagcagatctcaggttaagcccctcagtgaaaggccaagggtcccctgggtctgccaccattctggagttcctgagcctgaccccacccctactcctgctgaatgacagggggcggggcagcggagccaggccaagcaggatattgtgggaggaccagtgacctgctgcacctcagccatctgttgtgccatcttaggaagcagggtgctcgagaaacacagagctgtcctctgagctacaataactacagtttgctcactaggctagctagctctggtgtagagatgcaagccctatatgaaatgtctcccactcaaagttttgctggggggtgggggttgagatgtataattgcaggagaaattctgactttactgttttctatctgatacatccatggtggggaaaagatatgaaactgtctcagtttgaaaagctcttctacttcaattggcaaacgcatggagttcgttactttctactttcttcttttttctcctttagtgagcaaattattccttgaacagaaagaggattttcccatttagaggaagcttctgtgtgaattgtcaggtcacacaaataatgccattatattgttgagtgtagggctttggtttttcaggggacacatagccaagcagtgatggtcaggaattatgtctcactctatcatttcttactcaagcagggttccagccagattctattgtgagctgaggttagtcgggggaccctgtgctctacttgaagtttcataatctttagttctatgaaatgcaataacatatattagccagcaatattcctgtggtcgccagcatctctgagtttgaccttcagtctctggatggttaaaatagatcccagcagtaatccatttcttgacatcagtgcttaagtcaagtatctttccactctggtcctatttgcaacaatgtattcatgtatgtttcatttcctttattcattattttcctatgaacagtgataaatgtattagcacattgaattaatagcataatagaaagagcacttagcagatggggattttttatcaagtcccacatttacataagttatttctattattattcatgtatattatttattgctaagcctcacaccattcctgtgaaagtgcaactaaaggcagatattagggcttggatttaatgttccttatcatggtcttgaacaaaaaattcagtgatgcccccaaataagctgcctgcgtttttaacacgactgctagatttcagtaggttaagtgtaacacttttggtgtctaggtgtctttttacattaaaagaggcaaagcagcagaactctgtagctattgcttctggcagagatttgtttagcatagtgtggtattaattatagcaaatgttaaggtagcatgtagaggttgccttcaataagggactgtgccaggtggccagcaaagtggattctaggcagagcaggcgtctaggtgctgcctcctgtctggttgtgtgaagagagctgctccgagcaatgtagtgcccccgtgcccccgtaggtgtcctgggcagcggtgggaacacgtggtactgtagagaagggatcagcgtgaggggtgttctgtaggaacagaactggtagcgtatgccttgaggcttcctgcccgtgttttcagattccaggcatacattctgtatacagaagcagtccatcctagcaataaaacctaggaatccccaacaactcaaaaggcagttgccctgctgtggtttctgtcctgagctgaaggttaacgggcaagtgttaggcacagaagagcatcgtgtgctggggtgccgtgccacggcccagggtgcgtgttagcagtagagactgttacagggttgtcccttctaggtgcgtagaagtatatggcttcactgtaaagtgtgtattgtttctgtattcccacagggacttgttaaagcttctagaggcttcagcaaagctgtagaccacagtaagctgctttttgtttgacttattcttagactaggaaatgtttaaaggttaaaaaaaaaaaaaaaagaatcttcatttagacactgaactcgctattttctggactgaaatcatggtcctagaactgtagaaatcacacccattagatgctaatctaggttgtgtaagccagccccaagagtctagcatccccacactgtatctgcatcttcctgcatcttaccttccttcccgaaggtgctccctaccactaccctgacctgtccctttggtgcttcccatctccttggctgaaagttaggcatcaagcatgtgatgagaaatactttcagcaccaagtatgcctgcctaggcctagccaccgcccccactttgcctttggctagccatagtcaacaagagataatacctggtgctgggcacagaatgaacccatgatgtcagtcagaaaagccctcaagcatatttctcctgtacgtcctgatctttaaacccagtcattatgcagagcgttcgctgttggctatagtcccaggttaaaaaaaaaaaaaacattccaggctgctgtaggctgtccatatgcagtctctgcttttaccttgctttttcatacttgaaattcgttctgccattttgatttcacggtgcagacatcagggtacacgttccctctgggtgcataattgctctcattccgtttgctcacactgctacaggatgctctgttttggtgctttgggttcagcgggtcaaaaagcactgtgggaaatcactgcctagaacacagtcttaaaagcatttgggcaggagtcttggtgttgatgtcgcttctgctcttctctgtgccctccagatggagttaccaggttggcctgccaggtcccccactcccacccccaagacctttctctgaatggcctgtgaaccctgtgctgtatccaagggtgaatgtgacgtatctcctctggcgaccctacctttcctggttactcaccatcagaaacaacctaggttacatatagcaaactctacacagccttcctgagggaaagcataaaaggatatctctaaggttgctcttcaaagccctgagctgaaggcctttgctcccgtccttggagctgatgaaaatactggtcaatttatccaagcccttcccacttacaagaatgggaaagtagaggtttgtgtcgataccatctaaagaccacaacttctagccatagggtatttcatatatgtccattttcaaaagcagctcaggtgagtggtgactccaggctccatgcaggcatcttaacatgggacttccctagagagagctacagtgttcattctatgtcacagggcaggagaggagaccggcaggaaatttggagggtaacagatcaatactcctttggaaactggatgtcttactgggtgctagaatgcaaatttatgtatttattgtctggtcattgaattcattgcctttcaaaaccattgttcaaatgtcactatcattggcctcacttgtatgccagccgaattggttgaaagcaaaccagagaatggtttgtgcatttgccttaccatttgtacttcttacaataaaaatgtcagtagcaaataaaaaaaaaaaaaa

SEQ ID NO: 9 - protein, 326 aa, Mus musculus
MEKDSLSRADQQYECVAEIGEGAYGKVFKARDLKNGGRFVALKRVRVQTSEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDIIGLPGEEDWPRDVALPRQAFHSKSAQPIEKFVTDIDELGKDLLLKCLTFNPAKRISAYGALNHPYFQDLERYKDNLNSHLPSNQSTSELNTA

SEQ ID NO: 10 - DNA, 21 bp, Artificial Sequence
  /note=“Description of Artificial Sequence Synthetic primer sequence”
atgaacgctacacactgcatc

SEQ ID NO: 11 - DNA, 21 bp, Artificial Sequence
  /note=“Description of Artificial Sequence Synthetic primer sequence”
ccatccttttgccagttcctc

SEQ ID NO: 12 - DNA, 21 bp, Artificial Sequence
  /note=“Description of Artificial Sequence Synthetic primer sequence”
ggacttgccttgttccgagag

SEQ ID NO: 13 - DNA, 22 bp, Artificial Sequence
  /note=“Description of Artificial Sequence Synthetic primer sequence”
gctgccacataactgatagcga

SEQ ID NO: 14 - DNA, 21 bp, Artificial Sequence
  /note=“Description of Artificial Sequence Synthetic primer sequence”
ctggcggacatggctttactt

SEQ ID NO: 15 - DNA, 21 bp, Artificial Sequence
  /note=“Description of Artificial Sequence Synthetic primer sequence”
ctcccacttttagcagtcccc

SEQ ID NO: 16 - DNA, 21 bp, Artificial Sequence
  /note=“Description of Artificial Sequence Synthetic primer sequence”
ggcctgtctaagaaacctgcc

SEQ ID NO: 17 - DNA, 23 bp, Artificial Sequence
  /note=“Description of Artificial Sequence Synthetic primer sequence”
ccaccttgaagtatagctttggg

SEQ ID NO: 18 - DNA, 23 bp, Artificial Sequence
  /note=“Description of Artificial Sequence Synthetic primer sequence”
taatggagactcattcccttgga

SEQ ID NO: 19 - DNA, 22 bp, Artificial Sequence
  /note=“Description of Artificial Sequence Synthetic primer sequence”
aaagtcagagtgctgaggtttg

SEQ ID NO: 20 - DNA, 21 bp, Artificial Sequence
  /note=“Description of Artificial Sequence Synthetic primer sequence”
gctgagagcatccgactgaac

SEQ ID NO: 21 - DNA, 21 bp, Artificial Sequence
  /note=“Description of Artificial Sequence Synthetic primer sequence”
aggtacatcaagctcgaagca

SEQ ID NO: 22 - DNA, 23 bp, Artificial Sequence
  /note=“Description of Artificial Sequence Synthetic primer sequence”
tagtccttcctaccccaatttcc

SEQ ID NO: 23 - DNA, 21 bp, Artificial Sequence
  /note=“Description of Artificial Sequence Synthetic primer sequence”
ttggtccttagccactccttc

SEQ ID NO: 24 - DNA, 21 bp, Artificial Sequence
  /note=“Description of Artificial Sequence Synthetic primer sequence”
gaggctatccagcgtactcca

SEQ ID NO: 25 - DNA, 21 bp, Artificial Sequence
  /note=“Description of Artificial Sequence Synthetic primer sequence”
cggcaggcatactcatctttt

SEQ ID NO: 26 - DNA, 20 bp, Artificial Sequence
  /note=“Description of Artificial Sequence Synthetic primer sequence”
accctcgtcctgctactctc

SEQ ID NO: 27 - DNA, 21 bp, Artificial Sequence
  /note=“Description of Artificial Sequence Synthetic primer sequence”
ctgtctcctcgtcccaatact

SEQ ID NO: 28 - DNA, 20 bp, Artificial Sequence
  /note=“Description of Artificial Sequence Synthetic primer sequence”
cagttcgtgaggttcgacag

SEQ ID NO: 29 - DNA, 19 bp, Artificial Sequence
  /note=“Description of Artificial Sequence Synthetic primer sequence”
cagccgtacatgctctgga

SEQ ID NO: 30 - DNA, 21 bp, Artificial Sequence
  /note=“Description of Artificial Sequence Synthetic primer sequence”
ggacaagagcagagatacacg

SEQ ID NO: 31 - DNA, 20 bp, Artificial Sequence
  /note=“Description of Artificial Sequence Synthetic primer sequence”
caaggacagctaggacaacc

SEQ ID NO: 32 - DNA, 23 bp, Artificial Sequence
  /note=“Description of Artificial Sequence Synthetic primer sequence”
cagcttgactcaaaattcctgga

SEQ ID NO: 33 - DNA, 23 bp, Artificial Sequence
  /note=“Description of Artificial Sequence Synthetic primer sequence”
tgaagattacgcttgcttttcct

SEQ ID NO: 34 - DNA, 23 bp, Artificial Sequence
  /note=“Description of Artificial Sequence Synthetic primer sequence”
actcacctcttcagaacgaattg

SEQ ID NO: 35 - DNA, 23 bp, Artificial Sequence
  /note=“Description of Artificial Sequence Synthetic primer sequence”
ccatctttggaaggttcaggttg